(19) United States
(12) Patent Application Publication
(10) Pub. No.: US 2022/0378363 A1
(43) Pub. Date: Dec. 1, 2022

(54) SYSTEMS AND DEVICES FOR DETECTING BIOMARKERS IN SITU AND RELATED METHODS

(71) Applicants: Massachusetts Institute of Technology, Cambridge, MA (US); Trustees of Boston University, Boston, MA (US); The Brigham and Women's Hospital, Inc., Boston, MA (US)

(72) Inventors: Timothy Kuan-Ta Lu, Cambridge, MA (US); Rabia Tugce Yazicigil Kirby, Newton, MA (US); Carlo Giovanni Traverso, Newton, MA (US); Jenna Ahn, Stanford, CA (US); Maria Eugenia Inda, Cambridge, MA (US); Miguel Jimenez, Malden, MA (US); Qijun Liu, Allston, MA (US); Phillip Nadeau, Cambridge, MA (US); Christoph Winfried Johannes Steiger, Berlin (DE); Adam Wentworth, East Boston, MA (US)

(73) Assignees: Massachusetts Institute of Technology, Cambridge, MA (US); Trustees of Boston University, Boston, MA (US); The Brigham and Women's Hospital, Inc., Boston, MA (US)

(21) Appl. No.: 17/730,075

(22) Filed: Apr. 26, 2022

Related U.S. Application Data
(60) Provisional application No. 63/310,052, filed on Feb. 14, 2022.
(60) Provisional application No. 63/255,144, filed on Oct. 13, 2021.
(60) Provisional application No. 63/179,804, filed on Apr. 26, 2021.

(51) Int. Cl. A61B 5/00; C12Q 1/6897; A61B 5/145; A61B 5/1459

(52) U.S. Cl. CPC A61B 5/4255; C12Q 1/6897; A61B 5/6861; A61B 5/14507; A61B 5/14546; A61B 5/1459; A61B 2562/028; A61B 2560/04

(57) ABSTRACT

Transient molecules in the gastrointestinal (GI) tract, such as nitric oxide and hydrogen sulfide, are important signals and mediators of inflammatory bowel disease (IBD). Because these molecules may be short-lived in the body, they are difficult to detect. To track these reactive molecules in the GI tract, a miniaturized device has been developed that integrates genetically engineered probiotic biosensors with a custom-designed photodetector and readout chip. Leveraging the molecular specificity of living sensors, bacteria were genetically encoded to respond to IBD-associated molecules by luminescing. Low-power electronic readout circuits (e.g., using nanowatt power) integrated into the device convert the light from just 1 μL of bacterial culture into a wireless signal. Biosensor monitoring was demonstrated in the GI tract of small and large animal models and integration of all components into a sub-1.4 cm³ ingestible form factor capable of supporting wireless communication. The wireless detection of short-lived, disease-associated molecules may support earlier diagnosis of disease than is currently possible, more accurate tracking of disease progression, and more timely communication between patient and their care team supporting remote personalized care.

### RELATED APPLICATIONS

This application claims priority under 35 U.S.C. § 119(e) to U.S. Provisional Application No. 63/179,804, filed Apr. 26, 2021, and entitled “ZERO-CROSSING-BASED BIO-ENGINEERED SENSOR,” to U.S. Provisional Application No. 63/255,144, filed Oct. 13, 2021, and entitled “REAL-TIME DETECTION OF LABILE BIOMARKER FOR MONITORING GUT INFLAMMATION,” and to U.S. Provisional Application No. 63/310,052, filed Feb. 14, 2022, and entitled “ARTICLE AND METHOD FOR DETECTION IN SITU,” which are incorporated herein by reference in their entireties for all purposes.

### GOVERNMENT SPONSORSHIP

This invention was made with government support under NNX16A069A awarded by the National Aeronautics and Space Administration. The government has certain rights in the invention.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Apr. 26, 2022, is named M092570922US01-SEQ-ASZ and is 25,075 bytes in size.

### TECHNICAL FIELD

Articles, systems, devices, and methods for detecting a biomarker are generally described is generally described.

### BACKGROUND

The ability to diagnose and monitor inflammatory GI disorders would be transformed if one could profile labile, oxidation-related biomarkers and their responses to dietary change and therapies in situ. Many microbiome-related conditions, notably inflammatory bowel disease (IBD), are associated with chronic intestinal inflammation resulting from dysregulated immune homeostasis, specifically, increased oxidation. Malnutrition, antibiotic resistance, antibiotic dysbiosis, neurodegenerative diseases, and mitochondrial genetic disorders are also associated with redox imbalance in the GI tract, and poor responses to chemotherapy and vaccines, as well as aging, may also be underpinned by oxidative stress.

While the etiology of IBD is not well defined, bacterial infections and antibiotics may substantially increase concentrations of oxidants, such as reactive oxygen and reactive nitrogen species (ROS/RNS). These molecules may be labile, which may make it difficult to detect their presence or accurately measure their concentration in the body. While there have been some reports of devices that sense labile molecules in the GI tract (e.g. oxidizing gases, volatile organic compounds), they are limited to off-the-shelf sensors that use non-specific metal-oxide sensing elements. Thus, improved devices, systems, and methods are desired.

### SUMMARY

Articles, systems, devices, and methods for detecting a biomarker are generally described. The subject matter of the present disclosure involves, in some cases, interrelated products, alternative solutions to a particular problem, and/or a plurality of different uses of one or more systems and/or articles.

In one aspect, a system configured for gastrointestinal monitoring and/or detection of inflammatory biomarkers is described, the system comprising a capsule; a tiered arrangement of components disposed within the capsule; the tiered arrangement of components, comprising: a porous membrane; a plurality of sealable chambers; a plurality of photodetectors, each photodetector associated with a different sealable chamber; and a power source, wherein at least one of the chambers of the plurality of sealable chambers is sized and adapted to contain a bacterial biosensor, and wherein a cross-sectional dimension of the capsule is less than or equal to 15 mm.

In another aspect, a system configured for gastrointestinal sample monitoring and/or detection of inflammatory biomarkers from a sample is described, the system comprising a capsule; a porous membrane configured to receive at least one component of the sample such that the component of the sample passes through the porous membrane; a plurality of sealable chambers; a plurality of photodetectors, each photodetector associated with a different sealable chamber; and a power source, wherein the plurality of photodetectors are integrated onto a single microelectronic chip, and wherein at least one of the chambers of the plurality of sealable chambers is sized and adapted to contain a biosensor, wherein the system is configured to provide greater than or equal to 0.1 fA of photocurrent detection using less than or equal to 5 mW of power.

In another aspect, a system configured for gastrointestinal monitoring and/or detection of inflammatory biomarkers in a subject is described, the system comprising a capsule sized and adapted for oral administration to the subject; a porous membrane disposed within the capsule; a plurality of sealable chambers, a plurality of photodetectors, each photodetector associated with a different sealable chamber; a power source associated with the plurality of photodetectors; and a plurality of bacterial and/or enzymatic biosensors configured for non-blood-based detection of a gastrointestinal inflammatory process and/or disease state of the subject, wherein each photodetector is associated with a different bacterial and/or enzymatic biosensor, such that a positive signal detected by two or more of the biosensors is correlated with the gastrointestinal inflammatory process and/or the disease state of a subject.

Other advantages and novel features of the present disclosure will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1A is a schematic diagram of a device that can detect a biomarker, according to some embodiments;

FIG. 1B is a schematic diagram of a device that can detect a biomarker and process and transmit data, according to some embodiments;

FIG. 2 is a schematic of an ingestible bio-engineered sensor capsule at mm scale for detecting target labile biomarkers in the GI tract, according to some embodiments;

FIG. 3 is a schematic diagram of a threshold-based bioluminescence detector IC with a CMOS-integrated photodiode array and a sleep/wake activation through a dual-duty-cycle front end, according to some embodiments;

FIG. 4A shows a schematic cross section of p+/n-well/p-sub photodiode in standard CMOS technology and FIG. 4B shows the equivalent circuit model for the photodiode, according to some embodiments;

FIGS. 5A-5D are diagrams showing operational phases of the threshold-based bioluminescence detector: (a) Reset, (b) Sampling, (c) Charge Transfer, and (d) Output and Discharge, according to one set of embodiments;

FIG. 6 is a schematic diagram of a programmable discharging current source implementation, according to some embodiments;

FIG. 7 is a schematic diagram of gate-leakage-based three-phase oscillators: (a) Wake-up oscillator and (b) Control oscillator; (c) Dual-duty-cycling timing diagram of the threshold-based bioluminescence detector, according to one set of embodiments;

FIGS. 8A-8C are schematic diagrams showing circuit blocks of the time-to-digital converter: (a) Current-starved ring oscillator; (b) 25%-duty-cycle clock generator; (c) StrongArm-latch dynamic comparator, according to some embodiments;

FIG. 9 is a schematic diagram of time-to-digital converter input and output signal waveforms for an example detection scenario when the photocurrent IPD1 is smaller than IPD2 for channel 0, according to some embodiments;

FIG. 10 is a schematic diagram showing both HP and LP LDOs with their current sources had similar architectures, according to some embodiments;

FIG. 11 shows a chip micrograph as well as a printed circuit boards (PCB) hosting an ingestible multi-diagnostic capsule system in a mm-scale form factor, according to some embodiments;

FIG. 12A is a schematic diagram for performing optical characterizations, according to some embodiments;

FIGS. 12B-12C show plots of measure photocurrent, according to one set of embodiments;

FIGS. 13A-13B show a schematic of a setup for in vitro measurements, and a plot of some in vitro data, according to some embodiments;

FIG. 14 shows a comparison of devices prepared with other devices, according to some embodiments;

FIG. 15 is a schematic diagram illustration a general platform for developing an ingestible capsule for real-time detection of labile mediators of disease where a probiotic bacteria are engineered to respond to an array of IBD-biomarkers (BM), and a recombinase-based genetic memory system is used to validate the bacterial biosensors in animal models, and biosensing bacteria (BS) are then re-engineered to respond by luminescing and packaged in an ingestible capsule along with miniaturized electronics (photo shows the design and dimension of our fabricated device), and while in transit through the intestines of patients, the biosensing bacteria can sense the metabolites as they are being produced in the body and the integrated ingestible capsule can transmit the bacterial luminescence signal wirelessly to an external device (e.g., a cellular phone), and the device enables remote detection for immediate follow-up after therapy (the clock), as well as monitoring the gut chemical environment for longer-term treatment with personalized therapies or dietary and lifestyle changes (calendar), and microbiome dynamics are depicted at the bottom right, as a simplified model. Starting from infancy, the microbiome, gradually reaches one of several adult states, characterized by health or disease, and monitoring the gut chemical environment is essential for timely treatments, since either perturbations that would lead to an unhealthy state can be resisted (blocked arrow) or the effective treatment plan can be assessed to return to a healthy state, according to one set of embodiments;

FIGS. 16A-16E illustrate the validation in vitro and in vivo of probiotic bacteria engineered to detect nitric oxide (NO) where FIG. 16A shows how to sense NO, the transcription factor NorR, which activates the PnorV promoter, was constitutively expressed from a low copy number plasmid (LCP, left panel), and in response to NO, NorR binds to the PnorV promoter, activating the transcription of bxb1 recombinase, leading to inversion of the GFP expression cassette (located between attB and attP sites, triangles, in a bacterial artificial chromosome, BAC) and expression of GFP, and flow cytometry was used to determine the percent of GFP-positive cells at various NO concentrations (right panel), and other reactive nitrogen species (NOx) did not activate the system, and FIG. 16B shows the NO biosensor was evaluated for its use as an NO disease stage detector: NorR, constitutively expressed from a library of ribosome-binding sites (RBS), exhibited different NO activation thresholds, and selected NO sensors detected three concentrations (15, 30, and 80 uM), which could correspond respectively to mild, moderate, and severe states of inflammation, and based on the recombinase system described in (A), flow cytometry was used to measure the percentage of GFP-positive cells at different concentrations of NO (right panel), and for each point, the mean of three biological replicates, each with n=10,000 flow cytometry events is plotted, and the error bars are the standard error of the mean (SEM), and where FIG. 16C shows the NO biosensor was validated in murine models of chemically-induced colitis, where experimental timeline (left panel): C57BL/6 male mice were treated for 7 days with 3% DSS in drinking water and gavaged every other day with engineered E. coli carrying the NO memory system, and stool was collected 6 hours post-gavage, and control mice (no DSS treatment) were simultaneously gavaged with the same engineered bacterial strain, and the sensor for NO was significantly activated on day 6 of DSS treatment (right panel), and eight-week-old C57BL/6 male mice were used for this study. “DSS” samples, n=10, “Control” samples, n=10. ***p<0.001, Student's t-test, and where FIG. 16D shows the NO sensor was validated in pigs, and the experimental design (inset, left panel): intestines were clamped to separate the different compartments to test control vs treated compartment, and bacterial sensors were placed in the different compartments (left panel), and NO Sensor 1 was significantly activated in the presence of 0.6 mM DETA-NO, and NO Sensor 3 only detected 6 mM DETA-NO, and NO Sensor 2, could detect the intermediate range, from 0.6 to 6 mM. FIG. 16E. Ischemia/Reperfusion model of inflammation in pigs. For (FIG. 16D-FIG. 16E) the bacteria were collected from the intestine after two hours of exposure to the analyte or the ischemia/reperfusion, and the percent of GFP-positive cells was measured by flow cytometry (n=10,000 events) from separately grown culture post retrieval, and lines represent the mean of these replicates and error bars are the SEM, and here, the data shows of three independent experiments (three animals [H, M, K] on different days, multiple compartments per animal). *p<0.5, **p<0.01, ***p<0.001, Student's t test, according to one set of embodiments;

FIGS. 17A-17E illustrate the design and in vitro characterization of the device for miniaturized wireless sensing with cell-based biosensors, where FIG. 17A shows the basic components and dimensions of the device, and where FIG. 17B shows the design of a miniaturized pill casing with a bacterial-electronic chamber interface with photos showing: (top) side view of the device; (bottom) fully assembled pill with the permeable membrane attached, and the bacterial chamber/casing unibody design uses a thin clear backing film to place the bacteria as close to the photosensitive electronic chip as possible, and a double-sided adhesive film enables a low-profile seal to the permeable outer filter membrane with scale bar=5 mm and where FIG. 17C shows the outputs of multiple biosensors (BS) intended to be studied together can be measured with this array (for example, the H₂O₂, TS, and TT sensors), and the detailed schematic of microelectronics PCB is shown in FIG. 28, and a threshold-based bioluminescence detector with a CMOS-integrated photodiode array was used to detect bacterial sensor output, and where FIG. 17D shows a genetic circuit and kinetic response of NO sensors in bacterial growth media supplemented with 3 mM NO; RLU, relative luminescence units, where error bars represent SEM of three independent biological replicates, and where FIG. 17E shows a wireless signal over time from the TT sensor encapsulated in the device and immersed in bacterial growth media supplemented with 100 mM TT, and low-power CMOS-integrated photodiodes converted bioluminescence emitted from the bacterial sensor into a photocurrent, which was converted into quantifiable digital data and transmitted wirelessly to the external device where lines represent the mean, and error bars denote the SEM for three independent replicates conducted with one induced device (TT) and one uninduced device (buffer), according to some embodiments;

FIG. 18A shows validation of the whole integrated device for miniaturized wireless biosensing in pigs where a schematic of the experimental flow is shown, the bacterial biosensors encapsulated in the device were tested in situ in a terminal procedure in swine, for which anesthetized Yorkshire pigs of around 90 kg were used, and after opening the abdominal cavity, the device was placed through a small incision directly into the lumen of the pig's small intestine, compartmentalized with clamps, and compartmentalized intestines were kept inside the abdomen, at 37° C., and where wireless signals transmitted from inside the abdomen were detected by a commercial receiver, which is connected to a device, such as a laptop computer or, alternatively by a cellular phone, and also showing a schematic diagram of compartmentalization of a section of the intestine clamped for experimentation, where (1) TT (100 mM) was injected with a syringe into the clamped intestinal compartment. Buffer was added as a control, in another compartment, according to one set of embodiments;

FIGS. 18B-18C show validation of the whole integrated device for miniaturized wireless biosensing in pigs showing the kinetics of TT sensor embedded in the abdominal cavity of the pig, and the response of the device placed in the compartment with TT was clearly distinguishable from that of the device in the compartment with the buffer control, and the darker lines represent the mean of independent experiments in different pigs with up to two devices in separate compartments (+TT and +buffer). Shading represents the SEM. “TT” measurements, n=3 and “Buffer” measurements, n=2. *P<0.05, Student's t test; Rel. photocurrent, Relative Photocurrent, and also showing a receiver operating characteristic (ROC) of the device sensing over time. Perfect detection is achieved at t=60 min, according to some embodiments;

FIG. 19 is a schematic diagram illustration inflammatory bowel disease (IBD) is mediated by labile molecules that are not detectable with current technologies, and following an inflammatory insult, disproportionate mucosal immune responses via cytokine signaling lead to the release of redox-active molecules such as reactive oxygen species (ROS) and nitric oxide (NO), and the resulting oxidative stress inhibits microbial growth in the gut lumen; however, chronic intestinal inflammation damages the epithelium and destroys the epithelial barrier, allowing intestinal microbes to invade the mucosa, and the sources of TS in the GI tract are mucin-derived cysteine and sulfate, which are metabolized to H₂S, and during ulceration, epithelial cells and red blood cells enter the colon; these cells produce enzymes that convert H₂S to TS. In the presence of ROS, TS is oxidized to TT, and the consumption of TT and sulfate allows certain pathogens to establish a foot-hold for infection, evoking further immune responses, and these mediators of disease are labile and cannot be measured with existing technology, and with only the limited information current approaches provide, breaking this positive feedback loop is challenging, according to some embodiments;

FIGS. 20A-20H show genetic circuit optimization and characterization of incorporated recombinase-based switch with D=dose-response curves of NO-sensing genetic circuits in E. coli Nissle, where FIG. 20A shows the translational initiation strength of the recombinase Bxb1 was varied by using different computationally designed ribosome binding sites (RBS) and the predicted RBS strengths are listed in the inset, and the lower RBS strength led to a higher SNR, and where FIG. 20B shows the memory circuit in the three NO sensors was stable over multiple rounds of re-growth, and the engineered bacteria collected in stool were cultured in a selective media to measure NO detection, and the memory system was validated to ascertain that it accurately reflected, over multiple rounds of culturing, the initial input, and where FIG. 20C shows the GFP expression did not affect growth of bacteria when ON vs. OFF states were compared, and where FIG. 20D shows the NO detection in anaerobiosis, and where FIGS. 20E-20H show the time course of switch activation, and the recombinase system triggered GFP expression within minutes (5 mins for sensor NO Sensor 1 and NO Sensor 2, 10 mins for NO Sensor 3 and less than 1 min for the ROS sensor) of exposure to the target molecule, and where the lines represent the mean, and where error bars represent the SEM of two or three independent biological replicates derived from flow cytometry experiments, each of which involved n=10,000 events, according to one set of embodiments;

FIGS. 21A-21C show plots for multiple specific disease biomarkers detected in vitro and in vivo, where the bacterial sensors were validated for the ROS H₂O₂, TS, and TT in vitro and in vivo, where in the presence of H₂O₂, the transcription factor OxyR is oxidized and activated in E. coli, and to construct a ROS biosensor that detected H₂O₂, the recombinase gene bxb1 was placed under the control of the OxyR-regulated oxyS promoter, oxySp, on the same genetic circuit, and to construct the TT and TS sensors, the oxygen repression that can affect Fumarate and Nitrate Reductase Regulator (FNR)-dependent sensors was sought to be overcome and because oxygen levels fluctuate in the gut, depending on the level of disruption of the mucosal epithelium, and to avoid this cross-repression, two newly identified sensors were used to express the recombinase system for detecting TS and TT: a TT sensor from Shewanella baltica, which does not depend on the FNR system, and the ThsRS sensor from Shewanella halifaxensis, the only genetically encoded TS sensor characterized so far, and both sensors distinguished their target molecules from other terminal electron acceptors in vitro, the lines represent the mean, the errors (SEM) are derived from flow cytometry experiments of three representative biological replicates, each of which involved n=10,000 events, and where individual points represent independent biological replicates, and the bars (days 14, 10 and 6, respectively, and the lines show the mean with SEM. *p<0.5, **p<0.01, ***p<0.001, two-way ANOVA for multiple comparisons, according to one set of embodiments;

FIGS. 22A-22E show in vivo validation of inflammatory biosensors, where FIG. 22A shows the detection of NO by the NO biosensor as a marker of GI inflammation in vivo over time. **p<0.01, ***p<0.001, ****p<0.0001, two-way ANOVA for multiple comparisons, and where FIG. 22B shows the independent validation of the presence of inflammation in the DSS colitis model by quantifying iNOS expression during DSS treatment, weight loss, and the lipocalin-2 (LCN-2) biomarker. ****p<0.0001, Student's t test, and where FIG. 22C shows the histological scores of inflammation and necrosis, indicating the validity of the DSS model, and other indicators were observed but not quantified: bloody and loose stools, poor vigor, anal prolapse, and shortening of the colon upon dissection and gross morphological examination with lines represent the mean and error bars represent the SEM of independent biological replicates, and where FIG. 22D antibiotic-triggered redox imbalance measured by the NO sensor, NO Sensor 2 allowed us to detect an exacerbated inflammatory response after antibiotic treatment (carbenicillin and chloramphenicol) in a chronic DSS inflammation model, which implies multiple rounds of DSS treatment, the biosensor for NO shows an increase of NO expression after 4 and 20-30 days of antibiotic treatment in both healthy and DSS-treated mice, with a significant switch activation on day 9 in the DSS-treated mice and on day 29 in the chronic DSS inflammation model, especially high for mouse #3. “DSS” samples, n=5 and “Control” samples, n=5. ***p<0.001, **p<0.01, two-way ANOVA for multiple comparisons, and where FIG. 22E shows sensor validation in pigs with experimental design: intestines were clamped to separate the different compartments (control vs. treated), and bacterial sensors were placed in the different compartments (left panel). The sensors registered significant activation in the presence of their respective inducer (300 μM H₂O₂, 30 mM TS, 3 mM TT, right panel), and the bacteria were collected from the intestine after two hours of exposure to the analyte, and the percent of GFP-positive cells was measured by flow cytometry with lines representing the mean, and the errors (SEM) are derived from flow cytometry experiments of three representative biological replicates, each of which involved n=10,000 events, with the data of three independent experiments (three animals [M, U, T, K] on different days, multiple compartments per animal). **p<0.01, ***p<0.001, ****p<0.0001 Student's t test, according to one set of embodiments;

FIG. 23 is a schematic size comparison of ingestible electronics and solid dosage forms with established safety rates, where the safety of ingestible devices depends, in part, on ensuring that these devices will not damage, obstruct, or be retained in the GI tract, and the current design was built to conform with the dimensions and form factors of solid dosage forms with known safety profiles and obstruction/retention rates (PillCam, Procardia XL), and the system integration at the bacterial, electronics and pill casing level allowed a significant reduction in size compared to a previously reported prototype (>9 mL to <1.4 mL), according to one set of embodiments;

FIG. 24 is a schematic diagram of the pill casing manufacturing process, where casing blanks are 3D printed via selective laser sintering with supports only on the top and bottom face to preserve the thin wall features, where the top and bottom faces are then sanded to size, and the filter membrane is cut to size with a punch and the double-sided adhesive film is laser cut with through holes aligned to the chambers, and after pressing these outer layers together, the chambers are filled with bacterial suspensions from the inside and sealed off with a thin clear adhesive film to yield the fully sealed bacterial chamber/casing unibody with a scale bar=5 mm, according to one set of embodiments;

FIG. 25 shows the effect of membrane on analyte diffusion, where the porous membranes, when placed in feces, did not interfere with detection of the target molecules, and since fouling of the membrane by fecal matter may pose a problem, several membrane materials were screened, e.g., polyethersulfone (PES), polycarbonate (PC), and polyvinylidene fluoride (PVDF), to find which material allowed the highest diffusion of the analyte molecules across the membrane with several porous membranes were tested in Franz cells (inset on the right) in the presence of feces, and the membranes tested showed similar results, with a similar percentage of detection from the NO bacterial sensors after the analyte had passed through the membrane, and the errors (SEM) are derived from flow cytometry experiments of three representative biological replicates, each of which involved n=10,000 events, according to one set of embodiments;

FIG. 26 is a plot showing nitric oxide (NO) detection and luciferase expression in anaerobiosis, where luminescence values were measured overnight post-exposure to the inducer (NO) and normalized to the optical density of the culture, and lines represent the mean, and error bars denote the SEM for three independent biological replicates, according to one set of embodiments;

FIGS. 27A-27C illustrate the kinetic response of the inflammatory biosensors with the luciferase readout where FIG. 27A-27B show E. coli Nissle biosensors were treated with their target analytes in LB (FIG. 27A) or in simulated intestinal fluid (FIG. 27B), and the luminescence response was measured in a plate reader every 3 mins for 10 hours, and the signal-to-noise ratio (SNR) was calculated by dividing the OD600-normalized luminescence values induced by the OD600-normalized luminescence values of uninduced samples, and where FIG. 27C shows response curve of the inflammatory biosensors with the luciferase readout and the E. coli Nissle inflammatory sensor strains were treated with various concentrations of their target analytes; maximal luminescence values were measured thirty minutes to two hours post-exposure to the inducer and normalized to the optical density of the culture, and where lines represent the mean, and where error bars represent the SEM of three independent biological experiments, according to one set of embodiments;

FIG. 28 is a schematic of the ingestible capsule PCB, where the custom bioluminescence detector IC was fabricated in 65 nm CMOS technology, and the top PCB holds the custom-designed multi-channel and time-multiplexed bioluminescence detector, a 6.8 mm×2.1 mm coin-cell battery, two 220 μF decoupling capacitors, and an 8-position female connector, and the bottom PCB holds a microcontroller with an integrated transmitter, a crystal oscillator, an 8-position male connector, an antenna and other components for wireless data transmission, and the components on the top and bottom PCB communicate through the two connectors; according to one set of embodiments;

FIG. 29 shows individual replicates of TT sensing in the pig intestinal environment, where the devices with the TT sensor were deposited in the intestinal compartments and TT (100 mM, blue) or buffer alone (black) were injected after temperature stabilization (˜15 mins, 37° C.), and the readings from the device were wirelessly collected for 120 minutes following device deposition, and the dark trace represent the mean of 3 replicate measurements (3 animals on different days, 2 devices per pig, in two different compartments) and pale traces indicate the individual current values for a given device. Photocurrents are provided relative to a one-time calibration value at t=15 mins, and non-induced sensor cells (black lines) decrease their luminescence output throughout the experiment, while induced cells express higher levels of luciferase, compensating signal loss over time, and for the replicates, the response of the device placed in the compartment with TT was clearly distinguishable from that of the device in the compartment with the buffer control, according to one set of embodiments;

FIG. 30 is a comparison of light detection between different chambers. E. coli Nissle strains containing a functional biosensor circuit for TT detection (TT sensor), and E coli Nissle without the gene for luciferase (null sensor) were loaded into the device. Devices were deposited in the intestine compartments and after temperature stabilization (˜15 mins), TT (100 mM, blue) or buffer alone (black) were injected, and compartmentalized intestines were kept inside the abdomen, at 37° C., and wireless signals transmitted from inside the abdomen were collected for 120 mins to analyze the kinetic response of the devices in the abdominal cavity of the pig, and the photocurrents provided relative to a one-time calibration value at t=15 mins, and non-induced sensor cells (lines) decrease their luminescence output throughout the, while induced cells express higher levels of luciferase compensating signal loss over time, and the response of the device placed in the compartment with TT was clearly distinguishable from that of the device in the compartment with the buffer control, null cells maintain constant values throughout, and error bars denote SEM for three experiments (3 animals on different days, 2 capsules per animal), according to one set of embodiments;

FIG. 31 shows a general platform for developing an ingestible capsule for real-time detection of labile mediators of disease. Probiotic bacteria are engineered to respond to an array of IBD-associated biomolecules (BM). A recombinase-based genetic memory system allows for validating the bacterial biosensors in preclinical disease models in rodents. Successful biosensing bacteria (BS) was then re-engineered to respond by luminescing and packaged in an ingestible capsule along with miniaturized electronics. The schematic illustration describes the patient case-use: while in transit through the intestines, the biosensing bacteria can sense the metabolites as they are being produced in the body and transmit the luminescence signal wirelessly to an external device. The device affords remote detection for immediate follow-up after a therapy as well as monitoring the gut chemical environment for longer-term treatment with personalized therapeutics or dietary and lifestyle changes, according to some embodiments;

FIGS. 32A-32H show the following: FIG. 32A. Genetic memory system for nitric oxide (NO). The transcription factor NorR, which activates the PnorV promoter, was used to sense NO. The transcription factor NorR activates the PnorV promoter. In response to NO, NorR, constitutively expressed from a low copy number plasmid (LCP), activates the transcription of bxb1 recombinase from the PnorV promoter on the same LCP. Bxb1 inverts the GFP expression cassette located between inversely oriented attB and attP sites (triangles) on a bacterial artificial chromosome (BAC), thus turning on GFP expression. FIG. 32B. The percent of GFP-positive cells at different NO concentrations is measured by flow cytometry. Other reactive nitrogen species (NOx) do not activate the system. FIG. 32C-FIG. 32D. NO disease stage detector. NorR, constitutively expressed from a library of ribosome-binding sites (RBS), exhibited different NO activation thresholds. Selected NO sensors can detect 3 concentrations (15, 30, and 80 uM) corresponding, respectively to mild, moderate, and severe states of inflammation, the recording the information through the recombinase system. The percent of GFP-positive cells at various NO concentrations was measured by flow cytometry. Lines represent the mean. The errors (s.d.) are derived from flow cytometry experiments of two representative biological replicates, each of which involved n=10,000 events. FIG. 32E-FIG. 32F. Nitric Oxide biosensor validated in murine models of chemically induced colitis. FIG. 32E. Experimental timeline. C57BL/6 male mice were treated for 7 days with DSS and gavaged with engineered E. coli carrying the NO memory system. Stool was collected 6 hours post-gavage. As a control, mice were simultaneously gavaged with the same engineered bacterial strain and tested in non-treated mice. FIG. 32F. The sensor for NO showed significant activation on day 6 of DSS treatment. Eight-week-old C57BL/6 male mice were used for this study. “DSS” samples, n=10, “Control” samples, n=10. ****p<0.0001, Student's t-test. FIG. 32G-FIG. 32H. Sensors validated in pigs. FIG. 32G. Experimental design. Intestines were clamped to separate the different compartments to test control vs treated compartment. FIG. 32H. Sensors show significant activation in the presence of the target molecule. Here, the data showed a representative experiment of three independent experiments (three animals on different days, multiple compartments per animal). *p< . . . , Student's t test, according to some embodiments;

FIGS. 33A-33D show design and in vitro characterization of the device for miniaturized wireless sensing with cellular biosensors. (FIG. 33A) Basic components and dimensions and chip architecture showing the dual-duty-cycling front end using a zero-crossing-based bioluminescence detector with CMOS-integrated photodiode array. (FIG. 33B) Side and front-side photos of the device; top photo of the casing showing the permeable membrane attached and the transparent film from inside. (FIG. 33C) Genetic circuit and kinetic response of NO sensors in bacterial growth media supplemented with 1 mM NO. (FIG. 33D) Wireless signal over time from the tetrathionate sensor encapsulated in the device and immersed in bacterial growth media supplemented with tetrathionate 1 mM. Low-power CMOS-integrated photodiodes convert bioluminescence emission from the bacterial sensor into photocurrent which was converted into quantifiable digital data and transmitted wirelessly to the external device. In (33D), error bars denote the SEM for three independent replicates conducted with different IMBEDs, according to some embodiments;

FIG. 34 shows in vivo validation of integrated capsule-membrane housing. The bacterial biosensors encapsulated in the device were tested in situ in a terminal procedure in swine. After opening the abdominal cavity, we placed the device through a small incision directly into the intestine, compartmentalized with clamps. In the capsule, each position in the array deploys an engineered microbial strain designed to detect one particular biomarker, here only the tetrathionate sensor is tested on the bottom left corner of the array. Tetrathionate 1 mM was added with a syringe in the induced compartment. After passage through the membrane, the biosensors were exposed to the tetrathionate in the gut biochemical milieu. Tetrathionate sensor bacteria produced a significant signal in the treated group after 4 hours of exposure, according to some embodiments;

FIG. 35 shows inflammatory bowel disease (IBD) is mediated by labile molecules undetectable with current technologies. Following an inflammatory insult, disproportionate mucosal immune responses via cytokine signaling lead to the release of redox-active molecules such as reactive oxygen species (ROS) and nitric oxide (NO). The resulting oxidative stress inhibited microbial growth in the gut lumen. However, chronic intestinal inflammation damages the epithelium and destroys the epithelial barrier, allowing intestinal microbes to invade. Both cysteine and sulfate from host mucins can be metabolized to H₂S₈, which is rapidly converted to thiosulfate via enzymatic detoxification in epithelial cells and red blood cells that enter the colon during ulceration. In the presence of ROS, thiosulfate is oxidized to tetrathionate. Consumption of tetrathionate and sulfate allowed certain pathogens to establish a foothold for infection2823, evoking further immune responses. These mediators of disease are labile and cannot be measured with the existing technology. With only the limited information current approaches provide, breaking this positive feedback loop is challenging, according to some embodiments;

FIGS. 36A-36H show the following: FIG. 36A. Genetic circuit optimization by varying translational initiation strength of the recombinase bxb1. Dose-response curves of NO-sensing genetic circuits in E. coli Nissle. The translational initiation strength of Bxb1 was varied using different computationally designed ribosome binding sites (RBS). Predicted RBS strengths are listed in the inset. Error bars represent SEM of three independent biological replicates. Lower RBS strength led to a more digital performance of the sensor. FIG. 36B. The memory circuit in the three NO sensors is stable over multiple rounds of re-growth. Since engineered bacteria collected in stool are culture in selective media to measure NO detection, it was helpful to validate that the memory system were faithfully reflecting the initial input over multiple rounds of culturing. FIG. 36C. GFP expression did not affect growth when comparing bacteria in ON vs OFF state. FIG. 36D. Nitric oxide detection in anaerobiosis. FIGS. 36E-36H. Time course of switch activation. The recombinase system triggers GFP expression within minutes (5 mins for sensor DSS1 and DSS 2 and 10 mins for DSS3 and less than 1 min for the ROS sensor) of exposure to the target molecule, according to some embodiments;

FIGS. 37A-37B show multiple specific disease biomarkers detected in vitro and in vivo. The bacterial sensors were validated for the reactive oxygen species hydrogen peroxide, thiosulfate, and tetrathionate in vitro (FIG. 37A) and in vivo (FIG. 37B), following the same protocol as was used in FIGS. 32A-32H. Individual points represent independent biological replicates and the bars show the mean with SEM**p<0.01, ***p<0.001, Student's test, according to some embodiments;

FIGS. 38A-38D show necrosis to validate inflammation detected by biosensors with histological scoring and multiple parameters of inflammation. Detection of NO by NO biosensor as a marker of GI inflammation in vivo over time (FIG. 38A). The presence of inflammation in the colitis model was independently validated by quantifying the iNOS expression during the DSS treatment (FIG. 38A) lipocalin-2 (LCN-2) biomarker (FIG. 38B) and other parameters such as weight, bloody and loose stools, poor vigor, anal prolapse, as well as shortening of the colon upon dissection and gross morphological examination as well as a histological score of inflammation and necrosis (FIG. 38C). FIG. 38D. Antibiotic-triggered redox imbalance measured by the NO-sensor. NO DSS-Sensor 2 allowed us to detect an exacerbated inflammatory response after antibiotic treatment in a chronic DSS inflammation model, which implies multiple rounds of DSS treatment. The biosensor for nitric oxide showed an increase of NO expression after 4 and 20-30 days of antibiotic treatment in both healthy and DSS-treated mice, with a significant switch activation on day 9 in the DSS-treated mice and on day 29 in the chronic DSS inflammation model, especially high for mice #3. “DSS” samples, n=5 and “Control” samples, n=5. ***p<0.001, **p<0.01, Student's t-test, according to some embodiments;

FIG. 39 shows miniaturization of the ingestible electronics to the safest size. To minimize the risks of device retention and obstruction of the GI tract, the device was miniaturized to mm scale (13 mm by 7.5 mm). Dimensions and form factors of conventional pill-shaped and round non-deformable drug delivery systems with a known safety profile can be used as a reference point for safe dimensions for current IMBED, according to some embodiments;

FIGS. 40A-40D show effect of membrane pore size and material on bacterial retention and analyte diffusion. The membrane was important to the device as it controls the exchange of nutrients and analytes required for biosensor cell function. (FIG. 40A-FIG. 40C) Using Franz cells, a series of membrane pore sizes were screened, showing that bacterial cells were significantly retained by a size larger than the traditional 0.22 μm pore size. This larger size was chosen to improve the diffusion-driven exchange of nutrients (including oxygen) and analytes. (FIG. 40A) Migration of E. coli Nissle across membranes with different pore sizes. Scanning electron microscopy of bacteria on membranes that (FIG. 40B) prevent (0.4 μm pores) or (FIG. 40C) allow slow passage (1.2 μm pores). Arrow: bacteria in pore. (FIG. 40D) The porous membranes did not interfere with detection of the target molecules when placed in feces. Fouling of the membrane by fecal matter may pose a problem, so several membrane materials were screened—e.g., polyethersulfone (PES), polycarbonate (PC) and polyvinylidene fluoride (PVDF)—to find which performed at allowing diffusion of the analyte molecules across the membrane and multiple porous membranes were compared that were tested in Franz cells (inset from the bottom right corner) in the presence of feces. The membranes tested showed similar performance, with a similar percentage of detection from the NO bacterial sensors after the analyte passing through the different membranes, according to one set of embodiments;

FIG. 41 shows nitric oxide detection and luciferase expression in anaerobiosis. Luminescence values are measured ON post-exposure to the inducer and normalized to the optical density of the culture-I may repeat this with a better sealing of the plate, according to one set of embodiments;

FIGS. 42A-42C show the following: FIG. 42A-FIG. 42B. Kinetic response of the inflammatory biosensors with the luciferase readout. E. coli Nissle biosensors were treated with their target analytes in LB (FIG. 42A) or in simulated intestinal fluid (FIG. 42B). Luminescence response was measured in a plate reader every 3 mins for 10 hours. The signal-to-noise ratio (SNR) was calculated by dividing the OD600-normalized luminescence values induced by the OD600-normalized luminescence values of uninduced samples. Error bars represent SEM of three independent biological experiments. FIG. 42C. Response curve of the inflammatory biosensors with the luciferase readout. E. coli Nissle inflammatory sensor strains were treated with various concentrations of their target analytes and max luminescence values are measured 30 mins to 2 hs post-exposure to the inducer and normalized to the optical density of the culture. Error bars represent SEM of three independent biological replicates, according to one set of embodiments;

FIG. 43 is a schematic diagram of ingestible capsules, according to some embodiments;

FIG. 44 schematically illustrates the architecture of a bioluminescence detector, according to some embodiments;

FIG. 45 shows a detailed operation of the zero-crossing based bioluminescence, according to some embodiments;

FIG. 46 shows measured results using laboratory equipment, according to some embodiments;

FIG. 47 demonstrates in vitro testing with laboratory strain of the heme biosensor, according to some embodiments; and

FIG. 48 compares the system performance of fabricated sensors with prior bioluminescence readouts, according to some embodiments.

### DETAILED DESCRIPTION

The present disclosure describes articles, devices, systems, and methods for the detection of chemical and/or biological species, such as a biomolecule or a biomarker. The ability to detect a biomolecule or biomarker may help in diagnosing, monitoring, and/or treating a condition, such as a gastrointestinal (GI) disorder.

The GI tract and its chemical environment are generally important to health and sampling of the GI environment may provide information regarding the health of a patient or a user. Existing techniques for monitoring the GI tract include endoscopic biopsies and stool analysis, and while these methods can be used to diagnose GI disorders, these techniques are either invasive (e.g., endoscopic biopsies) or may lead to degradation of valuable (but potentially difficult to isolate) biomarkers (e.g., as determined by stool analysis). While some existing systems can provide non-invasive and real time alternatives, these electronic-only systems cannot directly monitor the chemical environment of the GI tract. Existing endoscopy systems require a monitored visit to the health provider, to a high rate of unintended retention in patients which stems from the overall system size, further limiting its applicability.

Provided herein is a device that, in some embodiments, advantageously directly monitors the chemical environment of the GI tract, providing real-time, in situ monitoring of one or more biomarkers produce along the GI tract. In some embodiments, the device can advantageously quantify a low light signal from biosensor on a low power budget (e.g., less than or equal to 1000 nW), which may, in some cases, enable a significant size reduction of components of the device as compared to traditional devices. Advantageously, this reduction in size may reduce the rate of unintended retention, allowing for the unmonitored use of the devices and systems. In some cases, the device may have the configuration of a pill or a capsule that can be administered to a subject (e.g., orally) by a user (or the subject) for in situ monitoring of a biomarker(s) within the subject, such as the GI tract of the subject. As will be described in more detail below, the device may include a biosensor (e.g., a bacterial biosensor, an enzymatic biosensor, comprising a genetic circuit) that can sense or detect the presence (or absence) of a particular biomarker and generate a signal by, for example, modifying the sequence of genetic information of the biosensor. The signal (e.g., a bioluminescent signal) may be detected by one or more photodiodes (e.g., one or more CMOS photodiodes), which may indicate the presence (or absence) of the biomarker. In some cases, a plurality of biomarkers may be detected within the same device, such that the device can report the presence (or absence) of multiple biomarkers, which may increase the multiplexing ability of the device.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein or shown in the figures.

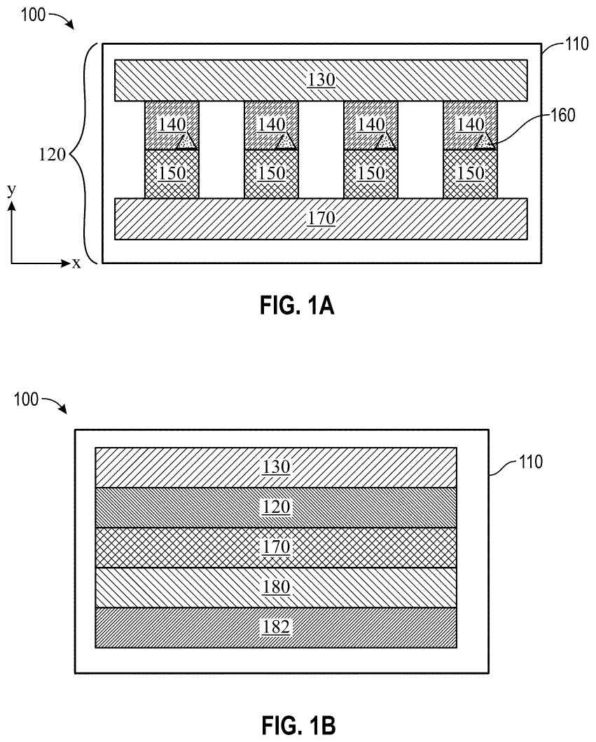

FIG. 1A is a schematic illustration of an exemplary device for detecting a biomolecule or biomarker. In some embodiments, a device 100 comprises a container e.g., in the form of a capsule 110. In some embodiments, capsule 110 comprises a tiered arrangement of components 120. In some embodiments, the tiered arrangement of components comprises a porous membrane 130, a plurality of sealable chambers 140, and a plurality of photodetectors 150. In some embodiments, each sealable chamber 140 is associated with a photodetector 150 (e.g., such that a signal generated in sealable chamber 140 is observed in the associated photodetector 150). Each sealable chamber 140 may, in some embodiments, independently comprise one or more biosensors 160. For example, as illustrated in FIG. 1A, each of the plurality of sealable chambers includes a biosensor 160. In some embodiments, each sealable chamber comprises a different biosensor. In some embodiments, two or more sealable chambers may comprise the same biosensor. Biosensors are described in more detail, below.

In some embodiments, tiered arrangement of components 120 of the device 100 further comprises a power source 170. Power sources are described in more detail, below.

While FIG. 1A shows four sealable chambers 140 and four associated photodetectors 150, those of ordinary skill in the art would understand, based upon the teachings of this specification, that any suitable number of chambers and photodetectors are possible. In some embodiments, the device comprises one or more, two or more, three or more, four or more, five or more, six or more, seven or more, eight or more, ten or more, fifteen or more, twenty or more, twenty five or more, or thirty or more sealable chambers. In some embodiments, the device comprises one or more, two or more, three or more, four or more, five or more, six or more, seven or more, eight or more, ten or more, fifteen or more, twenty or more, twenty five or more, or thirty or more photodetectors. In some embodiments, the device comprises the same number of sealable chambers and photodetectors (e.g., each sealable chamber associated with a different photodetector). In some embodiments, the device comprises different numbers of sealable chambers and photodetectors (e.g., two or more sealable chambers are associated with a single photodetector). Other combinations and numbers of components are also possible.

While FIG. 1A schematically illustrates a tiered arrangement of components including a porous membrane, a plurality of sealable chambers, a plurality of photodetectors, biosensors, and a power supply, those of ordinary skill in the art will understand, based upon the teachings of this specification, that the arrangement of components within the tier may be different than those shown in the figure, as this disclosure is not so limited. For example, the tiered arrangement of components may include a different set of components from those shown in the figure, such as an antenna and/or controller, and those skilled in the art, in view of this disclosure, will be capable of selecting the components to include in the tiered arrangement.

In some embodiments, the system or device may process and/or transmit information received from the biosensor and/or the plurality of photodiodes. The system or device may, for example, comprise a controller (e.g., a microcontroller) for processing data and/or an antenna for receiving and/or transmitting data. For example, FIG. 1B shows a device 100 comprises a controller 180 and an antenna 182, in addition to the tiered arrangement of components 120. As shown in the figure, the controller and antenna may be distinct from the tiered arrangement of components. However, in other embodiments, the controller and/or the antenna is included within the tiered arrangement of components.

As noted above, the systems and devices described herein may comprise a tiered arrangement of components. For example, the components may be stacked vertically, rather than horizontally along a plane of a substrate. In some embodiments, the components are arranged such that an imaginary axis line passes through the membrane, at least one sealable chamber, at least one photodetector, and the power source. By way of illustration (and not limitation), in FIG. 1A, the components of the tiered arrangement of components 120 are aligned along the vertically oriented y-axis rather than the horizontally oriented x-axis. Advantageously, by having tiered components, the overall size of the system or device may be reduced relative to existing devices and systems, which may allow the system or device to be ingested by a subject, for example, for in situ monitoring of the subject. In some embodiments, at least two of the tiered components are in contact with two other components. The tiered components may include any of the components of the system or device described herein, e.g., one or more porous membranes, sealable chambers, biosensors, photodetectors, power sources, as this disclosure is not so limited.

In some embodiments, one or more of the components of the tiered arrangement of components independently has a particular aspect ratio. In some embodiments, an aspect ratio of one of the components of the tiered arrangement of components is greater than or equal to 1:1, greater than or equal 1.1:1, greater than or equal to 1.2:1, greater than or equal to 1.3:1, greater than or equal to 1.5:1, greater than or equal to 2:1, greater than or equal to 5:1, greater than or equal to 10:1, greater than or equal to 20:1, greater than or equal to 50:1, greater than or equal to 100:1, greater than or equal to 1,000:1, or greater than or equal to 10,000:1. In some embodiments, an aspect ratio of one of the components of the tiered arrangement of components is less than or equal to 10:000:1, less than or equal to 1,000:1, less than or equal to 100:1, less than or equal to 50:1, less than or equal to 20:1, less than or equal to 10:1, less than or equal to 5:1, less than or equal to 2:1, less than or equal to 1.5:1, less than or equal to 1.3:1, less than or equal to 1.2:1, less than or equal to 1.1:1, or less than or equal to 1:1. Combinations of the above-referenced ranges are also possible (e.g., greater than or equal to 1:1 and less than or equal to 10,000:1). Other ranges are possible. For embodiments comprising more than one component, each component may independently have an aspect ratio within the above-referenced ranges.

Various embodiments comprise a biosensor for detecting the presence (or absence) of a chemical and/or biological species, such a biomolecule or a biomarker. In some embodiments, the biosensor is a bacterial biosensor comprising one or more bacteria, wherein the bacteria can detect the presence (or absence) of one or more chemical and/or biological species (e.g., biomarkers). In some embodiments, the bacteria comprise bioengineered bacteria, wherein the bacteria has been genetically modified (e.g., plasmid insertion) to detect the presence (or absence) of one or more chemical and/or biological species. The bacteria, whether naturally occurring or genetically modified, may undergo one or more chemical and/or biological reactions when exposed to a particular biomarker (or if the particular is removed or is not present), which may subsequently result in additional chemical and/or biological reactions. In some embodiments, the presence of a biomarker (e.g., nitric oxide, NO) may cause a first chemical and/or biological reaction. In some embodiments, the presence of the biomarker may cause the first chemical and/or biological reaction to produce a species involved in a second chemical and/or biological reaction. In some such embodiments, the second chemical and/or biological reaction includes a regulatory molecule (e.g., a protein/peptide) involved in the regulation of DNA and/or RNA, such as a promotor. In some such embodiments, the regulatory molecule may be produced from the first and/or second chemical and/or biological reaction and may then bind to a promotor to initiate transcription. This may result in the production of a specific enzyme, which may, in some cases, facilitate the generation of a signal within the bacteria.

By way of example, the first chemical and/or biological reaction can be a first biological reaction (e.g., provided to the bacteria by a plasmid) in which NO is the biomarker. The NO may bind or react with a species characteristic of the first biological reaction to produce a regulatory molecule, such as a promoter. The promoter may activate a second chemical and/or biological reaction, such as a second biological reaction, which may subsequently generate a signal, for example by activating (or deactivating) a bioluminescent molecule (e.g., green fluorescent protein, GFP). Of course, other biomarkers and chemical and/or biological reactions are possible and are described elsewhere herein.

The bacterial biosensors may be configured, in some cases, to detect a variety of biomolecules or biomarkers. In some embodiments, the bacterial biosensors are configured to detect NO. In some embodiments, the bacterial biosensors are configured to detect reactive oxygen species (ROS), such as peroxides, hydroxyl radicals, and/or superoxide. In some embodiments, the bacterial biosensors are configured to detect thiosulfate and/or tetrathionate. Other species the bacterial biosensors may be configured to detect include, but are not limited to, inflammatory markers, proteins, DNA, RNA, hormones, chemical analytes, or the like.

In some embodiments, a plurality of bacterial and/or enzymatic biosensors are configured for non-blood-based detection of a gastrointestinal inflammatory process and/or disease state of the subject. However, in some embodiments, one or more biosensors may be configured to detect one or more components or blood and/or serum.

Advantageously, the devices described herein may be configured to detect two or more different biomolecules and/or biomarkers. For example, in an illustrative set of embodiments, the detection of a single biomolecule by a first biosensor may indicate a first disease state of the subject. In some embodiments, the detection of two different biomolecules by two different biosensors may indicate a second disease state, different than the first disease state, of the subject. In some embodiments, the plurality of sealable chambers, biosensors, and photodetectors advantageously provide a combinatorial approach to disease detection and monitoring in a subject. For example, different combinations of positive results (e.g., a detectable signal produced by a biosensor) by different biosensors may be correlated with different diseases and/or disease states.

In an exemplary illustration of the above noted detection, in some embodiments, a first detectable signal produced by a first biosensor corresponds to a first disease state. In some embodiments, a second detectable signal produced by a second biosensor (different than the first biosensor) corresponds to a second disease state, different than the first disease state. In some embodiments, the presence of the first detectable signal and the second detectable signal corresponds to a third disease state, different than the first disease state and the second disease state. In some embodiments, the presence of a third detectable signal produced by a third biosensor corresponds for a fourth disease state. In some embodiments, the presence of the first detectable signal and/or the second detectable signal and the third detectable signal corresponds to a fifth disease state. Other combinations and diseases/disease states are also possible.

A variety of suitable bacteria may be suitable for bacterial biosensors. In various embodiments, the bacterial biosensor comprises Escherichia coli (e.g., E. coli, E. coli Nissle 1917 (EcN)). However, other bacteria may be suitable for the bacterial biosensors. Non-limiting examples of other suitable bacteria include Bacteroidetes, Firmicutes, Actinobacteria, Proteobacteria. Other bacteria are possible as this disclosure is not so limited.

In some embodiments, the biosensor may comprise yeast, such as Saccharomyces cerevisiae (such as S. cerevisiae boulardii) and/or other endogenous fungi, (e.g Candida albicans), without limitation. Other yeast are possible.

In some embodiments, one or more biosensors comprises an enzymatic biosensor or a non-enzymatic biosensor. An enzymatic biosensor may comprise an enzyme that recognizes a biomarker to produce an output that can be sensed by the electronic component of the device. In some embodiments, output comprises a signal generated through: 1) the enzymatic conversion of the biomarker into a new product; 2) biomarker-mediated inhibition or activation of the enzyme; or 3) biomarker-mediated modification of enzyme properties. By contrast, a non-enzymatic biosensor does not require interaction between an enzyme and a biomarker. For example, in some embodiments, a non-enzymatic biosensor may comprise a protein channel that facilitates signal flow (or output) when in the presence of an particular biomarker. In some embodiments, a non-enzymatic biosensor comprises an antibody or a binding protein that recognizes the presence of a biomarker. In some embodiments, the non-enzymatic biosensor comprises a nucleic acid that hybridizes to an analyte or otherwise binds to it (e.g., as an aptamer). In some embodiments, the non-enzymatic biosensor comprises of a transcription factor that alters gene expression upon binding to an analyte.

In some embodiments, the biosensors (e.g., bacterial biosensors) are configured to generate a luminescent signal, such a bioluminescent signal. Advantageously, a bioluminescent signal may be encoded into the biosensor so that light (or the absence thereof) may indicate the presence (or absence) of a biomarker detected by the biosensor.

In some embodiments, a system or device comprises a plurality of photodetectors. In some such embodiments, the plurality of photodetectors may be configured to detect a luminescent signal, such a bioluminescence from one or more biosensors. The plurality of photodetectors comprises a CMOS-integrated photodiode array, which may advantageously improve the ability to miniaturize the systems and devices (e.g., microscale, nanoscale miniaturization), and may also lower the power required to power such systems and devices. In some embodiments, the plurality of photodetectors are integrated onto a single microelectronic substrate (e.g., a microchip) as a component of the tiered arrangement of components.

In some embodiments, the plurality of photodetectors may include any component that converts light or other electromagnetic radiation into an electrical signal (e.g., current, voltage). Non-limiting examples of suitable photodetectors include phototransistors and photodiodes. These include charge-coupled device (CCD) arrays and complementary metal oxide semiconductor (CMOS) arrays. Advantageously, because many electronic devices (e.g., smartphones) generally leverage CMOS-based photodetectors to detect light, the use of CMOS-integrated photodetectors may improve the ease of fabrication of the systems and devices described herein.

In some embodiments, a controller (e.g., a microcontroller) is configured to receive data (e.g., an electrical current providing the data) from at least one of the photodetectors (e.g., a photodiode) of the plurality of photodetectors. For example, the system or device may comprise an element (e.g., a circuit) configured to convert light information from a photodetector into digital counts which may be provide to the controller and/or wireless transmitter. In some embodiments, the controller is configured to receive at least 2, at least 3, at least 4 separate pieces of data from each of the photodetectors to provide multiple data points. Advantageously, when the systems and devices are used to diagnosis and/or monitor a disease or condition, it may be helpful to detect multiple biomarkers that may be related to the disease or condition, and by providing data from multiple biosensors (e.g., as detected by two or more of photodetectors of the plurality of photodetectors), the disease or condition may be more accurately monitored. By way of illustration (and not limitation), a condition may be associated with a first biomarker and a second biomarker, and some embodiments described herein may detect both the first biomarker and the second biomarker in order to provide information about the condition. Of course, in other embodiments, only the first biomarker is detected, as this disclosure is not so limited.

In some embodiments, the systems and devices comprises wireless capabilities for enabling suitable communication with other devices/systems (e.g., transmitting information to a user, such as the presence or absence of a biomarker). Wireless devices are generally known in the art and may include, in some cases, LTE, WiFi and/or Bluetooth systems. In some embodiments, the systems and devices described herein comprise such a wireless device.

In some embodiments, the systems and devices may be configured to adjust various parameters based on physiological and/or external metrics. For example, in some embodiments, the systems and devices are configured to adjust the rate and/or amount a first parameter in response to a signal from a sensor in electrical or wireless communication with and/or associated with the system or device. In some embodiments, the systems and devices adjusts the first parameter in response to an input from the user and/or a signal from the sensor (e.g., the biosensor).

Non-limiting examples of other suitable sensors for use systems and devices described herein include temperature sensors (e.g., monitoring internal temperature, ambient temperature, temperature of a component associated with the system or device, such as a thermally sensitive polymer), physiological/biometric sensors (e.g., heart rate, electrical activity, neuronal activity), accelerometers (e.g., for measuring breathing rate, activity levels, sleeping behavior/patterns), and environmental sensors (e.g., pH, biologic concentration, chemical concentration).

The systems and devices described herein may be implemented by any suitable type of analog and/or digital circuitry. For example, the systems and devices may be implemented using hardware or a combination of hardware and software. When implemented using software, suitable software code can be executed on any suitable processor (e.g., a microprocessor) or collection of processors. The systems and devices may be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein may, in some cases, comprise at least one computer-readable storage medium (e.g., RAM, ROM, EEPROM, flash memory or other memory technology, or other tangible, non-transitory computer-readable storage medium) encoded with a computer program (i.e., a plurality of executable instructions) that, when executed on one or more processors, performs the above-discussed functions of one or more embodiments. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

Various systems and devices described herein comprise a plurality of sealable chambers. Each sealable chamber may independently comprise one or more biosensors and/or enzymatic sensors are described above and elsewhere herein. The sealable chamber may reduce or prevent undesired exposure of the one or more biosensors to moisture or other liquids. In some embodiments, each photodetector is associated with a different sealable chamber. In some such embodiments, this may advantageously provide each photodetector to receive a different signal from each sealable chamber (e.g., one or more biosensors within the sealable chamber). In some embodiments, at least one of the plurality of sealable chambers is sized and adapted to contain a bacterial biosensor.

Each chamber of the plurality of sealable chambers may independently be sized and shaped with a particular dimension. In some embodiments, at least some of the chambers of the plurality of sealable chambers has a volume of greater than or equal to 0.1 μL, greater than or equal to 0.2 μL, greater than or equal to 0.3 μL, greater than or equal to 0.4 μL, greater than or equal to 0.5 μL, greater than or equal to 0.7 μL, greater than or equal to 1.0 μL, greater than or equal to 2 μL, greater than or equal to 3 μL, greater than or equal to 4 μL, or greater than or equal to 5 μL. In some embodiments, at least some of the chambers of the plurality of sealable chambers has a volume of less than or equal to 5 μL, less than or equal to 4 μL, less than or equal to 3 μL, less than or equal to 2 μL, less than or equal to 1 μL, less than or equal to 0.7 μL, less than or equal to 0.5 μL, less than or equal to 0.4 μL, less than or equal to 0.3 μL, less than or equal to 0.2 μL, or less than or equal to 0.1 μL. Combinations of the above-referenced ranges are also possible (e.g., greater than or equal to 0.1 μL and less than or equal to 5 μL). Advantageously, a relatively small sized chamber (e.g., less than or equal to 5 μL may lower the power requirements of the system or device. Of course, other ranges are possible as this disclosure is not so limited. In some embodiments, some of the chambers of the plurality of chambers has a volume within the above-referenced ranges. In some embodiments, all of the chambers has a volume within the above-referenced ranges.

In some embodiments, the system or device may comprise a porous membrane. The porous membrane may be configured to receive at least one component of a sample such that the component of the sample passes through the porous membrane.

The porous membrane may have pores of any suitable size. In some embodiments, an average pore diameter of the pores is greater than or equal to 0.1 μm, greater than or equal to 0.2 μm, greater than or equal to 0.5 μm, or greater than or equal to 1.0 μm. In some embodiments, an average pore diameter of the pores is less than or equal to 1.0 μm, less than or equal to 0.5 μm, less than or equal to 0.2 μm, or less than or equal to 0.1 μm. Combinations of the above-referenced ranges are also possible (e.g., greater than or equal to 0.2 μm and less than or equal to 1.0 μm). Other ranges are possible.

In some embodiments, the systems and devices also include a power source. The power source may be any suitable source of power, such as a battery. In some embodiments, the power source provides a relatively low amount of power (e.g., less than or equal to 1000 nW of power.

As noted above, in some embodiments, the systems and devices are associated with and/or comprises a power source. The power source may include any appropriate material(s), such as one or more batteries, photovoltaic cells, etc. Non-limiting examples of suitable batteries include Li-polymer (e.g., with between 100 and 1000 mAh of battery life), Li-ion, nickel cadmium, nickel metal hydride, silver oxide, or the like. In some cases, the battery may apply a voltage in response to a physiological and/or external metric and/or signal (e.g., by a user). For example, the average magnitude of the voltage applied may be between 0.001 to 0.01 V, between 0.01 to 0.1 V, between 0.1 V and 10.0 V, between 1.0 V and 8.0 V, between 2.0 V and 5.0 V, between 0.1 V and 5.0 V, between 0.1 V and 1.5 V, between 0.1 V and 1.0 V, between 1.0 V and 3.0 V, between 3.0 V and 8.0 V, or any other appropriate range.

In some embodiments, the devices and systems require relatively low power during operation. For example, in some embodiments, a device or system is configured to provide greater than or equal to 0.1 fA of photocurrent detection using less than or equal to 1000 nW of power. In some embodiments, a device or system is configured to provide greater than or equal to 0.1 fA, greater than or equal to 0.5 fA, greater than or equal to 1 fA, greater than or equal to 10 fA, greater than or equal to 100 fA, greater than or equal to 250 fA, greater than or equal to 500 fA, greater than or equal to 750 fA, greater than or equal to 1000 fA, greater than or equal to 5 pA, greater than or equal to 10 pA, greater than or equal to 20 pA, greater than or equal to 50 pA, or greater than or equal to 100 pA of photocurrent detection while using less than or equal to 5 mW of power, less than or equal to 1 mW of power, less than or equal to 500 μW of power, less than or equal to 100 μW of power, less than or equal to 50 μW of power, or less than or equal to 10 μW of power, or less than or equal to 1000 nW of power. In some embodiments, a device or system is configured to provided less than or equal to 100 pA, less than or equal to 50 pA, less than or equal to 20 pA, less than or equal to 10 pA, less than or equal to 5 pA, less than or equal to 1000 fA, less than or equal to 750 fA, less than or equal to 500 fA, less than or equal to 250 fA, less than or equal to 100 fA, less than or equal to 1 fA, less than or equal to 0.5 fA, or less than or equal to 0.1 fA while using greater than or equal to 1000 nW of power, greater than or equal to 10 μW of power, greater than or equal to 50 μW of power, greater than or equal to 100 μW of power, greater than or equal to 500 μW of power, greater than or equal to 1 mW of power, or greater than or equal to 5 mW of power. Combinations of the above-referenced ranges are possible (e.g., providing 0.1 fA of current while using less than or equal to 5 mW of power). Other ranges are possible.

In some embodiments, the system or device is configured to consume less than or equal to 20 mW, less than or equal to 15 mW, less than or equal to 10 mW, less than or equal to 1 mW, less than or equal to 500 μW, less than or equal to 100 μW, less than or equal to 50 μW, less than or equal to 10 μW, less than or equal to 1000 nW, less than or equal to 500 nW, or less than or equal to 100 nW. In some embodiments, the system is configured to consume greater than or equal to 100 nW, greater than or equal to 500 nW, greater than or equal to, 1000 nW, greater than or equal to 10 μW, greater than or equal to 50 μW, greater than or equal to 100 μW, greater than or equal to 500 μW, greater than or equal to 1 mW, greater than or equal to 15 mW, or greater than or equal to 20 mW. Combinations of the above-referenced ranges are also possible (e.g., greater than or equal to 100 nW and less than or equal to 20 mW). Other ranges are possible.

The systems and devices described herein may be housed in a pill or capsule configuration. Such a configuration may advantageously facilitate consumption of the systems and devices for internal or in situ monitoring of a user or subject. In some embodiments, a tiered arrangement of components is disposed within the capsule.

For embodiments including a capsule, the capsule may have any suitable dimensions. In some embodiments, an average cross-sectional dimension of the capsule is greater than or equal to 0.1 mm, greater than or equal to 0.5 mm, greater than or equal to 1 mm, greater than or equal to 2 mm, greater than or equal to 3 mm, greater than or equal to 4 mm, greater than or equal to 5 mm, greater than or equal to 7 mm, greater than or equal to 10 mm, greater than or equal to 12 mm, or greater than or equal to 15 mm. In some embodiments, an average cross-sectional dimension of the capsule is less than or equal to 15 mm, less than or equal to 12 mm, less than or equal to 10 mm, less than or equal to 7 mm, less than or equal to 5 mm, less than or equal to 4 mm, less than or equal to 3 mm, less than or equal to 2 mm, less than or equal to 1 mm, less than or equal to 0.5 mm, or less than or equal to 0.1 mm. Combinations of the above-referenced ranges are also possible (e.g., greater than or equal to 0.1 mm and less than or equal to 15 mm). Other ranges are possible.

As noted above, in some embodiments, the systems and devices described herein are configured to be enclosed within a capsule. In some embodiments, the capsule is a 000 capsule or smaller (e.g., the capsule has a shape or size as described in the USP including, but not limited to, 000 capsule, 00 capsule, 0 capsule, 1 capsule, 2 capsule, 3 capsule, 4 capsule, or 5 capsule.) In some embodiments, the capsule at least partially encapsulates a first portion and/or a second portion of the system or device. In some embodiments, multiple systems and/or devices can be placed inside of a capsule.

In some embodiments, although the systems or devices may be configured for potential encapsulation in a 000 capsule, or smaller, the systems and devices not necessarily need to be encapsulated in such capsule. In embodiments wherein the systems and devices are to be administered, such as by ingesting the system or device, the system or device may thus be administered without encapsulation.

In some embodiments, the system or device may include a coating on at least a portion of an outer surface of the system or device (e.g., on a portion of an outer surface of a capsule). In some embodiments, the systems and devices comprises a coating (e.g., a film disposed on a least a surface of the system). In some embodiments, the coating may be applied as an aqueous or organic solvent-based polymer system, fats and/or wax. In some embodiments, the coating comprises one or more of a polymer, a plasticizer, a colorant, a solvent, a fat, and a wax. Non-limiting examples of suitable fats and/or waxes include beeswax, carnauba wax, cetyl alcohol, and cetostearyl alcohol.

Non-limiting examples of suitable polymers for the coating include of cellulosic (e.g. hydroxypropylmethylcellulose, hydroxypropylcellulose, hydroxyethylcellulose, hydroxyethylcellulose phthalate, ethylcellulose, cellulose acetate phthalate, cellulose acetate trimellitate), vinyl (e.g. poly(vinyl pyrrolidone), poly(vinyl alcohol), poly(vinyl pyrrolidone)-poly(vinyl acetate)copolymers, poly(vinyl alcohol)-poly(ethylene glycol) co-polymers, poly(vinyl acetate phthalate), glycols (e.g. poly(ethylene glycol)), acrylics (e.g. amino alkyl methacrylate copolymers), other carbohydrates (e.g. maltodextrin, polydextrose), and combinations thereof.

Non-limiting examples of suitable colorants include natural pigments (e.g. riboflavin, beta-carotene, carmine lake), inorganic pigments (e.g. titanium dioxide, iron oxides), water-soluble dyes (FD&C Yellow #5, FD&C blue #2), FD&C lakes (FD&C Yellow #5 Lake, FD&C Blue #2 Lake), and D&C lakes (D&C Yellow #10 Lake, D&C Red #30 Lake).

Non-limiting examples of suitable plasticizers include polyhydric alcohols (e.g. propylene glycol, glycerol, polyethylene glycols), acetate esters (e.g. triacetin, triethyl citrate, acetyl triethyl citrate), phthalate esters (e.g. diethyl phthalate), glycerides (e.g. acylated monoglycerides) and oils (e.g. castor oils, mineral oils).

Polymers, plasticizers, colorants, solvents, fats, and/or waxes may be combined in any suitable amount to form the coating. The coating may be applied in any suitable method including, for example, dip coating and/or spray atomization. Other methods of depositing the coating are also possible.

The articles, devices, systems, and method described herein are suitable for a variety of application. As mentioned above, inventive devices (e.g., capsules) may be used in a variety of ways, such as disease monitoring. For example, capsules may be used internally within a subject to monitor a condition, such as the condition of the GI tract, and may also determine specific conditions or diseases of the GI tract, such as IBS or Crohn's disease. In some embodiments, the systems and devices are configured to detect NO, ROS, and/or hydrogen sulfide (e.g., hydrogen sulfide may be detected indirectly by determining a related biomolecule, such as thiosulfate and/or tetrathionate). In some embodiments, a biomarker may be related to blood, such as a heme-comprising biomarker; however, it should be understood that embodiments described herein are not limited to blood-detecting systems or devices, as other biomarkers can be detected. In some cases, the articles, systems, devices, and methods described herein may to be used to monitor non-disease physiological biomolecules related to health, for example, a device (e.g., a capsule) that monitors pH levels (e.g., H₃O⁺, OH⁻), stress molecules (e.g., cortisol), immune molecules (e.g., the presence or absence of certain antibodies), diet molecules, sleep molecules, in addition to the amount of a particular drug level in a particular portion of the body (e.g., the GI tract, the cardiovascular system). Other applications are possible.

According to some embodiments, the articles, devices, and systems described herein are compatible with one or more therapeutic, diagnostic, and/or enhancement agents, such as drugs, nutrients, microorganisms, in vivo sensors, and tracers.

The articles, devices, systems, and methods may be useful for a variety of purposes, including diagnostic purposes, on a variety of subjects. The term “subject,” refers to an individual organism such as a human or an animal. In some embodiments, the subject is a mammal (e.g., a human, a non-human primate, or a non-human mammal), a vertebrate, a laboratory animal (e.g., a mouse, a pig), a domesticated animal, an agricultural animal, or a companion animal. In some embodiments, the subject is a human. In some embodiments, the subject is a rodent, a mouse, a rat, a hamster, a rabbit, a dog, a cat, a cow, a goat, a sheep, or a pig. Other subjects are possible as this disclosure is not so limited.

In some embodiments, the systems and devices are administered to a subject (e.g., orally). In some embodiments, the system may be administered orally, rectally, vaginally, nasally, and/or uretherally. In some embodiments, the system or device is administered to the colon, the duodenum, the ileum, the jejunum, the stomach, or the esophagus.

In some embodiments, a miniaturized, wireless, ingestible biosensor capsule is to measure in vivo biomarkers of intestinal inflammation in a subject is described, the capsule comprising a housing, at least one biosensor component, and an electric component, wherein (a) the biosensor component comprises a biosensing genetic circuit capable of detecting biomarkers of intestinal inflammation, said circuit undergoing luminescence upon detection of said biomarkers and communicating said luminescence to the electric component, and (b) the electric component comprises an electronic readout circuit to detect luminescence and low-power electronics to process the luminescence data by periodically sampling a photon-generated charge and transmitting the information wirelessly from inside of the subject's body to an external device, wherein the capsule measures no more than 15 mm in diameter and no more than 8 mm in height, optionally wherein the capsule dimensions are approximately 14.25 mm by 8.5 mm

In some embodiments, a capsule is described, wherein the intestinal inflammation biomarkers are selected from the group consisting of intestinal gases, metabolic by-products, and microbiota-related molecules.

In some embodiments, a capsule is described, wherein the intestinal inflammation biomarkers are selected from the group consisting of nitric oxide, H₂O₂, ROS, thiosulfate and tetrathionate.

In some embodiments, a capsule is described, wherein the biosensing genetic circuit comprises probiotic bacteria engineered to respond to biomarkers of intestinal inflammation.

In some embodiments, a capsule is described, further comprising memory circuits to report exposure to the biomarkers.

In some embodiments, a capsule is described, wherein the engineered sensor does not include a fumarate and nitrate reductase regulator (FNR).

In some embodiments, a capsule is described, wherein the external device is a smart phone.

In some embodiments, a capsule is described, wherein the electronic component further comprises a CMOS-integrated photodiode array, threshold-based bioluminescence detector, time-to-digital converter, voltage references, regulators, and a microcontroller that enables wireless data transmission.

In some embodiments, a capsule is described, further comprising a low-power luminometer chip.

In some embodiments, a method of monitoring the health of a subject comprising orally administering an ingestible capsule to a subject, the method comprising measuring the level of luminescence, wherein the level of luminescence above a threshold level indicates inflammation.

In some embodiments, a method of detecting the presence of IBD in a subject comprising orally administering an ingestible capsule to a subject 1 to monitor for the presence of biomarkers of intestinal inflammation and detecting IBD if biomarkers of intestinal inflammation are detected.

In some embodiments, a method is described, wherein the intestinal inflammation biomarkers are selected from the group consisting of intestinal gases, metabolic by-products, and microbiota-related molecules.

In some embodiments, a method is described, wherein the intestinal inflammation biomarkers are selected from the group consisting of nitric oxide, H₂O₂, ROS, thiosulfate and tetrathionate.

In some embodiments, a method of treating IBD in a subject comprising orally administering the ingestible capsule of claim 1 to monitor for the presence of biomarkers of intestinal inflammation and administering to the subject therapeutics for IBD if the biomarkers of intestinal inflammation are detected.

In some embodiments, a method is described, wherein the intestinal inflammation biomarkers are selected from the group consisting of intestinal gases, metabolic by-products, and microbiota-related molecules.

In some embodiments, a method is described, wherein the intestinal inflammation biomarkers are selected from the group consisting of nitric oxide, H₂O₂, ROS, thiosulfate and tetrathionate.

The following examples are intended to illustrate certain embodiments of the present invention, but do not exemplify the full scope of the invention.

### Example 1

The following example describes a system comprising a bacterial biosensor configured to detect an inflammation-related biomarker.

This example presents a system for biochemical detection using genetically engineered biosensor bacteria in a highly miniaturized ingestible capsule form factor. The core integrated circuit (IC) is a threshold-based bioluminescence detector with a CMOS-integrated photodiode array in a 65 nm technology that utilizes a dual-duty-cycling front end to achieve low power consumption. The implemented IC achieved 59 nW active power consumption, 25 fA/count resolution, and a 59 fA minimum detectable signal (MDS) using a calibrated optical source. The IC was then integrated with other system components into battery-powered wireless ingestible capsule measuring just 6.5 mm thick×12 mm diameter. Successful detection of low-intensity bioluminescent signals from bioengineered bacterial sensors was demonstrated when exposed to the intestinal inflammation biomarker, tetrathionate, at 100 mM in vitro. Together, the integrated circuit and mm-scale “smart pill” system demonstrated high sensitivity with low-power multiplexed measurement capability suitable for noninvasive disease diagnosis and monitoring in the gastrointestinal (GI) tract.

The gastrointestinal (GI) tract and its chemical environment are important health. The current gold standards for monitoring the GI tract include endoscopic biopsies and stool analysis. While these methods are used to diagnose diseases, such as inflammatory bowel disease, they are either invasive (endoscopic biopsies) or lead to degradation of valuable but labile biomarkers (e.g., determined by stool analysis). Commercial electronic diagnostic pills such as PillCam and SmartPill provide a non-invasive and real time alternative, however, these electronic-only systems cannot directly monitor the gut chemical environment. Electronic pills with cell-based sensing elements would be ideal for monitoring the GI chemical environment because intestinal microbes naturally survive and sense molecules in the GI tract with high sensitivity and specificity. Cell-based biosensors are also cost-effective because they do not require the laborious purification processes involved in protein and nucleic acid-based sensing elements. In fact, cell-based biosensors have been demonstrated in a range of point-of-care test (POCT) applications including medical diagnostics, food production, and environmental monitoring. Despite this opportunity, there are no examples of an electronic pill that incorporates cell-based biosensors for monitoring the GI tract. A key remaining obstacle is reduction of the overall system size to prevent GI tract blockage while maintaining adequate bacterial-electronic coupling. Here, this challenge is tackled by implementing a custom integrated circuit (IC) that can quantify an ultra-low light signal from biosensor bacteria on a nW power budget enabling a significant size reduction of all the key components of an integrated bacterial-electronic diagnostic pill.

Electronic-based readout elements complement the sensing features of cell-based biosensors, allowing real-time, wireless communication of the results from inside the body to consumer electronics. However, unlike biosensors based on biomolecule-functionalized electrodes, bacteria cannot be as readily coupled to electronics in a small form factor. Bacterial-electronic coupling modalities based on the absorption and/or emission of light are ideal in the harsh aqueous environment of the gut because they allow for complete galvanic isolation of the electronic components. Of these, bioluminescence provides the most volume and power-efficient option because unlike colorimetric (biosynthesis of pigments) or fluorescent (expression of fluorescent proteins) signals, it does not require a light source or optical filters. Additionally, the natural human gut environment is dark, allowing a bioluminescent signal to act as a unique marker of the cell-based biosensor. However, even optimized bioluminescent signals from bacteria tend to be very weak (≈20-200 photons/sec/cell) and have been traditionally quantified using large, power-hungry bench-top instruments (e.g., plate readers, gel imager). Therefore, an ultra-low-power electronic system capable of acquiring and encoding these weak bioluminescent signals would offer the possibility of distributed and cost-effective POCTs and disease monitoring.

Weak bioluminescent signals have been detected by previous systems, however, they are either too power-hungry or too large to be safely used as an ingestible diagnostic pill in humans. While some μW- to mW-level systems with integrated photodiodes and an operational transconductance amplifier (OTA)-based readout exhibit cell-based signal acquisition, there is a need to reduce the power consumption to reduce the battery size, and ultimately, the capsule size to improve safety. Recently, there was a successful demonstration of a nW-level amplifier system, however, the overall system used off-chip commercial phototransistors which resulted in a size that carries significant risk of intestinal blockage. Furthermore, the use of off-chip phototransistors limits the scalability to support multiplex measurements from cell-based biosensors. Therefore, there is an urgent need to develop ICs that can acquire weak bioluminescent signals using a scalable architecture capable of higher multiplexing while consuming nW power levels.

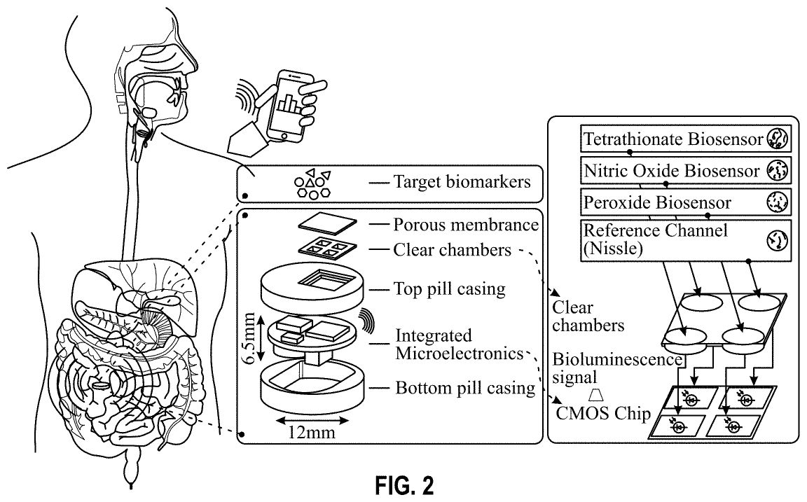

This example demonstrates a wireless ingestible capsule, composed of genetically engineered bacterial biosensors for sensing and integrated circuits for signal encoding and communication, with a total electronic system size of 6.5 mm by 12 mm, as shown in FIG. 2. One component of this capsule is a custom-designed threshold-based bioluminescence detector IC consuming nW-level active power while achieving high sensitivity and high resolution. To achieve this performance, a readout circuit architecture was developed integrating an array of photodiodes in CMOS with low-power luminescent signal processing techniques (i.e., threshold-based encoding). Together, these advances allowed detection of fA-level photocurrents from just 1 μL of bacterial cells. This power-efficient IC enabled a complete wireless ingestible capsule consuming 15.55 μW. This stringent power budget allows continuous monitoring of the GI tract for up to 37 days with a small form-factor 5.5 mAh coin-cell battery. In the future, this power level could be alternatively supplied through a battery-less system using biofuel cells to support the target function in the GI tract for 24-48 hours.

This example is organized as follows. Section II presents the overall system architecture and key features. Section III discusses the circuit-level implementation and analysis of the threshold-crossing-based bioluminescence detector. The measurement setup and results are presented in Section IV, followed by the conclusions in Section V.

### II. Multi-Channel and Time-Multiplexed Bioluminescence Detector

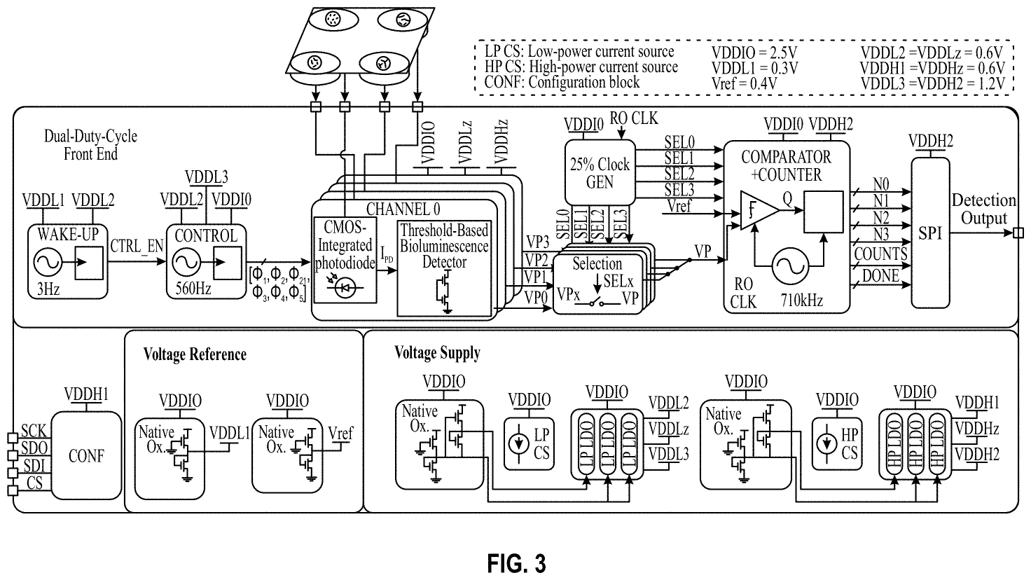

The bioluminescence detector shown in FIG. 3 performs luminescence detection from three independent bacterial biosensors via CMOS-integrated photodiodes. A fourth channel may be reserved as a reference to calibrate the background light and temperature and channel variations. The bioluminescence detector has three important features: (1) a CMOS-integrated two-by-two photodiode array to provide a scalable biomarker detection platform, (2) a threshold-detection-based bioluminescence processing circuit to achieve high sensitivity and resolution with low energy consumption, and (3) a sleep/wake activation through a dual-duty-cycling front end to lower the average power consumption. This application-specific integrated circuit (ASIC) has four parallel hardware channels consisting of a CMOS-integrated photodiode and a threshold-based bioluminescence encoder embedded in each channel. First, within each chamber, specialized bacterial biosensors will bind to their respective target biomarkers causing a light signal (e.g., bioluminescence) to be emitted, which is then converted to a photocurrent by the photodiode. Subsequently, this photocurrent is encoded into threshold-crossing time information, annotated as (tVp), which is processed by a shared time-to-digital converter to quantify the light level and output digital counts proportional to the signal strength, hence constructing a detected biomarker level. The time-to-digital converter is composed of a comparator and a counter sharing a ring oscillator. Additionally, since the four parallel channel outputs share the time-to-digital converter, time-interleaved switches, driven by a 25%-duty-cycle clock at 178 kHz, were used to select the active channel. The digital counts from the four hardware channels can be read out through an on-chip digital controller equipped with a Serial-to-Parallel (SPI) interface operating at 160 kHz and 1.2 V. The SPI communicates externally to an on-board microcontroller with a wireless transmitter (PIC12LF1840T39A, Microchip), which then relayed the counts wirelessly to a separate base station (CC1200, Texas Instruments) located 1-3 m away. This setup was used for the optical characterization and in vitro testing.

Duty cycling is used for reducing the average power consumption. During the first level of duty cycling, a global slow wake-up timer activates the ASIC for bioluminescence detection. The relevant biological events occur over a slow transient process, typically minutes to hours, thus the wakeup timer is designed to support a tunable frequency range of 2 to 6 Hz for activating the ASIC. When the CTRL_EN signal generated by the wake-up timer becomes high, a shared control block provides clock signals Φ1 to Φ5 generated by a gate-leakage-based oscillator operating at 560 Hz and a programmable counter. These specific clock cycles provide the mechanism for encoding the light signal into time information while enabling the second level of duty cycling. Furthermore, the ASIC includes 2- and 4-transistor voltage references, current references, and low-dropout regulators (LDOs) for supplying different voltage levels for the circuit blocks.

A. Custom Photodiode Design in Standard CMOS Technology

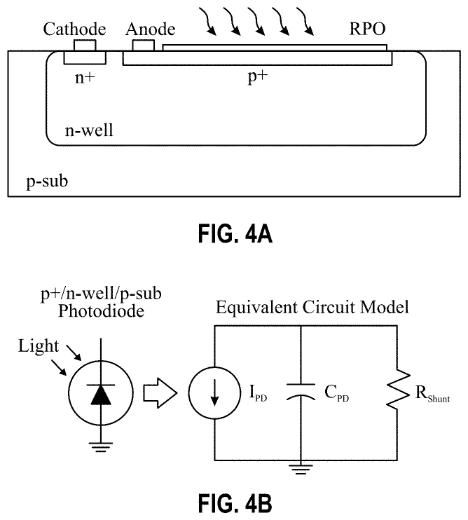

Three main diode types were available for design in standard CMOS processes: n+/p-sub, n-well/p-sub, and p+/nwell/p-sub. One important performance parameters of a CMOS photodetector are responsivity and dark current level. These parameters depend on the process technology, diode type, and diode geometry. The p+/n-well/p-sub diode was selected to implement photodiodes in the proposed architecture primarily due to its high responsivity. Each hardware channel converts the light signal emitted by biosensors to a photocurrent using a single p+/n-well/p-sub photodiode with a dimension of 1.24 mm×1.24 mm and a sensitive area of 0.9 mm². The array of photodiodes provided near simultaneous measurements from three independent biosensors and a reference channel, where each diode is placed at a corner of the chip. The cross-section of a p+/n-well/p-sub photodiode is illustrated in FIG. 4A. The metal contacts were placed only on the side of the active areas to increase the fill factor. An additional resist-protection-oxide (RPO) layer blocks the silicide diffusion, converting a semiconductor pn-junction diode into a photodiode. A CMOS-integrated photodiode can be represented by an equivalent model consisting of a DC current source IPD in parallel with a shunt resistor (Rshunt) and a junction capacitor (CPD) as shown in FIG. 4B. The shunt resistance is measured by sweeping a small range of negative to positive voltages, e.g., VPD=−10 mV to 10 mV, across the photodiode and reading out the diode current to back-calculate the resistance. Hence, Rshunt is defined as the slope of the current-voltage curve when the bias voltage across the photodiode is close to the origin (VPD=0). The shunt resistance of photodiodes on this chip was measured as ≈26 GΩ using a standalone chip discussed in Section IV-A.

The photodiode junction capacitance CPD is proportional to the diffusion area and inversely proportional to the bias voltage across the photodiode (VPD). In the equivalent model, the CPD has the largest value when the applied bias across the photodiode is 0 V. Responsivity is defined as the sensitivity times the junction capacitance, where sensitivity of a photodiode is defined as the output voltage per unit time divided by the optical input power in V/s·W. The p+/n-well/p-sub type photodiode has two junction capacitances in parallel, (1) p+ to n-well and (2) n-well to p-sub, thus, introducing a greater junction capacitance than the other types of pn-junction diodes in the CMOS process. All three types of diodes surveyed in previous work had the same sensitivity at large diode area and 490 nm wavelength, therefore the one with the highest capacitance is expected to have the highest responsivity embedded within it. Hence, higher responsivity is expected by using the p+/n-well/p-sub photodiode.

To obtain the highest responsivity and lowest dark current, a zero-bias voltage across the photodiode was maintained. Within this region, before the photodiode entered the forward bias condition, the overall photodiode current IPD was approximately equal to the photocurrent generated from an incoming optical signal, e.g., the net light emitted by the bacterial biosensors. Therefore, the bias voltages across the four photodiodes was reset to zero (0 V) for every measurement.

### III. Circuit Implementation Details

Details regarding the circuit implementation are discussed belong, along with some design decisions to achieve scalability, low power, high sensitivity, and resolution for multiplexed bioluminescence measurements via a miniaturized ingestible capsule.

A. Threshold-Based Bioluminescence Detector

Previous fully-integrated bioluminescence detectors use a CMOS-integrated photodiode followed by a continuous-time operational-transconductance amplifier (OTA)-based integrator with high gain and low noise. However, the continuous time OTA-based detectors consume mW-level power, e.g., 3 mW. These fully integrated solutions require a large battery limiting the miniaturization of an ingestible capsule. In contrast, the luminescence detector in other works consumes 26 nW of active power, employing a two-by-two array of commercial phototransistors. However, the use of off-chip commercial phototransistors limits the scalability of the multi-diagnostic system, as this solution is packaged in a large ingestible capsule (3 cm by 1 cm), which adversely impacts human safety and ease of use.

As described in this example and elsewhere herein, a threshold-based bioluminescence detector was designed by integrating CMOS photodiodes to address the miniaturization challenges describe above, while achieving high-sensitivity and high-resolution readout under a tight energy budget. The proposed readout system replaced the power-hungry continuous-time OTA-based integrator in previously existing fully-integrated bioluminescence detectors and/pr previously existing fluorescence detectors with a discrete-time front end for low power consumption without sacrificing the integration level and precision. The discrete time front end consists of a custom dynamic time-threshold-based detection circuit and discharging current source similar to the circuit-level techniques used in zero-crossing-based analog-to-digital converters (ADCs).

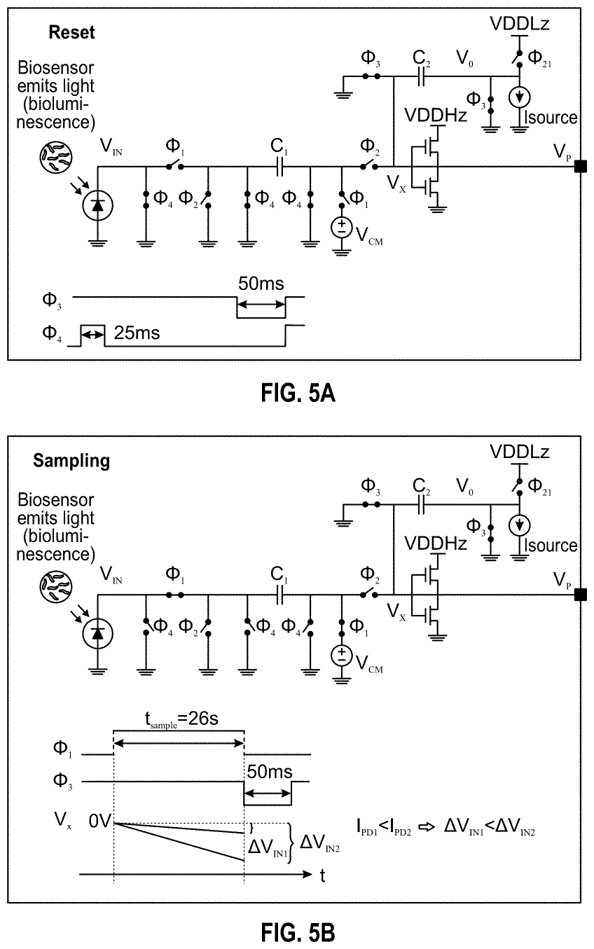

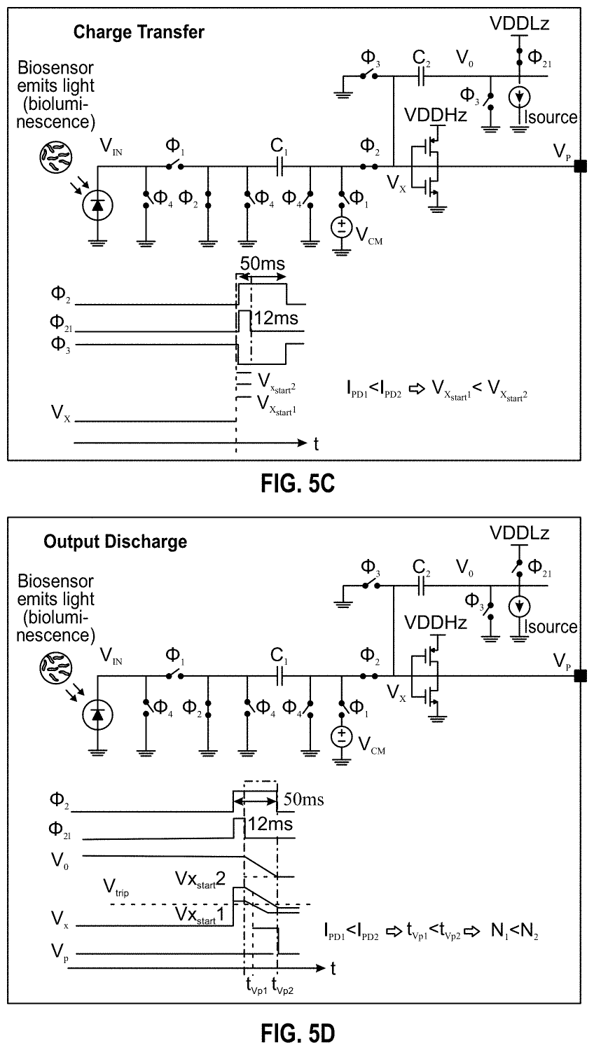

FIG. 5 shows the detailed operation of the threshold-based bioluminescence detector. At the beginning of each measurement cycle, the junction capacitor of each photodiode CPD and capacitors C1 and C2 are discharged through switches controlled by clock signals Φ3 and Φ4 as shown in FIG. 5A.

In the sampling phase illustrated in FIG. 5B, Φ1 is designed to be closed for a period (tsample) of 16-26 seconds, which can be programmed by the control block. The photodiode current IPD, composed of desired photocurrent generated from bioluminescence (IPH) and unwanted dark current (ID), is integrated on the diode junction capacitor (CPD) over this sampling period (tsample). Negative bias voltage VIN across the photodiode junction capacitor CPD is sampled on the top plate of the capacitor C1=20 pF, whereas the bottom plate samples VCM, which is generated via a 2-transistor voltage reference and set to 0.3 V and it is equal to half VDDHz of the threshold-based detection circuit. This builds up a total charge of Qsample=C1·(VCM+ΔVIN). Since VIN is reset to zero by discharging CPD at the beginning of every measurement, ΔVIN is given by:

ΔVIN=0−(−VIN)=VIN=IPD*tsample/CPD (1)

The dark current is minimized when applied bias across the photodiode is zero to ensure a linearly proportional relationship between the photodiode current (IPD) and the optical power of bioluminescence produced by the bacterial cells. In these designs, the bias voltage was reset across the photodiode at the beginning of each measurement cycle, and set the sampling period duration (tsample) such that the maximum bias voltage across the photodiode does not exceed 20 mV.

During the charge-transfer phase shown in FIG. 5C, Φ2I was active for a short period, ≈3 ms to 12 ms, concurrently with Φ2, ≈12 ms to 50 ms, to reset VO node to the V DDLz voltage of the threshold-based detector block, VDDLz=VDDHz=0.6 V. The active period of Φ2 is configured through the programmable counter, discussed in Section III-B, below. The active period of Φ2I is equal to ¼ of the active period of Φ2. The expected maximum photocurrent, SPI and transmitter frequency, and the communication delay between on-chip and off-chip components set the optimum value of Φ2 active time. The charge built up during the sampling phase is transferred to C2=5 pF, setting VX node to a voltage level that is linearly proportional to bias voltage across the photodiode VIN during the Φ2I active period:

Qtransfer=C1·VX+C2·(VX−VDDLz) (2)

Qsample=Qtransfer (3)

VXstart=[(VCM+ΔVIN)·C1+VDDLz·C2]/(C1+C2) (4)

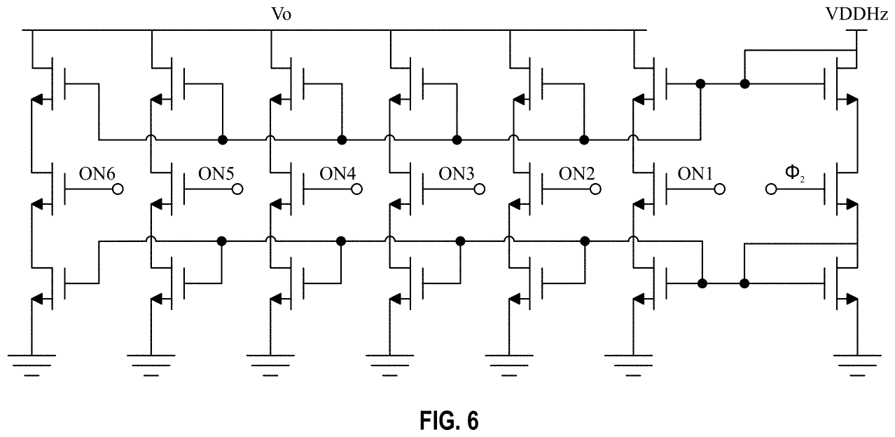

The threshold-based bioluminescence detector enters an output discharge phase once Φ2I is inactive as shown in FIG. 5D. VX discharges by a programmable 6-branch pseudo current source (Isource) enabled by Φ2 as shown in FIG. 6 at the rate of Isource/C1. Each parallel hardware channel contains an independent current source. The current level, 160 to 960 pA at 37° C., is configured through ON1-ON6 static digital bits set by the digital configuration (CONF) block based on system requirements detailed in Section IV below biosensors yields a larger input bias voltage across the photodiode (if IPD1<IPD2, then ΔVIN1<ΔVIN2), resulting in a higher starting voltage at the VX node (VXstart1<VXstart2). Given different initial voltages of VX, the crossing times (tVp) of the inverters' threshold (Vtrip) will linearly vary with IPD, and, hence, VIN. A higher VXstart voltage requires a longer time to cross the inverters' threshold Vtrip, which is set at 300 mV for this work. The threshold-crossing time (tVp) can be back-calculated from count values, N, reported by the SPI at 160 kHz. The tVp depends on the discharging rate (Isource/C1), initial voltages of VX, and the inverters' threshold voltage (Vtrip). The resolution of the threshold-based bioluminescence detector is defined as:

Resolution=ΔIPD/ΔtVP=(ΔIPD)Isource/C1*ΔVX=(ΔIPD)Isource/[C1Δ(VXstart−Vtrip)]=(ΔIPD)Isource/[C1*ΔVXstart] (5)

B. Wake-Up Timer and Control Clock Generator

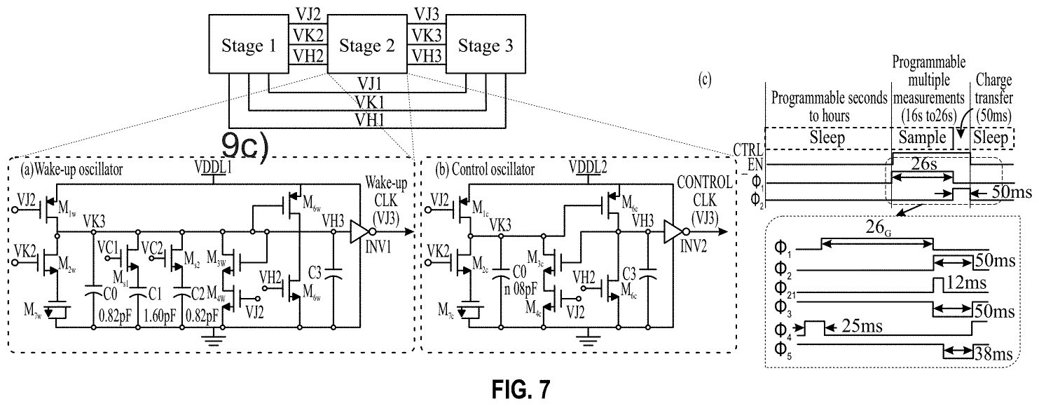

In this system, gate-leakage-based three-phase oscillators achieving low energy per cycle were chosen for generating the slow wake-up timer and low-frequency control clocks for the detector. As shown in FIG. 7, a gate leakage transistor M7w provides a small reference current (60 fA) to discharge the capacitor network (C0,C1,C2) until the threshold voltage of M5w is reached. A strong turn-on of M5w flips the voltage across capacitor C3, hence inverter's input (INV1 in FIG. 7), with the help of feedback transistor M3w. Each of these dynamic delay stages are connected in series to form a low-energy three-stage relaxation oscillator as shown in FIG. 7. A low-power 2-transistor voltage reference generated a stable voltage reference of VDDL1=0.3 V from the main supply to support the wake-up oscillator operation in both active and sleep modes. The wake-up oscillator was tunable through configuration bits VC1 and VC2 to obtain a slow clock with a nominal frequency of 3 Hz and achieved a measured temperature coefficient of 1.3% per degree with a simulated energy consumption of 1.63 pJ/cycle. A programmable digital wake-up counter using the output from the wake-up oscillator generated an enable signal CTRL_EN to activate the remaining analog front-end blocks, which formed the first-level of duty cycling as shown in FIG. 7. The second programmable counter used a 560 Hz clock generated by a gate-leakage-based three-phase oscillator, shown in FIG. 7, with no tuning capacitors. This counter generated several switch-control signals, Φ1 to Φ5, shown in FIG. 7. Φ1 ranged from 16-26 seconds to avoid forward biasing of the photodiode. The control oscillator achieved a measured temperature coefficient of 10% per degree with a simulated energy consumption of 342 fJ/cycle. These gate-leakage-based oscillators offer significantly low energy consumption per cycle at the expense of degraded temperature stability performance. Since the application of interest was an ingestible capsule for disease diagnosis and monitoring, the temperature was stable at ≈37° C. for nominal body temperature. Given the low energy consumption of the gate-leakage-based oscillators, both wake-up and control oscillators were allowed to continuously run.

C. Time-to-Digital Converter

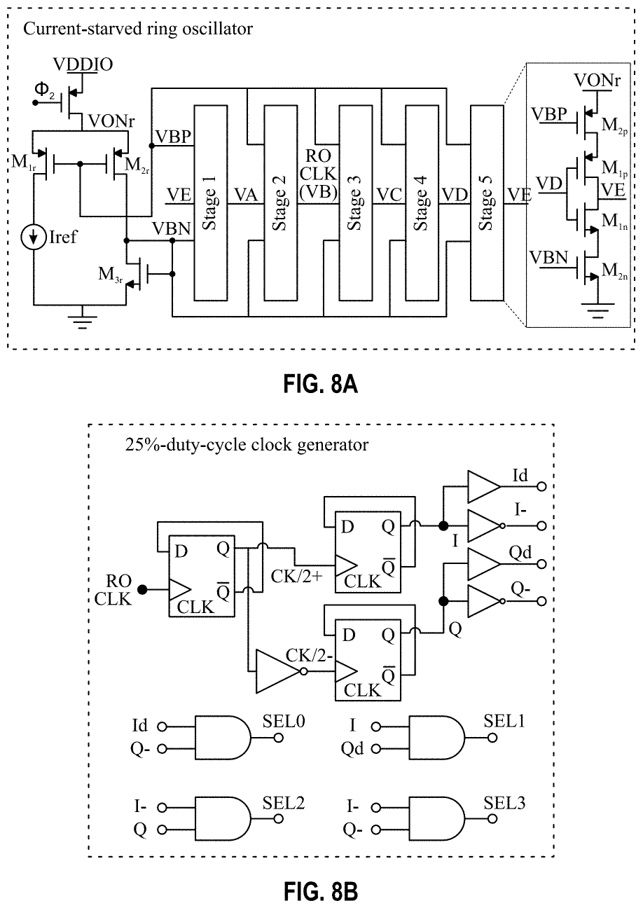

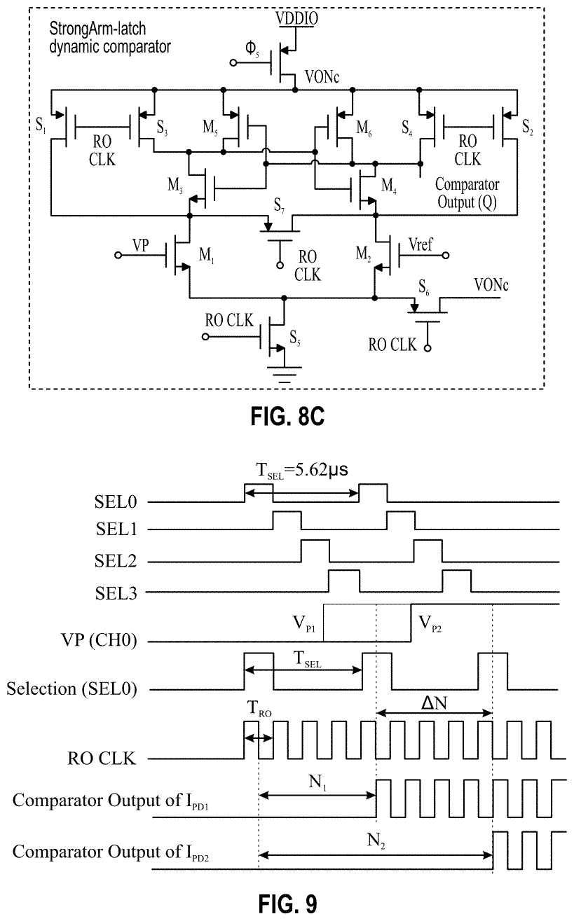

The output of each threshold-based bioluminescence detector channel was a step voltage (VP) from 0 V to VDDHz=0.6 V. An analog multiplexer was designed for time-interleaving of the four parallel channels. These time-interleaved switches were driven by a 25%-duty-cycle clock generator to select the active channel output VP for digital processing. The analog multiplexer output, VP signal, was fed to a StrongArm-latch comparator, shown in FIG. 8C, clocked by a current-starved ring oscillator with a measured frequency of 710 kHz. The VP voltage was compared to a voltage of Vref=0.4 V provided by a 2-transistor voltage reference. As shown in FIG. 8A, the current through M1p and M1n of five-stage inverter-based RO was constrained by the current mirror formed by M1r−M3r to M2p, and M2n. The current flowing through the inverter chain could be adjusted via a tunable Iref that was current mirrored from a high-power on-chip current source (HP CS) as discussed in Section III-D, below. The 25%-duty-cycle clock generator, shown in FIG. 8B, clocked by the current-starved RO, was composed of two-stage divide-by-2 circuits and buffers/inverters to obtain delayed clocks (Id and Qd) and inverted clocks (I- and Q-) at ¼ of the RO clock frequency. These signals, Id, I-, Qd, and Q-, were provided to AND gates for obtaining 25%-duty-cycle channel selection signals, SEL0-SEL3. As shown in FIG. 8C, initially when RO clock is low, both M1 and M2 were off. Once RO clock goes high, S1-S7 turn off, and transistors M1 and M2 are on, drawing a differential current in proportion to VP and Vref and discharging drain voltages on M1-M2. If VP is greater than V ref, the discharging on M1 happens quicker compared to M2 and crosses the threshold voltage of M3 earlier. M3 turns on, allowing discharging current to flow through M6 and creating a rising edge of the comparator output (Q). A synthesized digital counter, Q counter, takes RO clock, selection signals (SEL 0-SEL 3), and comparator output (Q) as inputs. The Q counter keeps track of the number of clock cycles until each channel observes its first rising edge of the comparator output. FIG. M8 illustrates the input and output signals of the time-to-digital converter for the example scenario. Given that photocurrent IPD1 is smaller than IPD2 for channel 0, the crossing time of the inverters' threshold (Vtrip) due to IPD1 was shorter than the crossing time of the threshold (Vtrip) due to IPD2. Consequently, the comparator output Q had its first rising edge earlier for IPD1, and, hence, the Q counter output a smaller N1 compared to N2. The threshold-crossing time of each channel and resolution based on the number of digital counts and the clock period of the ring oscillator can be defined as:

Resolution=ΔIPD/ΔtVP=ΔIPD/(ΔNi×TRO) (6)

where ΔIPD is the difference between photocurrents, e.g., IPD1 and IPD2, corresponding to different bioluminescence levels, ΔtVP is defined as the difference between the threshold-crossing times for two different photocurrent levels, ΔNi is the difference between N1 and N2 as shown in FIG. 9, and TRO is the period of the current-starved ring oscillator clock. The current-starved ring oscillator, the 25%-duty-cycle clock generator, the dynamic StrongArm-latch-based comparator, and the digital Q counter are activated only when Φ2 (12-50 ms) is high. This second level of the dual-duty-cycling scheme allows us to minimize the average power consumption of the higher-frequency circuits.

D. Power Management Unit

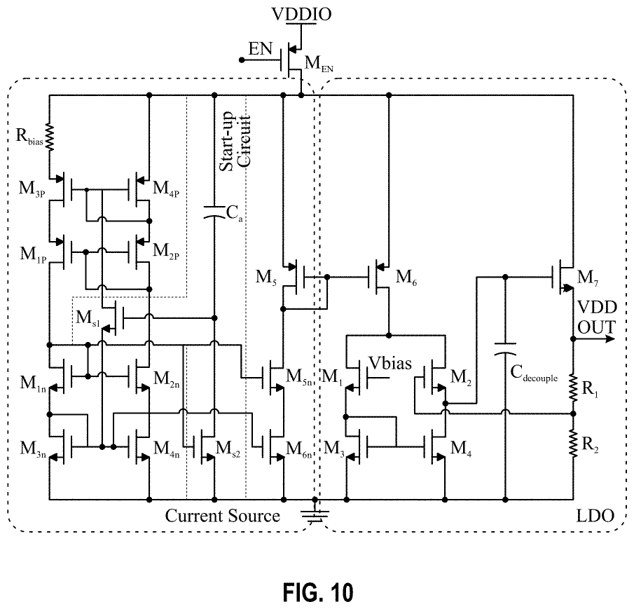

The power management unit consisting of two on-chip LDO/current reference pairs provided stable supply voltages at levels, 0.6 and 1.2 V, to different circuit blocks by drawing current off the main supply of the ingestible capsule, a coin cell battery (5.5 mAh) providing 2.5-3 V. The first pair, LP CS and LP LDO, were designed to operate at low average current levels (≈7 nA) for supplying the low-power oscillators, level shifters, and the reset of the VO node of the threshold-based bioluminescence detectors. The second pair, HP CS and HP LDO, were designed to operate at a higher average current level (≈0.6 μA) to support power-hungry digital controllers, e.g., SPI. The HP CS and HP LDO are activated when Φ5 was active low for 38 ms. Both HP and LP LDOs with their current sources had similar architectures as shown in FIG. 10. The proportional-to-absolute-temperature (PTAT) current reference with a startup circuit, formed by Cs, Ms1, and Ms2, leverages the bias resistor Rbias value to set the average current levels and provide a reference current for the LDO. The on-chip LDO consists of a power transistor (M7) and a single-stage differential error amplifier. The positive error amplifier input monitored the fraction of the LDO output set by the resistor ratio of R1 and R2, while the negative error amplifier input was connected to a stable bias voltage, Vbias, generated by a 4-transistor voltage reference.

### IV. Measurements

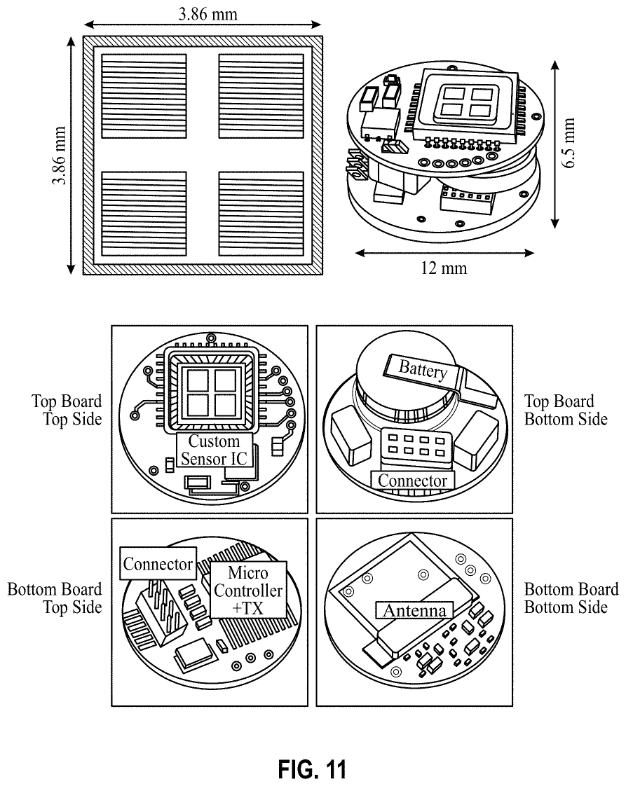

The chip was fabricated in 65 nm CMOS technology. FIG. 11 shows the chip micrograph as well as the printed circuit boards (PCB) hosting the ingestible multi-diagnostic capsule system in a mm-scale form factor. The upper side of the top PCB holds the quartz-lid QFN-packaged custom-designed CMOS multi-channel and time-multiplexed bioluminescence detector. A 6.8 mm×2.1 mm coin-cell battery (MS621FE, Seiko Instruments) provided the main supply voltage for all of the components on the PCB. The commercial wireless microcontroller and transmitter integrated circuit (IC) with its matching network that connects to an antenna (0915AT43A0026, Johanson Technology) are located on the bottom PCB. The microcontroller writes the configuration bits into the custom CMOS bioluminescence chip's digital controller block. Once the microcontroller detected an interrupt signal from the bioluminescence chip indicating threshold requests the digital counts corresponding to the threshold-crossing time of the bioluminescence detector from the on-chip SPI. The wireless transmitter IC communicates out of body to a commercial receiver (CC1200, Texas Instruments). Each wireless packet is 232-bits long with a preamble/header and was transmitted at 40 kbps with +10 dBm power, resulting in a wireless transmission for 5.8 ms at 915 MHz using a frequency-shift-keying (FSK) modulation. In the following subsections, the optical measurements of the ingestible capsule chip are shown. Second, the in-vitro performance of the bio-engineered system was shown with the genetically engineered bacterial sensors. This section is concluded with the performance comparison against the state-of-the-art implementations.

A. Optical Measurements

Preliminary characterization of the genetically engineered bacterial sensors was performed using a standalone p+/n-well/p-sub photodiode chip fabricated in 65 nm CMOS technology without additional readout circuits. This standalone chip included a photodiode with the a similar size and physical structure as the photodiodes of the custom CMOS bioluminescence detector described above. The standalone chip was connected to a sub-femtoamp source measurement instrument (K6430, Keithley Instruments) with 0 V across the photodiode. To measure the maximum expected light signal from the biosensors, 2 μL of cells contained in an open plastic well with an optically clear thin film bottom (0.1 mm thick), were placed onto the photodiode. The characterization shows that the genetically engineered biosensors yield very low-level optical signals with a maximum of 270 fA. As shown in Eq. 5 above, the resolution value is linearly proportional to the discharging current Isource. Therefore, only one branch of the discharging current source was enabled to ensure the best resolution, which represents a lower resolution value, for the ingestible capsule.

The wireless ingestible capsule, the standalone photodiode IC, and an optical meter (PM100D, ThorLabs) were placed inside a metal box, to avoid electromagnetic interference on the long wires used for this special calibration mode. In addition, the box was covered with a blackout cloth to avoid ambient light from the environment during the optical characterization. The temperature was set to 37° C. by placing the metal box inside a temperature-controlled incubator, similar to all biological measurements. Five different voltage levels (0 V, 2.1 V, 2.14 V, 2.165 V, 2.185 V) were supplied across a teal LED (λ=520 nm) placed 30 cm opposite to both ICs and the optical meter. Different supply voltage levels across the LED yielded different optical power levels, simulating different luminescence levels. The ingestible capsule continuously measured and transmitted a packet wirelessly every 26 s (i.e., the sampling period was set to 26 s) to the receiver. Total 100 readout packets were transmitted at each LED bias voltage level. At the same time, both the optical meter and the standalone photodiode reported corresponding optical power level and photocurrent level for this given LED bias voltage. The optical meter with a sensor size of 0.7088 cm² was placed equidistant, 1.5 cm, from the ingestible capsule and the standalone photodiode IC. The sub-femtoamp source meter reported the real-time photocurrent outputted from the standalone photodiode IC. The ingestible capsule IC's second channel (CH 2) was covered by a black masking tape, serving as a reference channel for calibration. When the supply voltage across the LED was 0 V, i.e., no light in the metal box, the ingestible capsule readout from each channel was utilized for one-time calibration of the digital-count offset per channel.

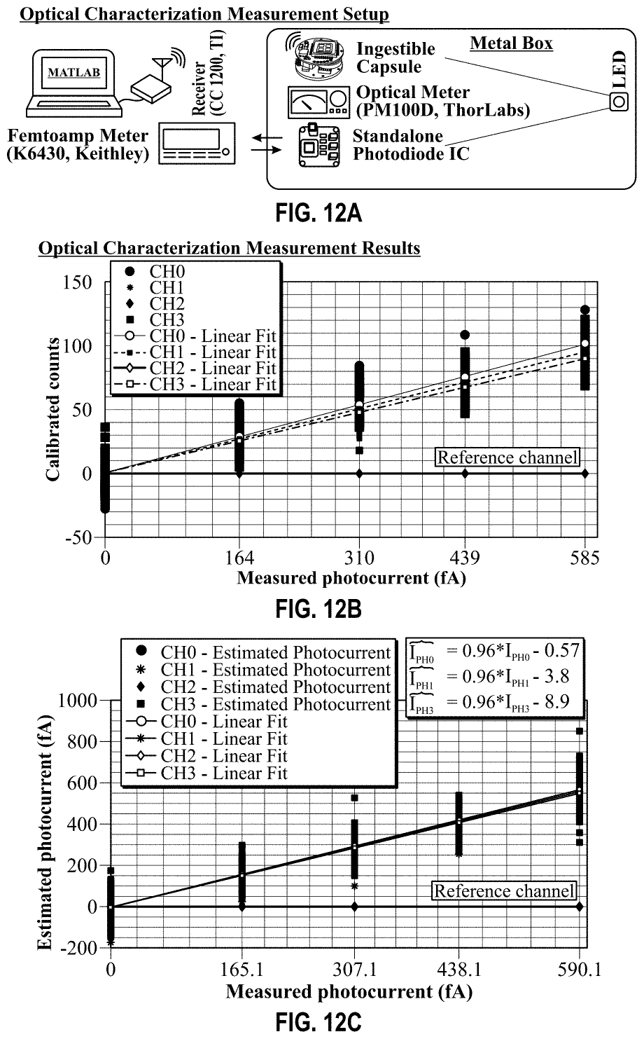

To characterize and obtain the measured MDS and resolution, training and test data was collected. FIG. 12A shows a schematic diagram of the setup for this data collection. First, the training optical-characterization dataset was measured by sweeping the five LED bias voltages in a light and temperature-controlled environment. The resolution of each channel was obtained via measuring the quantities as defined in Eq. 6 above. The ΔNi is the difference between the luminescence detector IC output counts for two LED bias voltages, and the ΔIPD is the difference between photocurrent values reported from the standalone photodiode IC for the same two bias voltage settings. As shown in FIG. 12B, the individual channel resolution is calculated as the inverse of the slope of the 1st-order polynomial curve fitting from the measured 500 calibrated digital counts of the luminescence detector IC (y-axis) versus measured photocurrents outputted by the standalone photodiode IC (x-axis). The measured resolution values range from 4.11 to 4.62 fA/μs. The MDS is calculated using the average resolution of each channel times 1σ standard deviation of the calibrated digital counts at a given LED bias voltage. The worst-case MDS among the four parallel channels is 71 fA with an average MDS of 59 fA. Second, a test optical-characterization dataset was measured the next day using the same experimental setting as the training dataset and used to verify the accuracy of the photocurrent estimation from the training optical characterization. The x-axis data of FIG. 12C (IPH) is provided by the testing data measured from the standalone photodiode IC, and the y-axis data was provided by the estimated photocurrent (dIPH), which was calculated by multiplying the measured digital counts from the testing dataset with the resolutions obtained from the training dataset. Note that the training data provides the nondimensionalization of the number of digital counts from the capsule to report consistent photocurrent levels across multiple experiments. A moving window averaging could be applied later to the calibrated counts due to long biological time constant and this will reduce the noise. Equations denoted in FIG. 12C shows the linear fit curves of dIPH for individual sensing channels. Given that the slopes of the linear-fit curves for the sensing channels are close to 1 with less than 5% offsets, these optical measurements validated a one-time optical calibration procedure, and, hence, the substitution of resolutions from the optical calibration is valid for the in-vitro biological measurements.

B. In-Vitro Biological Measurements

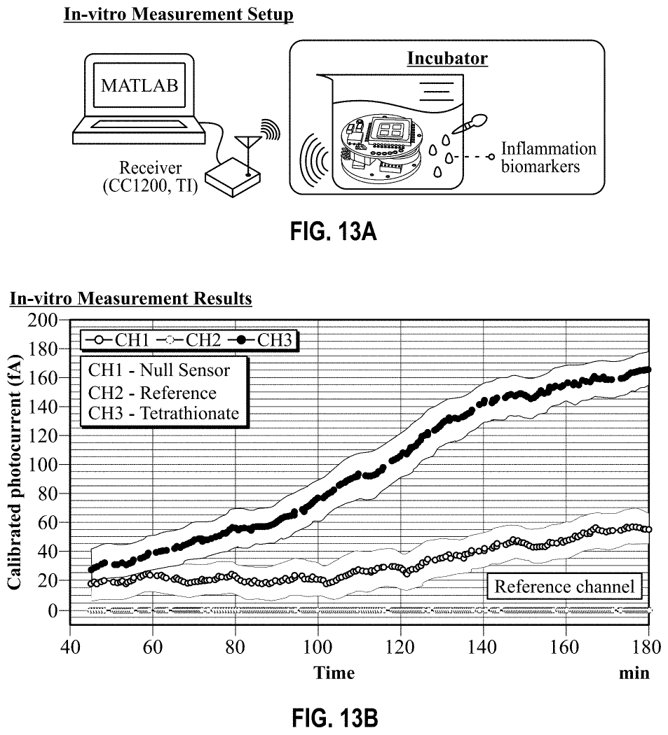

After completing the offset calibration and optical characterization of the bioluminescence detector IC, in-vitro measurements of the wireless ingestible capsule interfacing with the genetically engineered bacterial sensors were conducted. FIG. 13A shows a schematic illustration of a setup for this characterization. The bioluminescence detector chip was hermetically sealed under a quartz lid and was enclosed by bacterial chambers allowing maximum light transmission to the CMOS-integrated photodiode array. For the in-vitro measurements, the wireless ingestible capsule was coated with 2 μm of Parylene C to act as a moisture barrier. Parylene C coating was performed using Specialty Coating System Labcoter 2 (PDS 2010) following the same protocol described in previous work. The custom-designed 3D-printed casing encapsulated the coated capsule. Finally, the casing was sealed with Elite Double 22 (Zhermack Dental) to further reduce the chance of leakage.

For the in-vitro experiments, overnight cultures were diluted 1:10 in LB (Luria-Bertani) liquid medium, then subcultured for 20 minutes, and lastly concentrated 100× by centrifugation. A 1-1.5 μL of the concentrated bacterial cells were added to the bacterial chambers that are placed on top of Channel 3 (CH3). A 1-1.5 μL Wild-type E. coli Nissle 1917 processed the same way as the bacterial cells, which served as a null sensor with no bioluminescence production, was added to Channel 1's (CH1) bacterial chamber. Channel 2 (CH2) also served as an additional reference channel to calibrate offsets due to channel and temperature variations. Before initiating the in-vitro measurements, both LB media and the capsule chip were pre-warmed to 37° C. in the incubator for at least 10 minutes. The ingestible capsule and LB media were first placed into a beaker, followed by adding an intestinal inflammation biomarker (100 mM tetrathionate). At this point, we started the measurements. The capsule wirelessly transmitted data to a receiver (CC1200, Texas Instruments) over the duration of the experiment for three hours. After three hours of exposure to tetrathionate, CH3 detected the bioluminescence illuminated from the exposed tetrathionate sensor cells and reported a maximum 180 fA photocurrent, while CH1 provided a negative channel set by the system noise, as shown in FIG. 13B. The shaded error bars denote the 95% confidence interval for samples over a 45-minute sliding window.

C. Performance Summary and Comparison

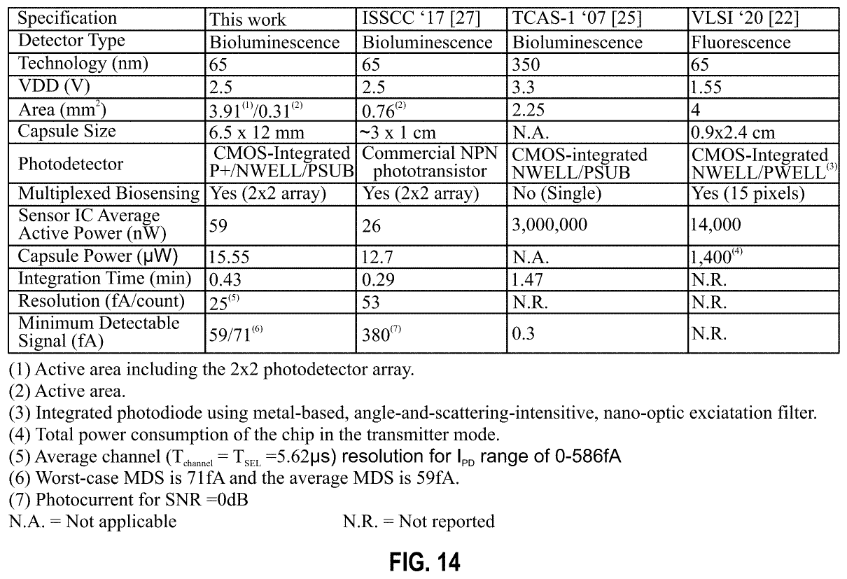

FIG. 14 summarizes the presented system performance and compares it against the state-of-the-art demonstrations. The results of the multi-channel and time-multiplexed bioluminescence detector IC consisting of a two-by-two array of CMOS-integrated photodiodes demonstrates 5.4× lower MDS and 2.1× higher resolution compared to the first state-of-the-art solution at body temperature (37° C.). Further, this chip consumed 59 nW of average active power while providing a multiplexed measurement capability, 50, 847× lower average active power compared to the second state-of-the-art solution. Finally, a significantly smaller form factor (6.5 mm by 12 mm ingestible capsule) was demonstrated, relative to the state-of-the-art cm-scale wireless ingestible capsules.

### V. Conclusion

The threshold-based bioluminescence detector IC exhibits a dual-duty-cycling front end with a two-by-two CMOS-integrated photodiode array achieving high sensitivity and high resolution while consuming 59 nW of active power. This example demonstrated the first molecular ingestible capsule at mm scale using the integrated CMOS threshold-based bioluminescence detector for simultaneous detection of different labile biomarkers. This system of this example provided a scalable detection platform agnostic to the biomarker of interest, which could serve as a minimally invasive tool for disease diagnosis and monitoring in the GI tract.

### Example 2

The following example describes the design and fabrication of several biosensors configured to detect one or more biomarkers.

The ability to diagnose and monitor inflammatory GI disorders would be transformed if one could profile labile, oxidation-related biomarkers and their responses to dietary change and therapies in situ. Many microbiome-related conditions, notably inflammatory bowel disease (IBD), are associated with chronic intestinal inflammation resulting from dysregulated immune homeostasis, specifically, increased oxidation. Malnutrition, antibiotic resistance, antibiotic dysbiosis, neurodegenerative diseases, and mitochondrial genetic disorders are also associated with redox imbalance in the GI tract, and poor responses to chemotherapy and vaccines, as well as aging, may also be underpinned by oxidative stress.

While the etiology of IBD is not well defined, bacterial infections and antibiotics may substantially increase concentrations of oxidants, such as reactive oxygen and reactive nitrogen species (ROS/RNS). These molecules may be labile, which may make it difficult to detect their presence or accurately measure their concentration in the body. While there have been some reports of devices that sense labile molecules in the GI tract (e.g. oxidizing gases, volatile organic compounds), they are limited to off-the-shelf sensors that use non-specific metal-oxide sensing elements. Thus, the current standard of clinical care is limited with respect to the capacity to provide evaluation of the chemical environment underlying the metabolic pathways of both the human host and its resident microbes. Developing non-invasive technologies that can continuously monitor the GI environment in situ would both expand the understanding of what causes inflammation and improve the effectiveness of therapies. Furthermore, diagnosing multi-faceted diseases such as IBD, in which biomarker levels vary greatly among patients, would greatly benefit from the simultaneous detection of a panel of oxidation-related biomarkers (e.g. nitric oxide [NO], ROS, thiosulfate [TS] and tetrathionate [TT]).

Current methods of diagnosing gastrointestinal (GI) inflammation include (i) endoscopy, which is invasive and should only be performed with limited frequency, and (ii) stool analyses, which may not accurately reflect intestinal conditions due to differential growth of certain species, ambient oxidation, and loss of labile disease-mediating molecules. Culture enrichment (e.g., as may be done for analyzing low-abundance microorganisms in stool samples with “omics” techniques) may also distort the initial bacterial ratio. Because the fecal microbiota only partially represents the autochthonous microbiota in direct contact with the intestinal mucosa, a biopsy may be required for a more complete analysis.

Electronic devices that continuously collect, process, and wirelessly transmit information can also be used to analyze the GI tract. However, capsule endoscopy cameras currently approved by the US Food and Drug Administration (FDA) may not directly measure the molecular mediators of disease, such as ROS/RNS. Other ingestible, ultra-low power electronic devices currently under development can be used to visually evaluate the GI tract and measure gas concentrations, temperature, and pH levels but require functionalization with fragile transducers to convert biochemical information into electronic signals, which limits specificity and robustness.

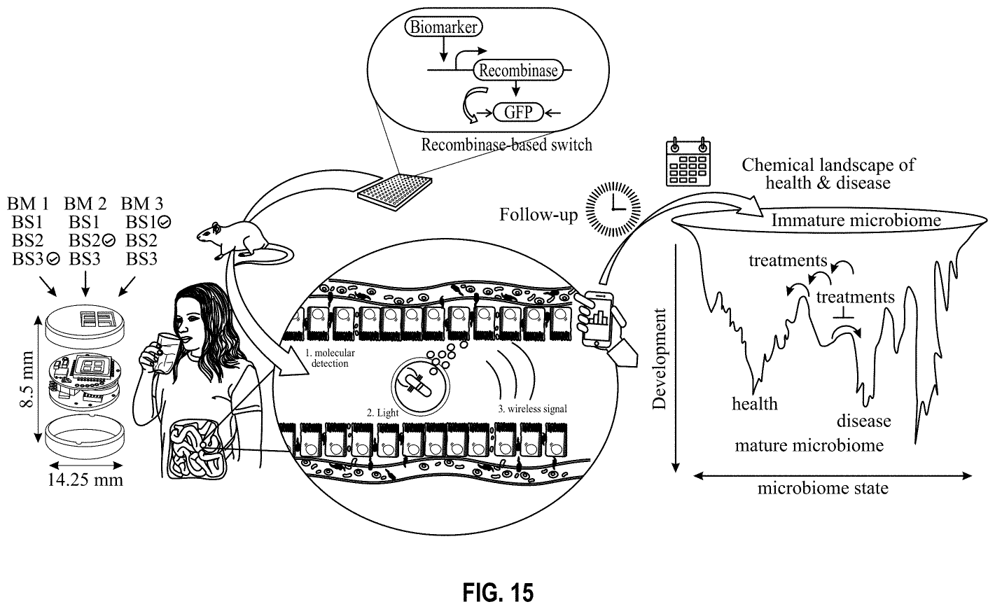

To overcome these challenges and to leverage the promise of transient biomarker-panels, natural protein-based sensing elements for NO, hydrogen peroxide (H₂O₂), TT, and TS were combined with genetically encoded memory circuits, incorporated them into probiotic bacteria, and validated their function in a rodent and porcine model of inflammation. These bacterial sensors were then integrated with a custom-designed integrated photodiode array and readout chip. The integrated systems has a volume below 1.4 cm³ and pill form factor conforming with a safe ingestible size for non-deformable dosage forms. Previous prototypes were validated for the detection of blood in vivo and was considerably larger (>9 cm³) than any known safe ingestibles. In addition, the miniaturized wireless bio-electronic device can safely process data with low-power consumption and transmit it to a portable device such as a smartphone. This multi-diagnostic device, in the form of a pill, has been tested in pigs, demonstrating that a human-scale diagnostic device can be built to detect transient mediators of GI inflammatory diseases (FIG. 15).

### Results

### Design of Biosensing Genetic Circuits

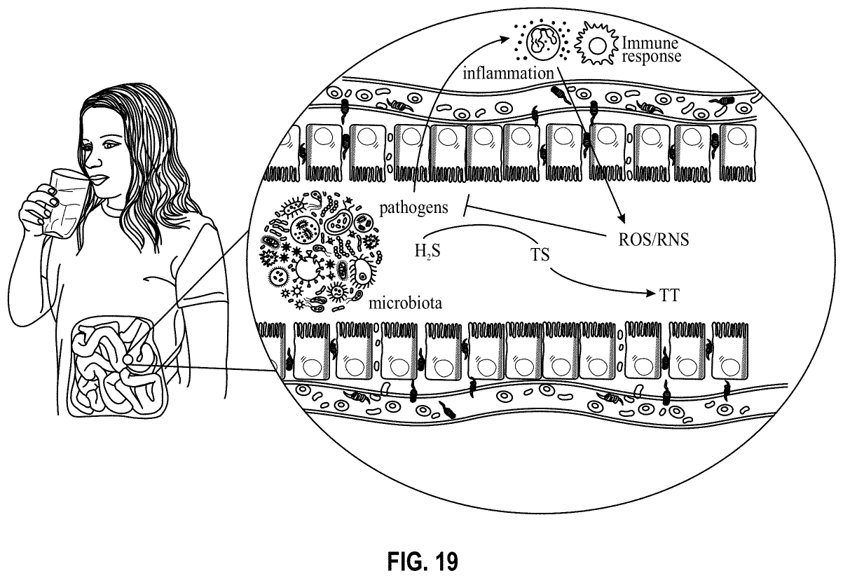

Intestinal bacteria may have natural sensors that continuously detect specific molecules in the gut. Considering the potential immunogenic response to diagnostic microbes, probiotic Escherichia coli Nissle 1917 was chosen as a chassis due to its resilience in the GI tract and excellent safety profile for long-term use and engineered it to detect the labile IBD-mediating molecules NO, H₂O₂, TS, and TT (FIG. 19).

Memory circuits were first created that could record NO exposure of bacterial biosensors as they traveled through the GI tract and then report any exposure when recovered from feces. Several bacterial NO sensors control the expression of NO reductases, which detoxifies NO inside the gut. The sensor was chosen NorR because it differentiates NO from other reactive nitrogen species (NOx) that are abundant in the gut environment. NorR activates transcription from the norV promoter (PnorV).

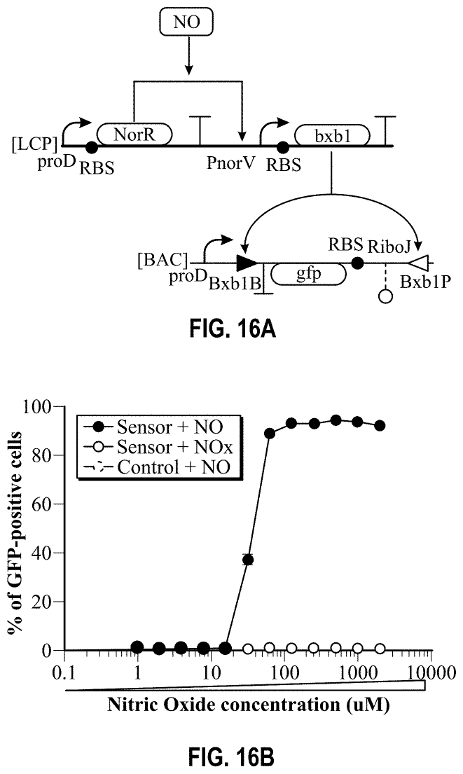

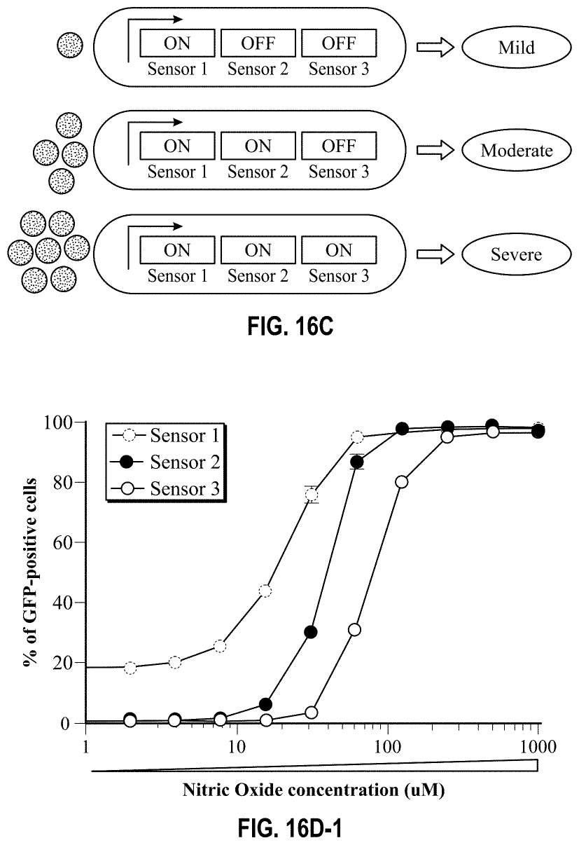

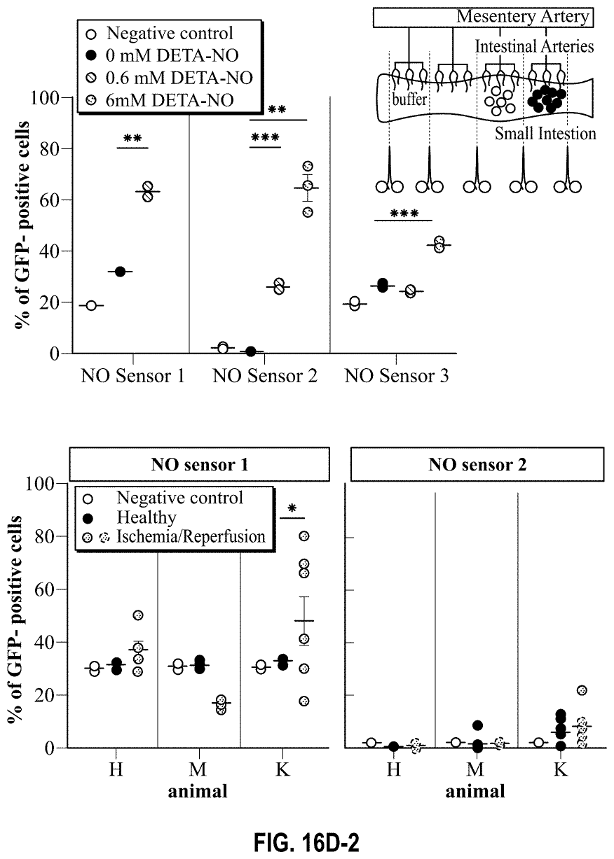

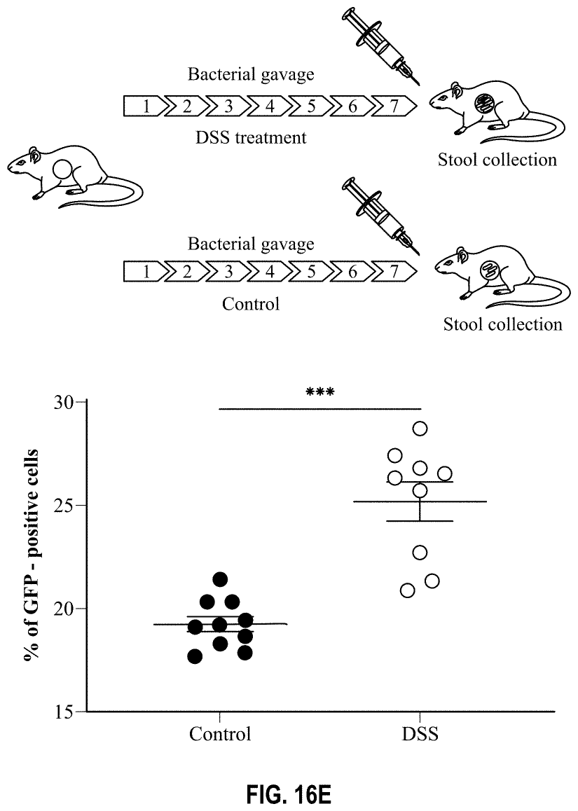

Recombinases recognize specific DNA sequences and can invert them, leaving long-lasting changes in DNA. To create memory circuits to report NO exposure, a bacterial NO biosensor was combined with a DNA recombinase core circuit (FIG. 16A). Once an exposure is recorded, the information may be stored in the DNA of the bacteria and passed from generation to generation. The information can then be retrieved by measuring green fluorescent protein (GFP) expression.

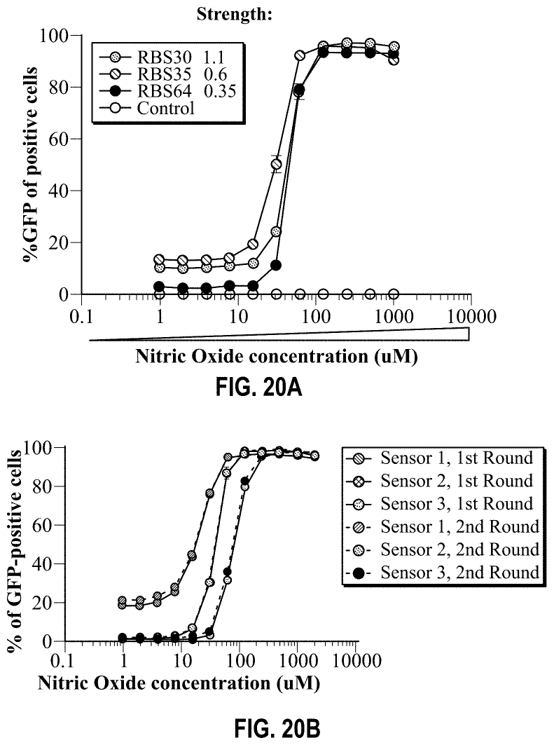

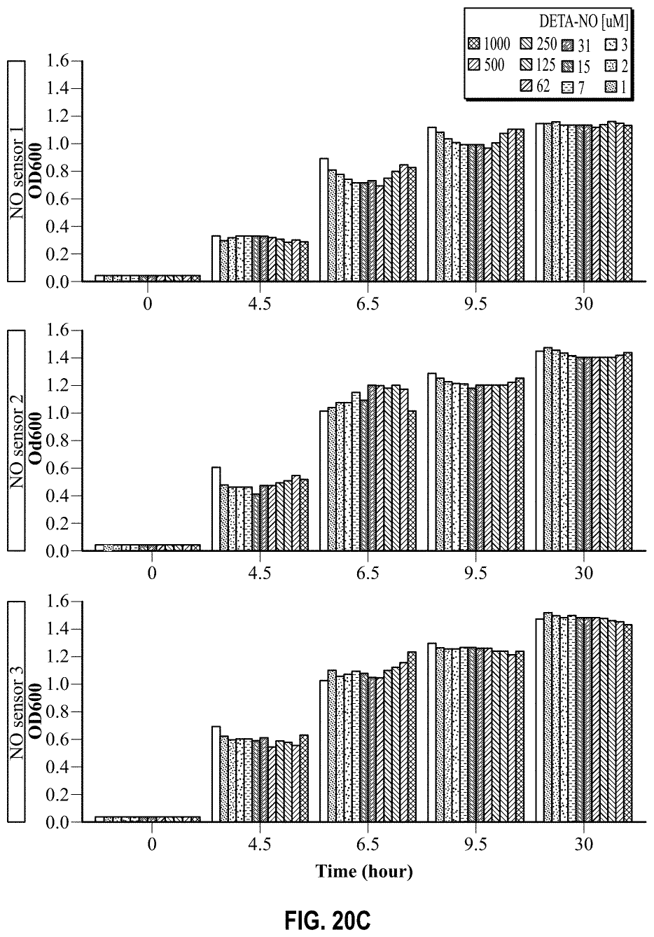

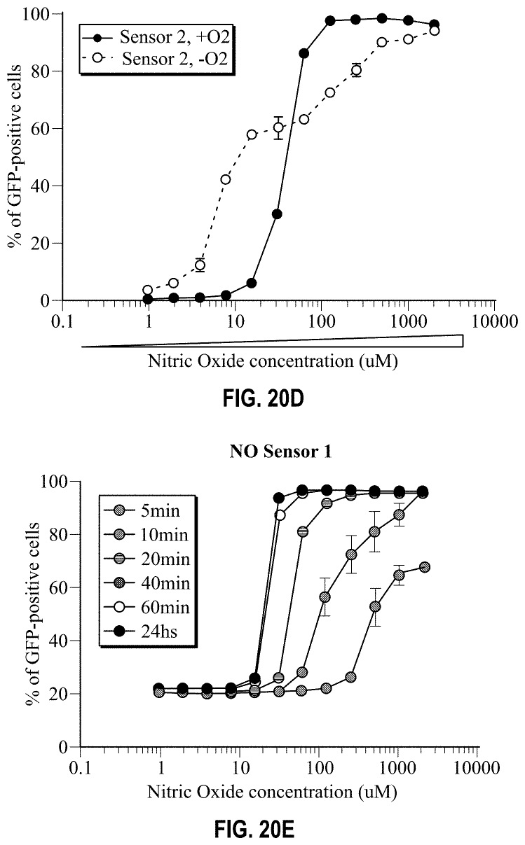

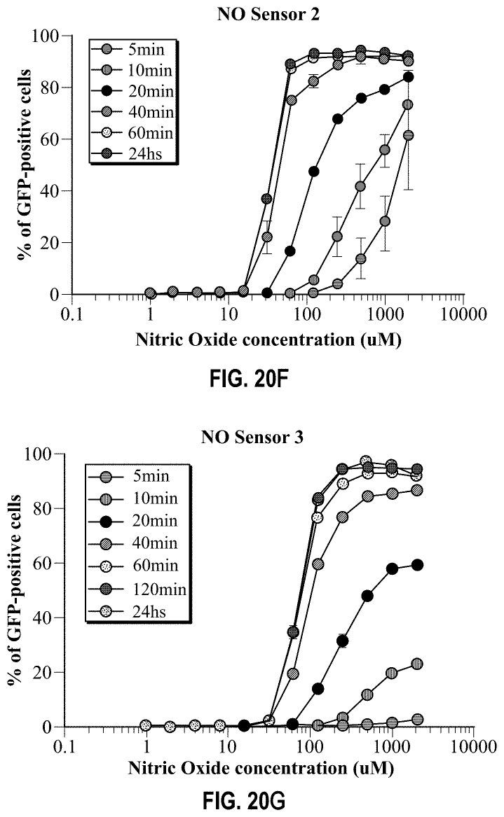

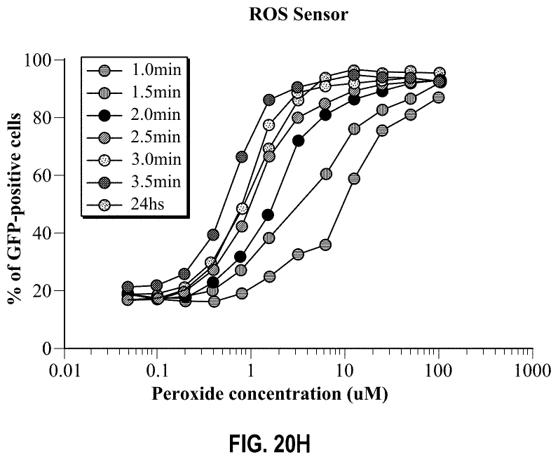

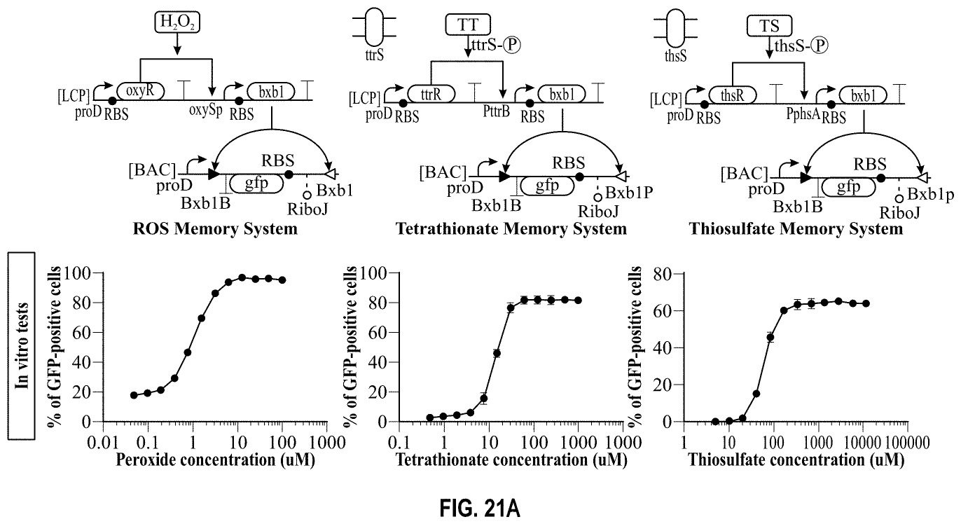

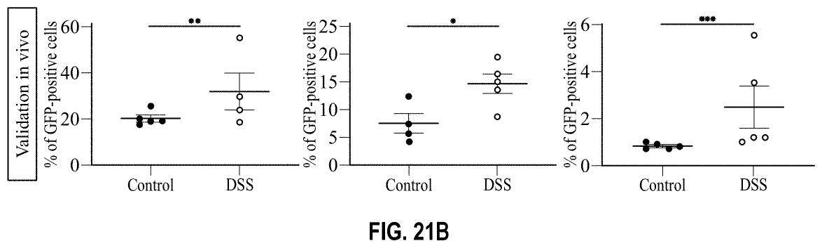

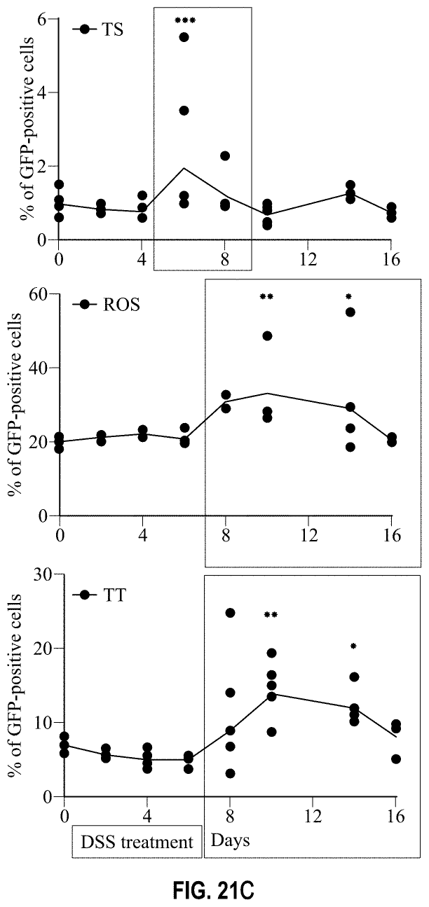

Using flow cytometry, GFP expression was measured and compared it to the concentration of DETA/NO (diethylenetriamine/nitric oxide adduct). The percentage of cells that were GFP ‘ON’ (% GFP−positive cells) was calculated at each concentration of NO. After multiple rounds of devices to improve the signal-to-noise ratio (SNR) of the genetic circuits and controls (FIG. 20A-20C), the NO recombinase-based memory system responded to a concentration threshold of 30 μM diethylenetriamine/nitric oxide (DETA/NO) and was not influenced by NOx compounds present in the diet, showing high specificity for NO (FIG. 16A). The performance of the NO sensor was tested under anaerobic conditions (FIG. 20D) and over time (FIGS. 20E-20H). Similarly, recombinase-based memory circuits were built for ROS, TS, and TT detection (FIGS. 21A-C).

A disease stage detector (DSD) was also created based on the concentration of NO detected, in which each stage could indicate a different level of inflammation—e.g., mild, moderate and severe (FIG. 16B). The incorporated recombinase-based switch was also used to discretize the biomarker input levels and create digital memories in the cells. Tuning the sensitivity of the NO biosensor resulted in different activation thresholds, so that physiologically relevant concentrations of the biomarker (e.g., low, medium, or high) could be measured in animal models. The sensors were developed for in vivo validation in mice measured GFP expression as a readout of the memory system activation; the sensors were activated within minutes (FIGS. 20E-20H).

### Disease Detection in Animal Models of IBD Via Bacterial Sensors

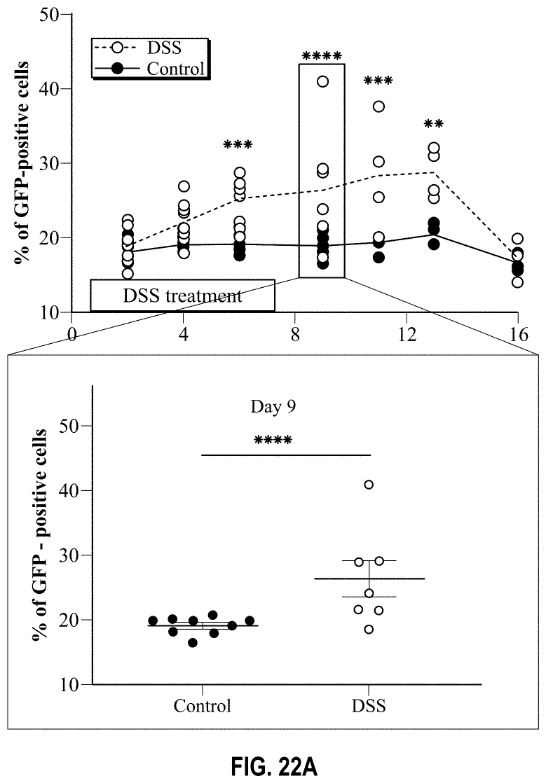

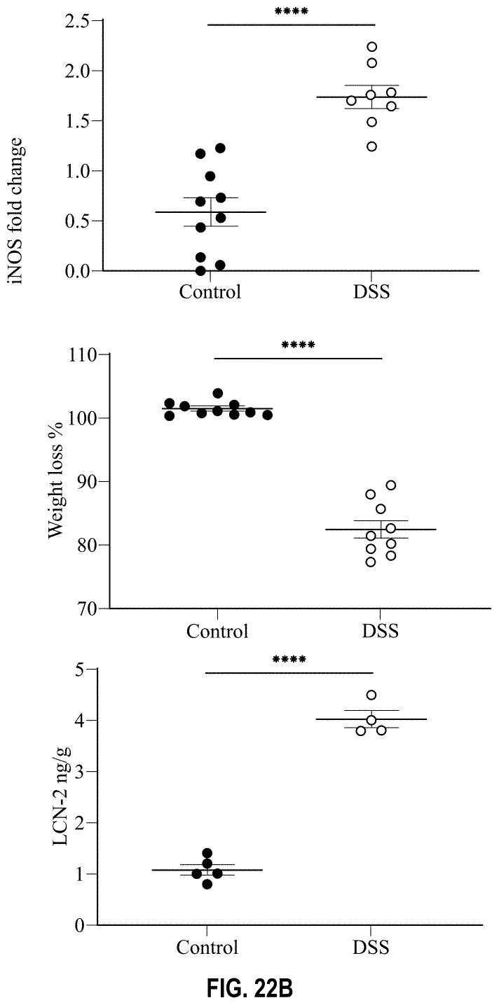

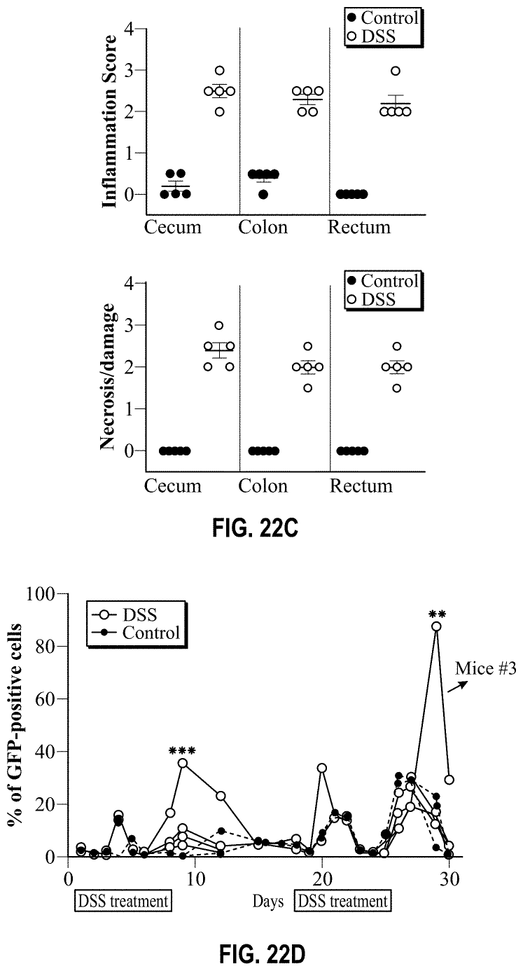

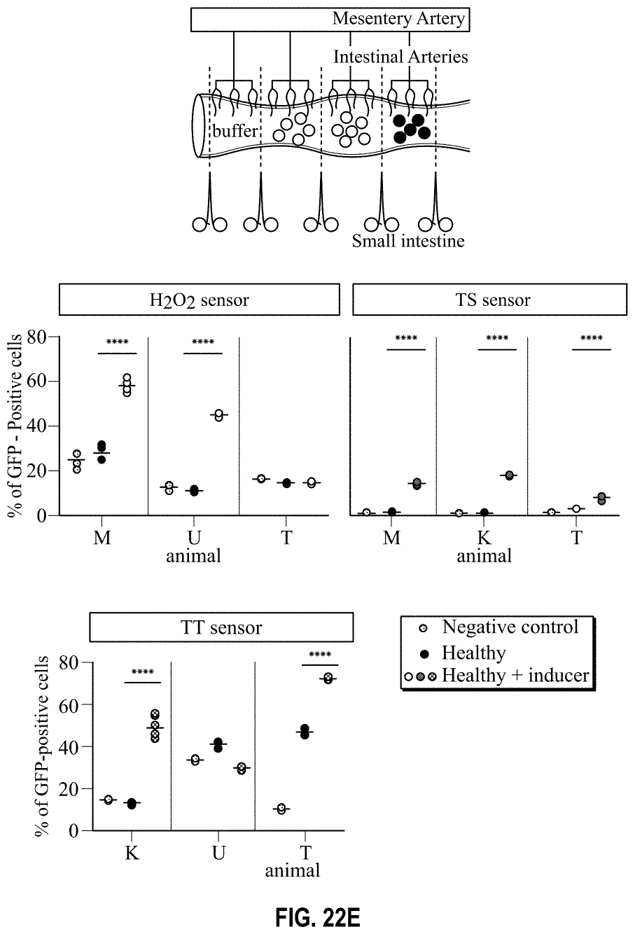

To examine functionality in vivo, the bacterial sensors were first evaluating if passing the bacterial sensors through the GI tract could detect chemically induced GI inflammation in a mouse model of colitis (FIG. 16C). After inflammation was induced with dextran sodium sulfate (DSS), control and treated mice were orally gavaged with the NO biosensors. After six hours, the percentage of GFP+ cells recovered from fecal samples was analyzed by flow cytometry. NO biosensors demonstrated significantly more GFP+ activation in fecal pellets from treated mice than in those from healthy controls (FIG. 16C). The novel biosensor design described in this example was thus able to detect the presence of NO as a marker of GI inflammation in vivo. Inflammation in the colitis model was measured over time (FIG. 22A) and independently validated (FIG. 22B-22C).

GFP activation increased significantly at day nine after the start of DSS treatment (FIG. 22A) correlating with the peak of nitric oxide synthase (iNOS) activation. Tracking NO with the DSD revealed an exacerbated inflammatory response following antibiotic treatment in a chronic inflammation model (FIG. 22D). The bacterial sensors were also tested for ROS (H₂O₂), TT and TS; activation was detected as inflammation progressed, with TS detection at day six after the start of DSS treatment and TT (TS product of oxidation) at day eight, which overlapped with ROS detection (FIG. 21A).

To validate the bacterial sensors in a disease model comparable in physiology and scale to human anatomy, the bacterial biosensors were also tested in pigs via an ischemia/reperfusion injury model of intestinal inflammation. The bacterial biosensors were injected directly into the intestinal lumen of a sedated animal, in either inflamed segments, or healthy segments with or without added biomarkers. The NO sensing bacteria could detect different concentrations of NO in the control-treated group (FIG. 16D) and lead to positive signal in the disease group (ischemia/repertusion, FIG. 16E); the ROS, TS, and TT sensors also detected significant quantities of analytes in control-treated pigs (FIG. 22E).

### Validation of an Integrated, Sub-1.4 mL, Ingestible Bacterial-Electronic Pill

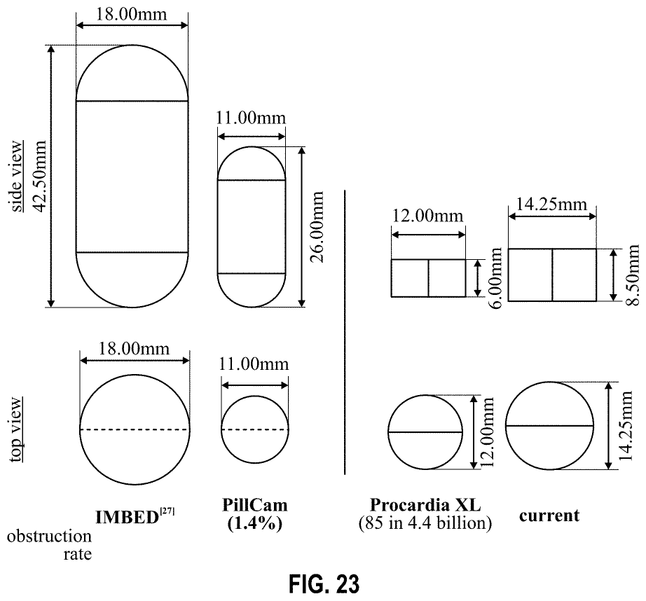

To advance the diagnostic potential of the bacterial sensors towards clinical application, the bacterial sensors were integrated into a bacterial-electronic pill. Specifically, a system was designed with a size and form factor conforming to a non-deformable dosage form (FIG. 23). To achieve this goal, an optimized luminescent readout, a custom microelectronic bioluminescence detector, and bacterial chambers built directly into the pill casing were developed.

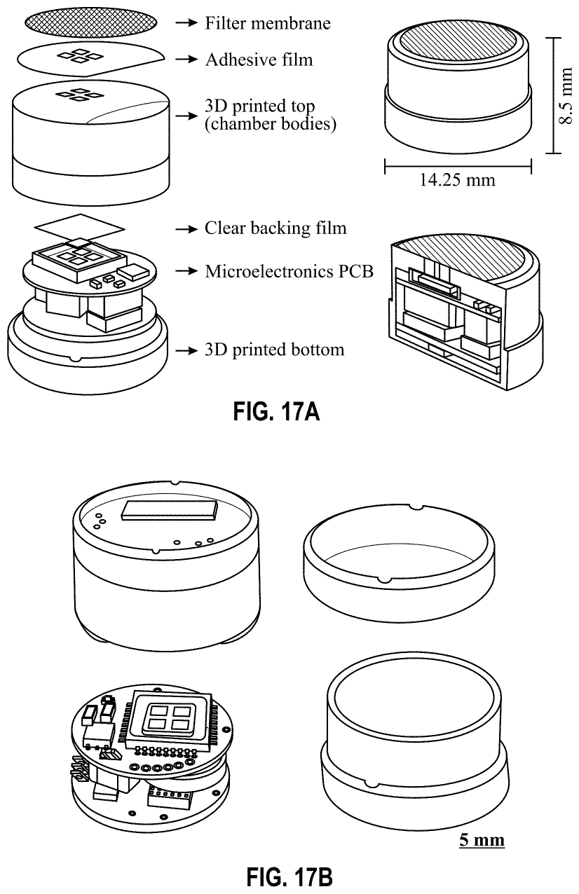

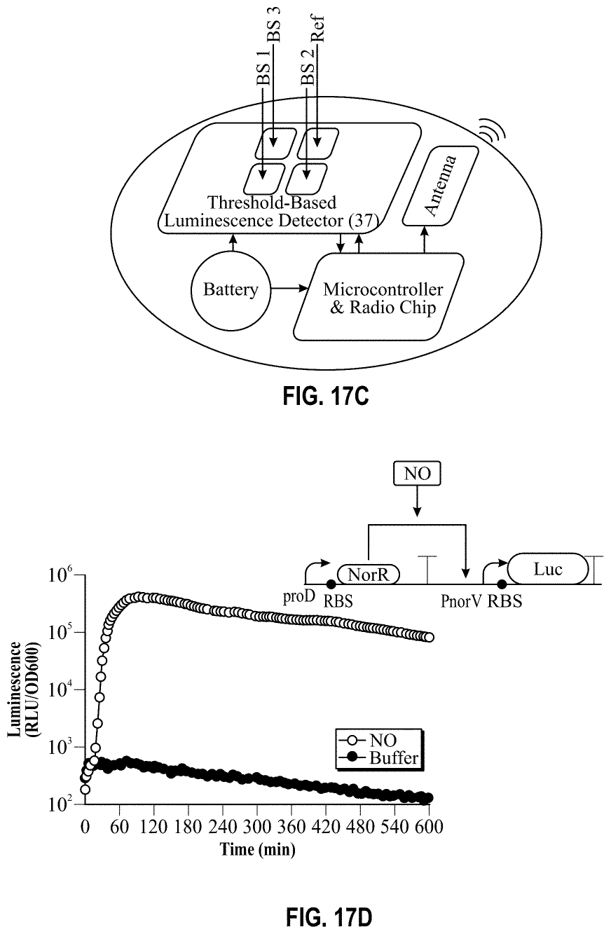

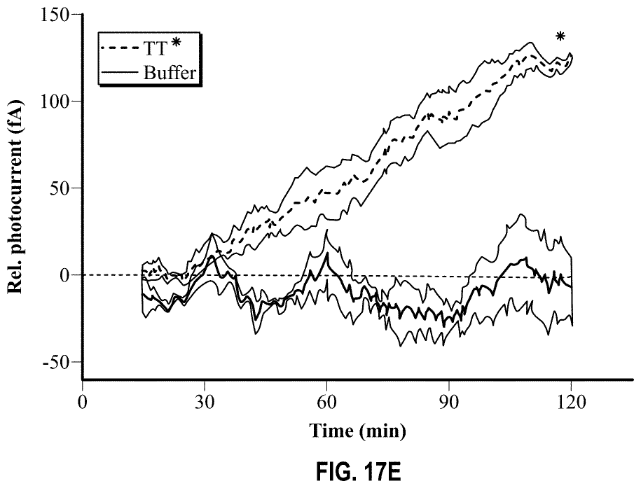

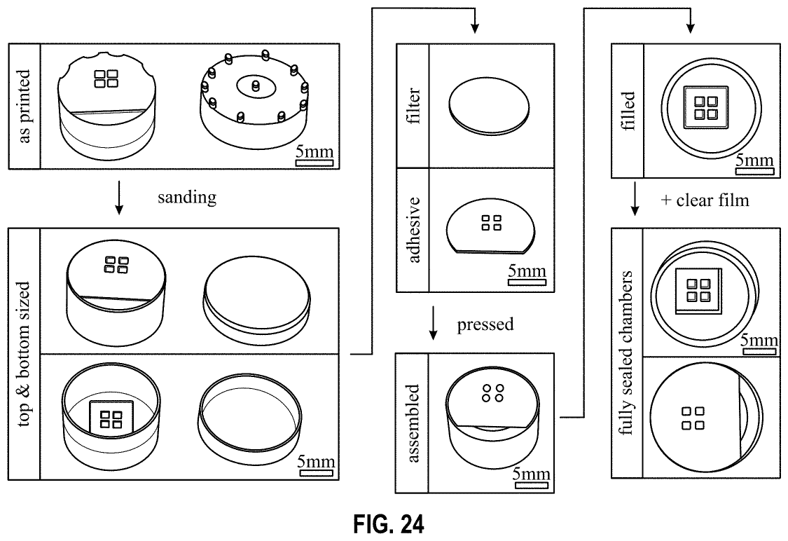

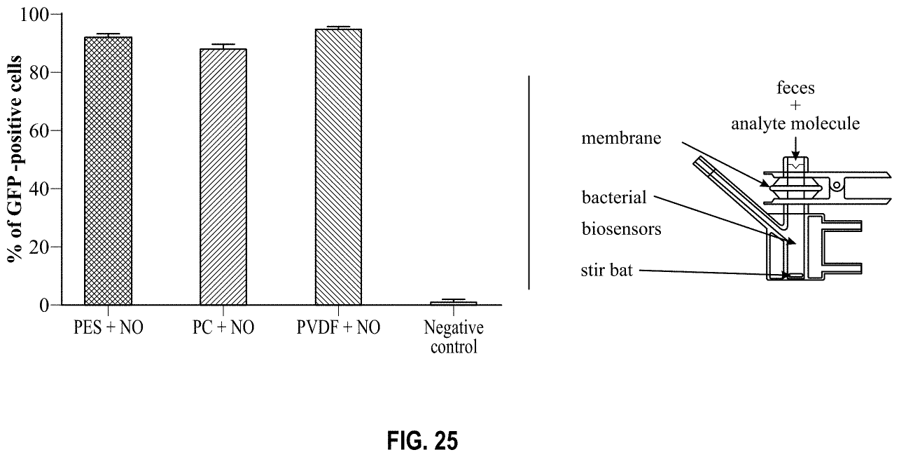

The multi-diagnostic device required nutrients and analytes to be exchanged efficiently while retaining the engineered microbes and simultaneously allowing the generated light signal to reach the electronic detectors. To meet this design requirement, a pill casing was developed that incorporates a bacterial-electronic chamber interface in a unibody design (FIG. 17A-17B). This small tablet-shaped housing precisely aligned the bacteria in a two-by-two array with the microelectronic photodiodes while maintaining a hermetic seal around the electronic components. This design enabled sensing from an array of engineered microbial strains designed to express luciferase in response to several biomarkers (e.g., NO, ROS, TT and negative reference(s)). The integrated chambers (FIG. 24) were sealed with porous membranes (e.g., a nominal pore size, 0.4 μm) to retain the bacterial cells. The porous membranes, when placed in feces, did not interfere with the detection of the target molecules (FIG. 25).

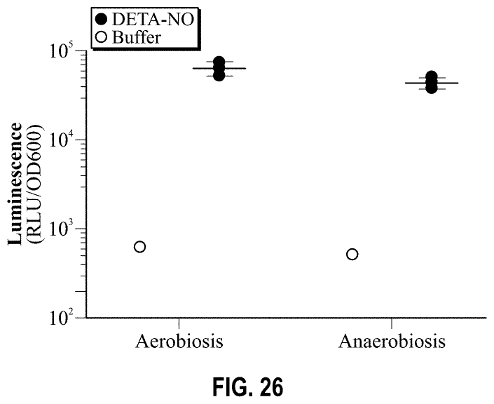

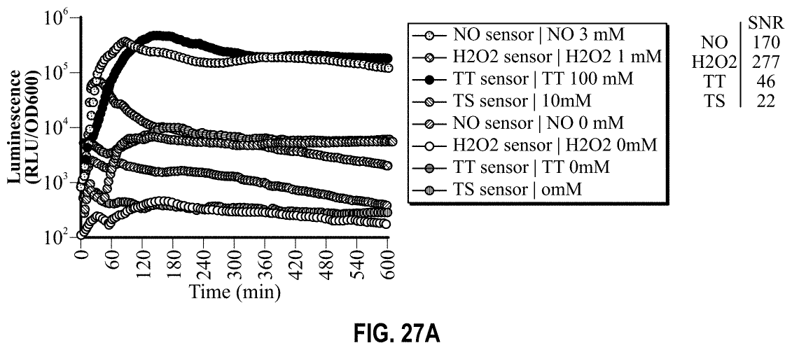

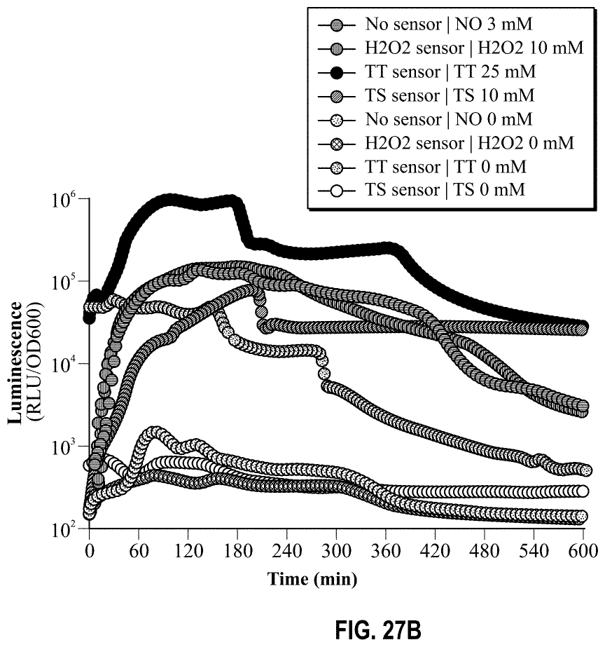

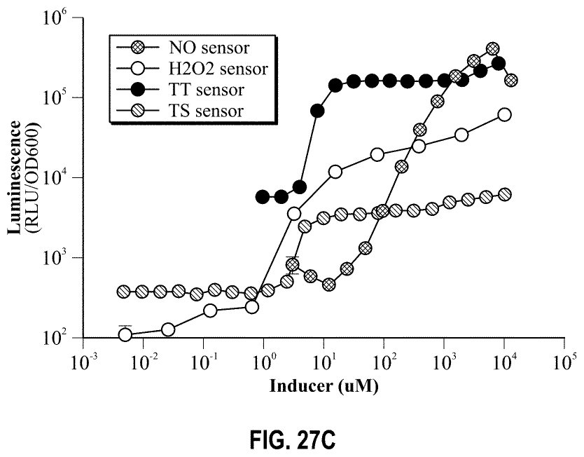

To generate bacterial sensors compatible with the photosensitive electronics, the GFP readout was replaced with a self-contained bioluminescence readout (e.g., the luxCDABE operon from Photorhabdus luminescens). This genetic circuit was constructed in E. coli Nissle 1917 and exposed the resultant strain to DETA/NO as a source of NO. The NO biosensing bacteria responded rapidly to DETA/NO exposure (tₘₐₓ=60 mins) with a high luminescence output and a SNR of 170 (FIG. 17C). Luminescence production was also induced by DETA/NO under anaerobic conditions (FIG. 26). The other inflammatory sensors were also built and characterized in vitro (FIG. 27A-27C), with TT sensor reaching the highest luminescence values in simulated intestinal fluid (FIG. 27B).

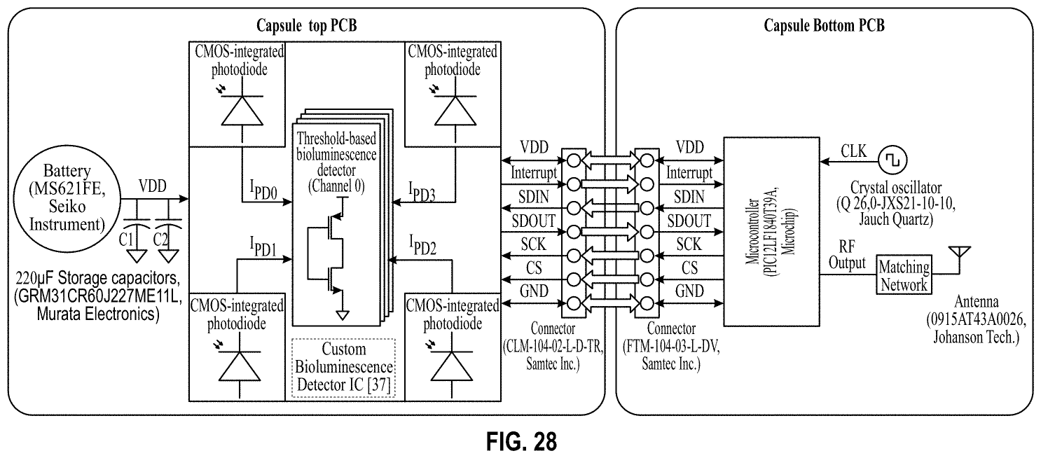

Lastly, a millimeter-scale capsule was designed on a printed circuit board (PCB) housing a complementary metal-oxide-semiconductor (CMOS) bioluminescence detector chip with an integrated photodiode array achieving high sensitivity (FIG. 17A). The custom chip integrated a threshold-based bioluminescence detector, time-to-digital converter, voltage references, voltage regulators, and four 1 mm by 1 mm CMOS-compatible photodiodes (FIG. 28). In addition to the custom chip, the ingestible capsule also included a commercial microcontroller and wireless transmitter. Bioluminescence from activated cells was detected by CMOS-integrated photodiodes located below each chamber. Custom-designed electronics processed the luminescence data by periodically sampling the photon-generated charge (e.g., with a programmable integration time of ˜26 s). The detected luminescence was converted to a digital code by the low-power luminescence readout chip and transmitted wirelessly for calibration, display, and recording.

When tested in vitro, using 1 μL of sensor bacteria culture per chamber, the integrated device successfully detected the presence of TT. A net 175 fA photocurrent was recorded, produced by the induced TI bacteria sensor, with a baseline of ˜0 fA from the uninduced control (FIG. 17D).

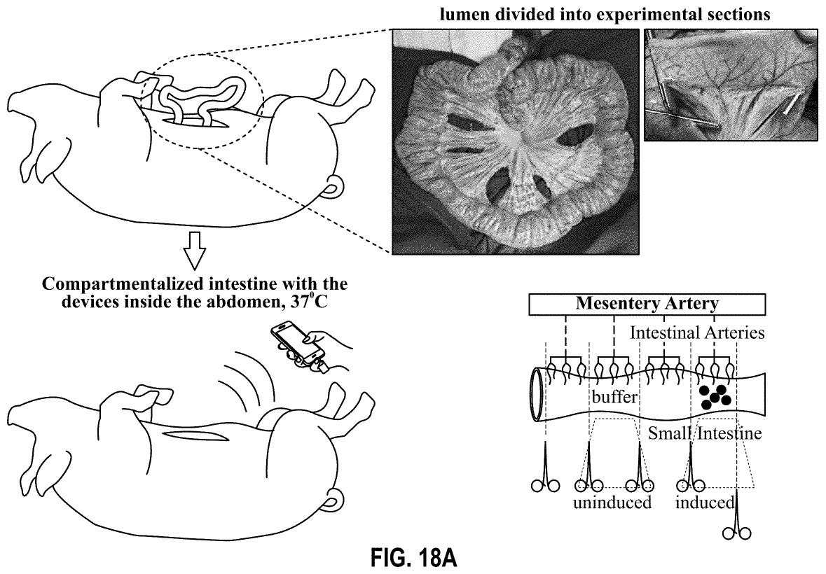

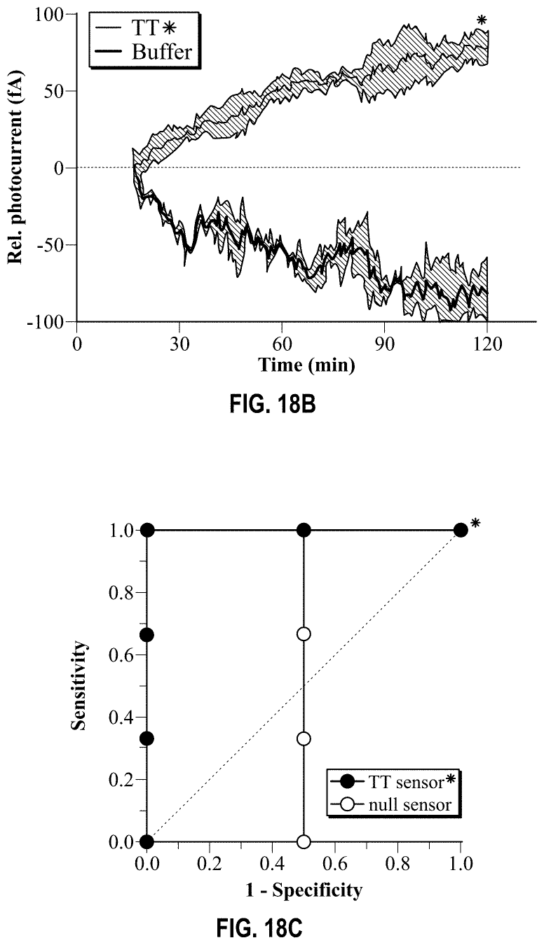

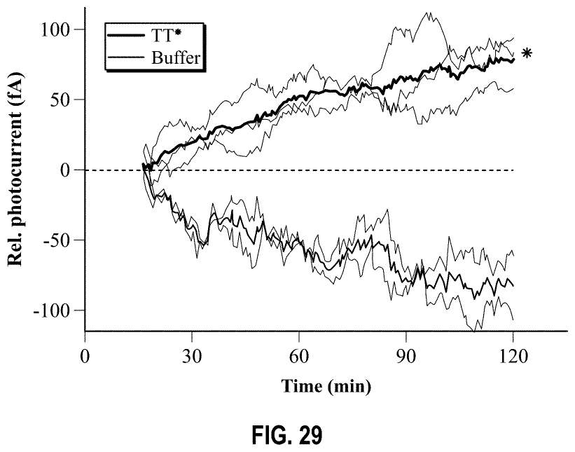

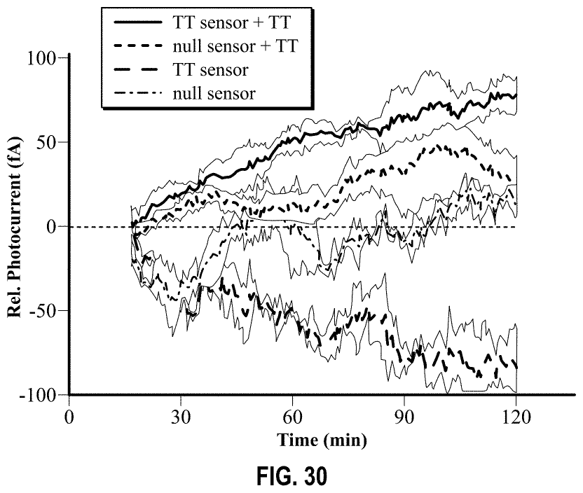

The integrated device was also tested in vivo in pig small intestines as a model for the complex milieu of the GI tract. Upon induction, luminescence was detected by the custom-designed electronic readout circuits in the capsule; the information was wirelessly transmitted in real time from inside the body of the pig to an external recording device (FIG. 18A). This design allowed remote monitoring of biomolecules in the gut for four hours, with 75 fA relative photocurrent detected in the induced compartment, and a baseline of −90 fA from the uninduced control (FIG. 18B and FIG. 29). The receiver operating characteristic of the TT sensing reached a sensitivity and specificity of 100% at 60 min. The miniaturized device could thus detect small amounts of analyte in the harsh intestinal environmental with relatively high specificity and sensitivity (FIG. 18C and FIG. 30).

### Discussion

Ingestible microbial biosensors were built that could sense an array of biomarkers in situ, as these biomarkers are being produced. This technology may be applied as a tool to support remote disease management by providing quantitative, real-time, and multiplexed information linking GI tract microbiome perturbations to disease. To validate the cell-based biosensors in preclinical disease models, engineered sensing bacteria were coupled with a recombinase-based memory system. The memory system recorded information as soon as metabolites are produced in the gut, activating switches within minutes of exposure. The recombinase-based switch discretizes the magnitude of a given biomarker; this quantitative response may align with disease stage and therefore indicate severity of inflammation. Although some of the parameters that can be measured with this device may have no absolute “healthy range,” measurements taken over time would reveal patterns predictive of acute disease episodes (flares); disease symptoms could then be anticipated. Similarly, the switch may act as a peak detector for sensing and recording maximum levels of intestinal biomarkers.

To create the miniaturized, low-power, ingestible electronic device, we integrated CMOS-compatible photodiodes with discrete-time signal processing circuits, which made it possible to miniaturize the whole capsule to a size below 1.4 cm³ and simultaneously detect multiple disease biomarkers on a stringent power budget. This integrated CMOS system combined with the unibody chamber pill casing design allowed for the detection of the bioluminescent signal from just 1 μL of bacterial culture in the milieu of the intestinal lumen. In addition, the coin-cell battery can power the ingestible capsule for a month, so this device could also potentially be used as an implant.

The diagnostic accuracy and specificity of the device was based on the simultaneous testing of an array of labile by-products of inflammation (e.g., NO and ROS), intestinal gases (e.g., H₂S measured as TS), and microbiome-derived biomolecules (e.g., TT). As biomarker levels may vary greatly among patients, a panel of biomarkers might be required to accurately diagnose IBD and other multi-faceted diseases.

The ingestible device offers a route for non-invasively evaluating changes in the intestinal biochemical milieu and overcomes the limitations of microbiome characterization by 16S rRNA or metagenomic sequencing, as well as current research applications of ingestible biosensors in animal models, which require the complex analysis of bacterial gene expression or RNA/DNA in stool. The biosensors described herein also have the potential to expand on the range of biomarkers being targeted by other ingestible electronic systems. The capsule may be designed to report its location while in transit and perform cell-based computation to further expand the multiplex capabilities of the underlying electronic system. For example, AND-gates may be incorporated to determine the co-localization of biomarkers for understanding metabolic pathways or biomarker discovery in animal models for many microbiome-linked diseases.

This device may be developed as a first-line at-home screen for non-invasive continuous monitoring of the chemical environment of the GI tract and customized for numerous GI disorders, thus offering a safe and inexpensive point-of-care alternative to, for example, imaging capsules for endoscopy. Tracking and quantitatively assessing multiple biomarkers may also provide a framework for patients to assess the effects of diet, lifestyle, and other interventions that require routine screening to improve health outcomes.

### Materials and Methods

### Bacterial Strains and Culture Conditions

Routine cloning and plasmid propagation were performed in E. coli E. cloni 10G (Lucigen). For in vitro and in vivo experiments, the probiotic strain E. coli Nissle 1917 was transformed with gene circuits built on plasmids. Cells were routinely cultured at 37° C. in Luria-Bertani (LB) media (Difco). Where appropriate, growth media was supplemented with antibiotics at the following concentrations: 50 μg/mL kanamycin, 100 μg/mL carbenicillin, 25 μg/mL chloramphenicol, and 100 μg/mL spectinomycin.

### Plasmid Construction and Circuit Characterization

Plasmids were constructed by combining PCR fragments generated by Phusion High-Fidelity DNA Polymerase (NEB) using Gibson Assembly, starting from DNA sources as referenced in Table 1-1 (further below) or from gBlocks manufactured by IDT. Some genetic parts and plasmids used in this example are listed in Table 1-1 and may be available from Addgene. Assembly products were introduced by transformation into chemically competent E. coli E. cloni 10G, and sequences were confirmed using Sanger sequencing.

To characterize the constructs built in conjunction with the memory system, appropriate antibiotics were added to Teknova Hi-Def Azure Media containing 0.2% glucose (w/v), and E. coli E. cloni 10G colonies were inoculated into this culture medium. Cultures were incubated aerobically with shaking for 16-18 hr at 37° C., then diluted 2,500× into fresh Hi-Def Azure Media (also containing appropriate antibiotics and 0.2% glucose (w/v)) and incubated aerobically with shaking for another 20 min at 37° C. Cultures (200 ml) were then transferred to a 96-well plate, and the respective inducers, H₂O₂ (hydrogen peroxide Sigma-Aldrich H1009-100ML), DETA/NO (diethylenetriamine/nitric oxide adduct DETA/NO (diethylenetriamine/nitric oxide adduct Sigma-Aldrich D185-50MG), TT (potassium tetrathionate Sigma-Aldrich P2926-100G), and TS (sodium thiosulfate Sigma-Aldrich 217263-250G), were added at appropriate concentrations via serial dilution. Plates were incubated either aerobically with shaking or anaerobically for 20 h at 37° C. for the experiments. For experiments performed in anaerobic conditions, cultures were grown and manipulated in a Coy anaerobic chamber with an atmosphere of 85% N₂, 5% H₂ and 10% CO₂ at 37° C. Media was pre-reduced overnight in anaerobic atmosphere before inoculation of cultures. After incubation, the optical densities of cultures were measured at 600 nm in a plate reader.

For flow cytometry, cells were diluted in cold 1×PBS and 2% sucrose to reach an optical density of 0.02 (at 600 nm), then assayed on a BD LSRFortessa. The “FITC” channel was used to measure GFP expression. A minimum of 10,000 gated events was recorded. FlowJo software was used to export and process FCS files. Forward scatter and side scatter were used to gate for live cells as described previously. On the resulting flow cytometry histograms, the y-axis is normalized to the mode for each sample. Each experiment was performed for at least three biological replicates.

### Growth and Induction

For genetic circuit characterization, overnight cultures were diluted 1:100 in fresh LB and incubated with shaking at 37° C. for 2 hr. Cultures were removed from the incubator and 200 μL of culture was transferred to a 96-well plate containing various concentrations of inducer. The plate was returned to a shaking incubator at 37° C. Following 2 hr of incubation, luminescence was read using a BioTek Synergy H1 Hybrid Reader with a is integration time and a sensitivity of 150. Luminescence values, measured in relative luminescence units (RLUs), were normalized by the optical density of the culture measured at 600 nm.

For in vitro kinetic studies, subcultured cells were mixed with an inducer in a 96-well plate and immediately placed in the plate reader set at 37° C. without shaking. Luminescence and absorbance were read at 3-minute intervals.

### Mouse Experiments

Approval for mouse experiments was obtained from the Committee on Animal Care at the Massachusetts Institute of Technology. Male C57BL/6J mice (8-10 weeks of age) were obtained from Jackson Labs (Stock No: 000664). Conventional conditions were used to house and handle the mice. One week before the experiments began, the mice were acclimated to the animal facility.

Animals were randomly allocated to experimental groups. Researchers were not blinded to group assignments. Overnight cultures of E. coli Nissle grown in Teknova Hi-Def Azure Media with appropriate antibiotics and 0.2% glucose (w/v) were centrifuged at 5000 g for 5 minutes and resuspended in an equal volume of 20% sucrose. Animals were inoculated with 200 μL of bacteria culture (approximately 10⁸ CFU) by oral gavage. Fecal pellets were collected 6 hr post-gavage and homogenized in 1 mL of PBS with a 5 mm stainless steel bead using a TissueLyser II (Qiagen) at 25 Hz for 2 minutes. Samples were centrifuged at 500 g for 30 seconds to pellet large fecal debris. Supernatant was cultured in Teknova Hi-Def Azure Media with appropriate antibiotics and 0.2% glucose (w/v) and incubated aerobically with shaking for 16-18 hr at 37° C. Cells were then assayed on the flow cytometer.

### Pig Experiments

Approval for pig experiments was obtained from the Committee on Animal Care at the Massachusetts Institute of Technology. Female Yorkshire pigs (50-95 kg), received from Cummings Veterinary School at Tufts University in Grafton, Mass., were randomly selected for the experiments and housed under conventional conditions. Prior to the experiment, animals were given a clear liquid diet for 24 hours. The day of the experiment, the morning feed was withheld. Pigs were sedated with Telazol® (tiletamine/zolazepam 5 mg/kg), xylazine (2 mg/kg), and atropine (0.04 mg/kg) at the start of the experiment. The jejunum was accessed via a midline laparotomy and the lumen sectioned into several test compartments using Mayo-Robson intestinal clamps. Ischemia-reperfusion injury was used as a model of intestinal inflammation and was caused by clamping the mesentery of the target intestinal segment with hemostatic clamps for 2 hours and then releasing the clamps to allow reperfusion for at least 1 hour. At the end of the procedure pigs were euthanized with Fatal Plus (sodium pentobarbital): 1 ml/10 lbs or approximately 115-120 mg/kg and heart rate assessed to ensure the pig was euthanized. For bacteria-only experiments, overnight bacterial cultures were diluted 1:10 in LB, cultured for 20 mins, resuspended in 1 mL PBS after centrifugation and was injected into the target intestinal section via a syringe. Healthy intestinal sections were used as is or injected with 200 μL of the target analyte (DETA/NO, H₂O₂, TT, TS) as described elsewhere herein. After the experiment, cells were retrieved by flushing the intestinal section with 10 mL of PBS injected and retrieved via a syringe. For device-bacteria experiments, overnight bacterial cultures were diluted 1:10 in LB, cultured for 20 mins and 1 μL of fresh culture (concentrated 100× by centrifugation) was used to fill the pill casing chambers and sealed as described below (Pill casing manufacture).

Devices were inserted into the intestinal lumen through a small incision, manually passed into the target intestinal section and isolated from the incision site by luminal clamping as described above. TT (100 mM) was injected via a syringe into the clamped intestinal compartment and data from the capsules was wirelessly acquired via a 915 MHz radio attached to a laptop. Devices were manually retrieved from the jejunum. A total of 3 animals were included in the experiments; 3 animals on different days, 2 intestinal sections per animal, one for administering the inducer molecule and the other compartment as negative control.

### Preparation of Electronic Components

The electronics in the capsules consisted of four photodiodes (Integrated CMOS P+/NWELL/PSUB photodiodes), a custom bioluminescence detector chip fabricated in a CMOS 65 nm process, a microcontroller (PIC12LF1840T39A, Microchip) and radio chip (PIC12LF1840T39A, Microchip Technology Inc.), and a 915 MHz chip antenna (0915AT43A0026, Johanson Technology). A commercial receiver (CC1200, Texas Instruments) was also used. The upper side of the top printed circuit boards (PCB) held the fully quartz lid-packaged CMOS chip together with an additional on-board LDO (ADP-166, Analog Devices). The assembly was coated with 1 μm of Parylene C to act as a moisture barrier for the electronic components. Parylene C coating was performed using Specialty Coating System Labcoter 2 (PDS 2010) with 1 gram of dichlorodi-p-xylene to reach a target layer thickness of 1 μm using a known protocol.

### Pill Casing Manufacture

Pill casing top and bottom blanks were printed vat photopolymerization (e.g., laser printing) and then flat outer faces were sanded to size. In the final design, an Isopore membranes (0.4 μm pore size, Millipore-Sigma) were cut to size with a punch and then attached to the casing body via a thin, lasercut double-sided adhesive layer (3M VHB 5906). Unfilled pill casing tops were conditioned for 24 hours in LB broth, and on the day of the experiment, the chambers were filled from the inside face with bacterial suspensions and subsequently sealed with a thin, lasercut, optically clear adhesive backing film (GeneMate Polyolefin Films with Silicone Adhesive). Bacteria-filled casing tops and empty bottoms were then press fit around the electronic system and the outer seam waterproofed with additional silicone (Elite Double 32, Zhermack). Porous membrane types were initially screened as described in FIG. 24 using a standard two-chamber Franz cell.

### In Vitro Device Measurements

LB culture media was pre-warmed for at least 2 hr prior to the start of experiments. For device-bacterial experiments, overnight cultures were diluted 1:10 in LB, subcultured for 20 min, and concentrated 100× by centrifugation. 1 μL of the concentrated culture was then added to pill casing chambers. Wild-type E. coli Nissle 1917 was added to the reference channel for the experiments. Once the four channels were loaded, the cell carrier was fastened to the capsule and fully submerged in pre-warmed media. LB culture media supplemented with inducer (100 mM TT, 20 mM NO, 1 mM H₂O₂ or 100 mM TS). Cultures were wrapped several times in thick black fabric to block external light and placed in an incubator at 37° C., and data was collected wirelessly for 2 hr. At the end of the experiment, devices were dissembled, and cell carriers were discarded. Capsules were sterilized with 70% ethanol and thoroughly washed with distilled water. Capsules were left to air-dry and re-used for future experiments.

### Calibration Procedure for Converting Detector Counts to Estimated Photocurrent

One-time optical calibration was performed to obtain the custom integrated circuit (IC) performance and calibration parameters. During the optical calibration, the wireless capsule holding the custom luminescence detector IC, a standalone photodiode IC, and a green LED (λ=520 nm) were placed inside a metal box covered by a blackout cloth to prevent ambient light from the environment. The capsule and the standalone photodiode IC were placed 1.5 cm adjacent to each other on one side of the metal box, while the LED was placed 30 cm opposite to both Ics on the other side of the metal box. Five different voltage levels were applied (0 V, 2.1 V, 2.14 V, 2.165 V, 2.185 V) across the LED to obtain different optical power levels. The custom luminescence detector IC wirelessly transmitted sensor readout Nᵢ for each sensing channel i at a given optical power. The standalone photodiode IC was exposed to this optical power simultaneously, and the IC reported a photocurrent level I_{PD} through a sub-femtoamp SourceMeter (K6430, Keithley Instruments). The resolution of a sensing channel i is defined as:

$$Resolution = \frac{Δ {I}_{PD}}{Δ {N}_{i}}$$

where ΔI_{PD} is the difference between photocurrent values reported from the standalone photodiode IC for two LED bias voltages; and ΔNᵢ is the difference between luminescence detector IC output counts for the same LED bias voltages. The resolution for a single channel was calculated using a linear regression model to fit the luminescence IC output counts and photocurrents from standalone photodiode IC over five LED bias voltages. The resolution of the luminescence detector chip is 5.8-6.5 fA/count. The minimum detectable signal at one LED bias voltage was calculated using the resolution of each sensing channel times 1σ standard deviation of the IC output at this optical power. The worst-case minimum detectable signal is 71 fA. The estimated photocurrents for in-vitro and in-vivo measurements were calculated by taking the product of resolutions from one-time optical calibration and measured IC output counts in each measurement. We used a moving average filter of 25 samples (˜15 min moving average).

### Data Analysis, Statistics and Computational Methods

Data were analyzed using GraphPad Prism version 9.1.2 (Graph Software, San Diego, Calif., USA, www.graphpad.com). Sequences were analyzed using SnapGene version 5.1.2 (www.snapgene.com). As noted, error bars represent the standard error of the mean (SEM) of at least three independent experiments carried out on different days. Significant differences between groups were determined using an unpaired, two-tailed Student's t-test assuming unequal variance and for curves over time, two-way ANOVA for multiple comparisons. Fold change or signal-to-noise ratio was determined by dividing the normalized luminescence values (RLU/OD600) of samples treated with the maximal inducer concentration with uninduced samples. The receiver operating characteristic was calculated at based on three independent experiments.

The table provides particular DNA sequence listings for certain parts.

TABLE 1-1
Genetic Parts

| Part | | SEQ |
|---|---|---|
| Name | DNA sequence | ID NO: |
| oxySp | TTCATTATCCATCCTCCATCGCCACGATAGTTCATGGCGAT | 1 |
| | AGGTAGAATAGCAATGAACGATTATCCCTATCAAGCATTC | |
| | TGACTGAGCATTGCTCACA | |
| oxyR | ATGAATATTCGTGATCTTGAGTACCTGGTGGCATTGGCTG | 2 |
| | AACACCGCCATTTTCGGCGTGCGGCAGATTCCTGCCACGT | |
| | TAGCCAGCCGACGCTTAGCGGGCAAATTCGTAAGCTGGAA | |
| | GATGAGCTGGGCGTGATGTTGCTGGAGCGGACCAGCCGTA | |
| | AAGTGTTGTTCACCCAGGCGGGAATGCTGCTGGTGGATCA | |
| | GGCGCGTACCGTGCTGCGTGAGGTGAAAGTCCTTAAAGAG | |
| | ATGGCAAGCCAGCAGGGCGAGACGATGTCCGGACCGCTG | |
| | CACATTGGTTTGATTCCCACAGTTGGACCGTACCTGCTACC | |
| | GCATATTATCCCTATGCTGCACCAGACCTTTCCAAAGCTGG | |
| | AAATGTATCTGCATGAAGCACAGACCCACCAGTTACTGGC | |
| | GCAACTGGACAGCGGCAAACTCGATTGCGTGATCCTCGCG | |
| | CTGGTGAAAGAGAGCGAAGCATTCATTGAAGTGCCGTTGT | |
| | TTGATGAGCCAATGTTGCTGGCTATCTATGAAGATCACCC | |
| | GTGGGCGAACCGCGAATGCGTACCGATGGCCGATCTGGCA | |
| | GGGGAAAAACTGCTGATGCTGGAAGATGGTCACTGTTTGC | |
| | GCGATCAGGCAATGGGTTTCTGTTTTGAAGCCGGGGCGGA | |
| | TGAAGATACACACTTCCGCGCGACCAGCCTGGAAACTCTG | |
| | CGCAACATGGTGGCGGCAGGTAGCGGGATCACTTTACTGC | |
| | CAGCGCTGGCTGTGCCGCCGGAGCGCAAACGCGATGGGGT | |
| | TGTTTATCTGCCGTGCATTAAGCCGGAACCACGCCGCACT | |
| | ATTGGCCTGGTTTATCGTCCTGGCTCACCGCTGCGCAGCCG | |
| | CTATGAGCAGCTGGCAGAGGCCATCCGCGCAAGAATGGAT | |
| | GGCCATTTCGATAAAGTTTTAAAACAGGCGGTTTAA | |
| gfp | ATGAGTAAAGGAGAAGAACTTTTCACTGGAGTTGTCCCAA | 3 |
| | TTCTTGTTGAATTAGATGGTGATGTTAATGGGCACAAATTT | |
| | TCTGTCAGTGGAGAGGGTGAAGGTGATGCAACATACGGA | |
| | AAACTTACCCTTAAATTTATTTGCACTACTGGAAAACTACC | |
| | TGTTCCATGGCCAACACTTGTCACTACTTTCGGTTATGGTG | |
| | TTCAATGCTTTGCGAGATACCCAGATCATATGAAACAGCA | |
| | TGACTTTTTCAAGAGTGCCATGCCCGAAGGTTATGTACAG | |
| | GAAAGAACTATATTTTTCAAAGATGACGGGAACTACAAGA | |
| | CACGTGCTGAAGTCAAGTTTGAAGGTGATACCCTTGTTAA | |
| | TAGAATCGAGTTAAAAGGTATTGATTTTAAAGAAGATGGA | |
| | AACATTCTTGGACACAAATTGGAATACAACTATAACTCAC | |
| | ACAATGTATACATCATGGCAGACAAACAAAAGAATGGAA | |
| | TCAAAGTTAACTTCAAAATTAGACACAACATTGAAGATGG | |
| | AAGCGTTCAACTAGCAGACCATTATCAACAAAATACTCCA | |
| | ATTGGCGATGGCCCTGTCCTTTTACCAGACAACCATTACCT | |
| | GTCCACACAATCTGCCCTTTCGAAAGATCCCAACGAAAAG | |
| | AGAGACCACATGGTCCTTCTTGAGTTTGTAACAGCTGCTG | |
| | GGATTACACATGGCATGGATGATCTCTACAAATAA | |
| Bxb1 | ATGAGAGCCCTGGTAGTCATCCGCCTGTCCCGCGTCACCG | 4 |
| | ATGCTACGACTTCACCGGAGCGTCAGCTGGAGTCTTGCCA | |
| | GCAGCTCTGCGCCCAGCGCGGCTGGGACGTCGTCGGGGTA | |
| | GCGGAGGATCTGGACGTCTCCGGGGCGGTCGATCCGTTCG | |
| | ACCGGAAGCGCAGACCGAACCTGGCCCGGTGGCTAGCGTT | |
| | CGAGGAGCAACCGTTCGACGTGATCGTGGCGTACCGGGTA | |
| | GACCGGTTGACCCGATCGATCCGGCATCTGCAGCAGCTGG | |
| | TCCACTGGGCCGAGGACCACAAGAAGCTGGTCGTCTCCGC | |
| | GACCGAAGCGCACTTCGATACGACGACGCCGTTTGCGGCG | |
| | GTCGTCATCGCGCTTATGGGAACGGTGGCGCAGATGGAAT | |
| | TAGAAGCGATCAAAGAGCGGAACCGTTCGGCTGCGCATTT | |
| | CAATATCCGCGCCGGGAAATACCGAGGATCCCTGCCGCCG | |
| | TGGGGATACCTGCCTACGCGCGTGGACGGGGAGTGGCGGC | |
| | TGGTGCCGGACCCTGTGCAGCGAGAGCGCATCCTCGAGGT | |
| | GTATCACCGCGTCGTCGACAACCACGAGCCGCTGCACCTG | |
| | GTGGCCCACGACCTGAACCGGCGTGGTGTCCTGTCGCCGA | |
| | AGGACTACTTCGCGCAGCTGCAAGGCCGCGAGCCGCAGG | |
| | GCCGGGAGTGGTCGGCTACCGCGCTGAAGCGATCGATGAT | |
| | CTCCGAGGCGATGCTCGGGTACGCGACTCTGAACGGTAAG | |
| | ACCGTCCGAGACGACGACGGAGCCCCGCTGGTGCGGGCTG | |
| | AGCCGATCCTGACCCGTGAGCAGCTGGAGGCGCTGCGCGC | |
| | CGAGCTCGTGAAGACCTCCCGGGCGAAGCCCGCGGTGTCT | |
| | ACCCCGTCGCTGCTGCTGCGGGTGTTGTTCTGTGCGGTGTG | |
| | CGGGGAGCCCGCGTACAAGTTCGCCGGGGGAGGACGTAA | |
| | GCACCCGCGCTACCGCTGCCGCTCGATGGGGTTCCCGAAG | |
| | CACTGCGGGAACGGCACGGTGGCGATGGCCGAGTGGGAC | |
| | GCGTTCTGCGAGGAGCAGGTGCTGGATCTGCTCGGGGACG | |
| | CGGAGCGTCTGGAGAAAGTCTGGGTAGCCGGCTCGGACTC | |
| | CGCGGTCGAACTCGCGGAGGTGAACGCGGAGCTGGTGGA | |
| | CCTGACGTCGCTGATCGGCTCCCCGGCCTACCGGGCCGGC | |
| | TCTCCGCAGCGAGAAGCACTGGATGCCCGTATTGCGGCGC | |
| | TGGCCGCGCGGCAAGAGGAGCTGGAGGGTCTAGAGGCTC | |
| | GCCCGTCTGGCTGGGAGTGGCGCGAGACCGGGCAGCGGTT | |
| | CGGGGACTGGTGGCGGGAGCAGGACACCGCGGCAAAGAA | |
| | CACCTGGCTTCGGTCGATGAACGTTCGGCTGACGTTCGAC | |
| | GTCCGCGGCGGGCTGACTCGCACGATCGACTTCGGGGATC | |
| | TGCAGGAGTACGAGCAGCATCTCAGGCTCGGCAGCGTGGT | |
| | CGAACGGCTACACACCGGGATGTCGAGGCCTGCAGCAAA | |
| | CGACGAAAACTACGCTGCAGCAGTTTAG | |
| ProD | | |
| J23104 | TTGACAGCTAGCTCAGTCCTAGGTATTGTGCTAGCCTAGTA | 5 |
| | TCGATCTCCATAACTATCCTATAGATC | |
| J23105 | TTTACGGCTAGCTCAGTCCTAGGTACTATGCTAGCAGAAA | 6 |
| | TATAAAGAACGATCTATTTATCCGCGTAC | |
| ThsS | ATGTCCCGCCTGCTGCTGTGTATCTGTGTTCTGCTGTTCTCT | 7 |
| | TCTGTGGCGTGGTCTAAACCGCAGCAGTTTTATGTGGGCG | |
| | TACTGGCTAACTGGGGTCATCAGCAAGCCGTTGAACGTTG | |
| | GACCCCGATGATGGAGTATCTGAACGAACATGTGCCGGAC | |
| | GCGGAATTTCACGTCTACCCGGGCAACTTCAAAGCACTGA | |
| | ACCTGGCAATGGAACTGGGCCAGATTCAGTTCATTATCAC | |
| | TAACCCGGGCCAATATCTGTACCTGAGCAATCAGTACCCG | |
| | CTGTCTTGGCTGGCGACCATGCGTTCTAAGCGTCACGATG | |
| | GTACCACTTCTGCGATCGGTTCCGCCATTATTGTCCGCGCG | |
| | GACAGCGACTACCGCACCCTGTACGACCTGAAAGGTAAAG | |
| | TGGTGGCTGCGTCCGACCCGCATGCTCTGGGTGGCTACCA | |
| | AGCGACCGTCGGTCTGATGCATTCCCTGGGCATGGATCCG | |
| | GACACCTTCTTCGGTGAAACCAAGTTTCTGGGCTTTCCACT | |
| | GGATCCGCTGCTGTACCAAGTTCGTGATGGCAACGTTGAC | |
| | GCGGCCATTACCCCACTGTGCACTCTGGAGGACATGGTTG | |
| | CACGCGGCGTACTGAAATCTTCCGATTTTCGTGTGCTGAAC | |
| | CCTAGCCGCCCGGATGGTGTAGAATGCCAGTGCTCTACCA | |
| | CCCTGTACCCGAACTGGTCTTTCGCTGCGACTGAGTCTGTA | |
| | TCCACCGAACTGTCTAAAGAAATCACGCAGGCACTGCTGG | |
| | AACTGCCATCCGACAGCCCGGCAGCTATCAAAGCGCAACT | |
| | GACCGGCTGGACCAGCCCGATCTCCCAACTGGCGGTAATC | |
| | AAACTGTTCAAAGAGCTGCACGTAAAAACCCCGGACTCTA | |
| | GCCGTTGGGAAGCCGTTAAGAAGTGGCTGGAAGAAAACC | |
| | GTCACTGGGGTATCCTGTCTGTTCTGGTGTTCATCATTGCA | |
| | ACGCTGTATCACCTGTGGATTGAATACCGCTTCCACCAAA | |
| | AAAGCTCTTCTCTGATCGAATCTGAACGTCAGCTGAAACA | |
| | GCAAGCTGTTGCCCTGGAACGTCTGCAATCTGCTAGCATC | |
| | GTTGGTGAAATTGGTGCGGGTCTGGCCCACGAGATTAATC | |
| | AGCCGATCGCTGCAATTACCTCTTATTCTGAAGGTGGCATC | |
| | ATGCGCCTGCAAGGTAAAGAACAGGCGGATACGGATAGC | |
| | TGCATCGAACTGCTGGAAAAAATCCACAAACAGAGCACTC | |
| | GCGCAGGCGAAGTGGTGCACCGCATCCGTGGTCTGCTGAA | |
| | ACGTCGTGAAGCGGTGATGGTAGATGTTAACATCCTGACC | |
| | CTGGTGGAAGAATCCATCAGCCTGCTGCGTCTGGAGCTGG | |
| | CACGTCGCGAAATCCAGATCAACACTCAGATCAAAGGTGA | |
| | ACCGTTCTTCATTACTGCCGACCGCGTTGGCCTGCTGCAAG | |
| | TTCTGATTAACCTGATCAAAAACTCCCTGGACGCGATCGC | |
| | TGAATCTGATAATGCCCGTTCTGGTAAAATCAACATCGAA | |
| | CTGGACTTTAAAGAGTACCAGGTAAACGTCTCCATCATCG | |
| | ATAACGGTCCGGGCCTGGCGATGGATTCTGACACTCTGAT | |
| | GGCTACGTTTTACACTACCAAAATGGATGGCCTGGGCCTG | |
| | GGTCTGGCAATCTGCCGCGAAGTTATCAGCAACCACGACG | |
| | GCCACTTCCTGCTGTCCAACCGTGACGACGGCGTTCTGGG | |
| | CTGTGTGGCAACCCTGAATCTGAAAAAACGCGGTTCTGAA | |
| | GTGCCGATCGAAGTCTAA | |
| ThsR | ATGCAGCAGCAAATCAACGGCCCGGTCTACCTGGTGGATG | 8 |
| | ATGATGAAGCCATTATCGACTCCATCGATTTTTTGATGGAG | |
| | GGCTACGGTTACAAACTGAACTCGTTTAACTGCGGCGATC | |
| | GCTTTTTGGCAGAAGTCGATCTCACCCAGGCAGGATGTGT | |
| | AATTCTGGATGCGCGTATGCCAGGCTTAACTGGTCCTCAG | |
| | GTGCAACAGCTGCTGAGCGACGCGAAAAGCCCGCTTGCGG | |
| | TCATCTTCCTGACCGGCCATGGCGATGTTCCGATGGCGGTT | |
| | GATGCGTTCAAAAATGGCGCGTTCGATTTCTTTCAAAAAC | |
| | CTGTGCCGGGTAGCTTGCTCAGTCAGTCAATTGCCAAAGG | |
| | CTTGACTTATTCAATCGATCAACATCTGAAACGTACTAACC | |
| | AAGCGTTAATCGACACGCTCTCGGAACGCGAAGCTCAAAT | |
| | TTTTCAACTGGTGATTGCAGGCAACACCAACAAACAGATG | |
| | GCTAACGAGCTTTGCGTGGCTATTCGTACCATTGAGGTTCA | |
| | CCGTAGCAAACTGATGACCAAACTGGGTGTTAACAACCTG | |
| | GCTGAACTGGTTAAACTGGCGCCGCTGCTGGCACATAAAT | |
| | CCGAATAA | |
| PphsA | TTCAAGCATTATTATGCTGTTTTTTGAAGTGAATGTGCGGC | 9 |
| | CATCTAGCCGCACATTTTGCATCTAAAACATGCAGTCATC | |
| | AGCAAAATAATAAACTTTTCCCCAATATGTGGTTTACCAC | |
| | AATTTACAGGAATTCACTCCTGTGGTGGTGCAAATTTGAA | |
| | CTGTGAATTGCTTCACAAACGCCGCTATCGCAATGTCAGT | |
| | ATGTGGTTTACCACAATATCTAATATCACTCTGCTCAATAA | |
| | CAATGATGAAAACCTTAGGAAGAAGTTAATTGTGTTAAAC | |
| | AGTTAACTAGGGGCTTTATCTAACGCTCTCCTAAGGACAA | |
| | CTGTCATTGGGAGATTTAAC | |
| LuxC | ATGACTAAAAAAATTTCATTCATTATTAACGGCCAGGTTG | 10 |
| DABE | AAATCTTTCCCGAAAGTGATGATTTAGTGCAATCCATTAAT | |
| | TTTGGTGATAATAGTGTTTACCTGCCAATATTGAATGACTC | |
| | TCATGTAAAAAACATTATTGATTGTAATGGAAATAACGAA | |
| | TTACGGTTGCATAACATTGTCAATTTTCTCTATACGGTAGG | |
| | GCAAAGATGGAAAAATGAAGAATACTCAAGACGCAGGAC | |
| | ATACATTCGTGACTTAAAAAAATATATGGGATATTCAGAA | |
| | GAAATGGCTAAGCTAGAGGCCAATTGGATATCTATGATTT | |
| | TATGTTCTAAAGGCGGCCTTTATGATGTTGTAGAAAATGA | |
| | ACTTGGTTCTCGCCATATCATGGATGAATGGCTACCTCAG | |
| | GATGAAAGTTATGTTCGGGCTTTTCCGAAAGGTAAATCTG | |
| | TACATCTGTTGGCAGGTAATGTTCCATTATCTGGGATCATG | |
| | TCTATATTACGCGCAATTTTAACTAAGAATCAGTGTATTAT | |
| | AAAAACATCGTCAACCGATCCTTTTACCGCTAATGCATTA | |
| | GCGTTAAGTTTTATTGATGTAGACCCTAATCATCCGATAAC | |
| | GCGCTCTTTATCTGTTATATATTGGCCCCACCAAGGTGATA | |
| | CATCACTCGCAAAAGAAATTATGCGACATGCGGATGTTAT | |
| | TGTCGCTTGGGGAGGGCCAGATGCGATTAATTGGGCGGTA | |
| | GAGCATGCGCCATCTTATGCTGATGTGATTAAATTTGGTTC | |
| | TAAAAAGAGTCTTTGCATTATCGATAATCCTGTTGATTTGA | |
| | CGTCCGCAGCGACAGGTGCGGCTCATGATGTTTGTTTTTAC | |
| | GATCAGCGAGCTTGTTTTTCTGCCCAAAACATATATTACAT | |
| | GGGAAATCATTATGAGGAATTTAAGTTAGCGTTGATAGAA | |
| | AAACTTAATCTATATGCGCATATATTACCGAATGCCAAAA | |
| | AAGATTTTGATGAAAAGGCGGCCTATTCTTTAGTTCAAAA | |
| | AGAAAGCTTGTTTGCTGGATTAAAAGTAGAGGTGGATATT | |
| | CATCAACGTTGGATGATTATTGAGTCAAATGCAGGTGTGG | |
| | AATTTAATCAACCACTTGGCAGATGTGTGTACCTTCATCAC | |
| | GTCGATAATATTGAGCAAATATTGCCTTATGTTCAAAAAA | |
| | ATAAGACGCAAACCATATCTATTTTTCCTTGGGAGTCATCA | |
| | TTTAAATATCGAGATGCGTTAGCATTAAAAGGTGCGGAAA | |
| | GGATTGTAGAAGCAGGAATGAATAACATATTTCGAGTTGG | |
| | TGGATCTCATGACGGAATGCGACCGTTGCAACGATTAGTG | |
| | ACATATATTTCTCATGAAAGGCCATCTAACTATACGGCTA | |
| | AGGATGTTGCGGTTGAAATAGAACAGACTCGATTCCTGGA | |
| | AGAAGATAAGTTCCTTGTATTTGTCCCATAATAGGTAAAA | |
| | GTATGGAAAATGAATCAAAATATAAAACCATCGACCACGT | |
| | TATTTGTGTTGAAGGAAATAAAAAAATTCATGTTTGGGAA | |
| | ACGCTGCCAGAAGAAAACAGCCCAAAGAGAAAGAATGCC | |
| | ATTATTATTGCGTCTGGTTTTGCCCGCAGGATGGATCATTT | |
| | TGCTGGTCTGGCGGAATATTTATCGCGGAATGGATTTCAT | |
| | GTGATCCGCTATGATTCGCTTCACCACGTTGGATTGAGTTC | |
| | AGGGACAATTGATGAATTTACAATGTCTATAGGAAAGCAG | |
| | AGCTTGTTAGCAGTGGTTGATTGGTTAACTACACGAAAAA | |
| | TAAATAACTTCGGTATGTTGGCTTCAAGCTTATCTGCGCGG | |
| | ATAGCTTATGCAAGCCTATCTGAAATCAATGCTTCGTTTTT | |
| | AATCACCGCAGTCGGTGTTGTTAACTTAAGATATTCTCTTG | |
| | AAAGAGCTTTAGGGTTTGATTATCTCAGTCTACCCATTAAT | |
| | GAATTGCCGGATAATCTAGATTTTGAAGGCCATAAATTGG | |
| | GTGCTGAAGTCTTTGCGAGAGATTGTCTTGATTTTGGTTGG | |
| | GAAGATTTAGCTTCTACAATTAATAACATGATGTATCTTGA | |
| | TATACCGTTTATTGCTTTTACTGCAAATAACGATAATTGGG | |
| | TCAAGCAAGATGAAGTTATCACATTGTTATCAAATATTCG | |
| | TAGTAATCGATGCAAGATATATTCTTTGTTAGGAAGTTCGC | |
| | ATGACTTGAGTGAAAATTTAGTGGTCCTGCGCAATTTTTAT | |
| | CAATCGGTTACGAAAGCCGCTATCGCGATGGATAATGATC | |
| | ATCTGGATATTGATGTTGATATTACTGAACCGTCATTTGAA | |
| | CATTTAACTATTGCGACAGTCAATGAACGCCGAATGAGAA | |
| | TTGAGATTGAAAATCAAGCAATTTCTCTGTCTTAAAATCTA | |
| | TTGAGATATTCTATCACTCAAATAGCAATATAAGGACTCT | |
| | CTATGAAATTTGGAAACTTTTTGCTTACATACCAACCTCCC | |
| | CAATTTTCTCAAACAGAGGTAATGAAACGTTTGGTTAAAT | |
| | TAGGTCGCATCTCTGAGGAGTGTGGTTTTGATACCGTATG | |
| | GTTACTGGAGCATCATTTCACGGAGTTTGGTTTGCTTGGTA | |
| | ACCCTTATGTCGCTGCTGCATATTTACTTGGCGCGACTAAA | |
| | AAATTGAATGTAGGAACTGCCGCTATTGTTCTTCCCACAG | |
| | CCCATCCAGTACGCCAACTTGAAGATGTGAATTTATTGGA | |
| | TCAAATGTCAAAAGGACGATTTCGGTTTGGTATTTGCCGA | |
| | GGGCTTTACAACAAGGACTTTCGCGTATTCGGCACAGATA | |
| | TGAATAACAGTCGCGCCTTAGCGGAATGCTGGTACGGGCT | |
| | GATAAAGAATGGCATGACAGAGGGATATATGGAAGCTGA | |
| | TAATGAACATATCAAGTTCCATAAGGTAAAAGTAAACCCC | |
| | GCGGCGTATAGCAGAGGTGGCGCACCGGTTTATGTGGTGG | |
| | CTGAATCAGCTTCGACGACTGAGTGGGCTGCTCAATTTGG | |
| | CCTACCGATGATATTAAGTTGGATTATAAATACTAACGAA | |
| | AAGAAAGCACAACTTGAGCTTTATAATGAAGTGGCTCAAG | |
| | AATATGGGCACGATATTCATAATATCGACCATTGCTTATC | |
| | ATATATAACATCTGTAGATCATGACTCAATTAAAGCGAAA | |
| | GAGATTTGCCGGAAATTTCTGGGGCATTGGTATGATTCTTA | |
| | TGTGAATGCTACGACTATTTTTGATGATTCAGACCAAACA | |
| | AGAGGTTATGATTTCAATAAAGGGCAGTGGCGTGACTTTG | |
| | TATTAAAAGGACATAAAGATACTAATCGCCGTATTGATTA | |
| | CAGTTACGAAATCAATCCCGTGGGAACGCCGCAGGAATGT | |
| | ATTGACATAATTCAAAAAGACATTGATGCTACAGGAATAT | |
| | CAAATATTTGTTGTGGATTTGAAGCTAATGGAACAGTAGA | |
| | CGAAATTATTGCTTCCATGAAGCTCTTCCAGTCTGATGTCA | |
| | TGCCATTTCTTAAAGAAAAACAACGTTCGCTATTATATTAG | |
| | CTAAGGAGAAAGAAATGAAATTTGGATTGTTCTTCCTTAA | |
| | CTTCATCAATTCAACAACTGTTCAAGAACAAAGTATAGTT | |
| | CGCATGCAGGAAATAACGGAGTATGTTGATAAGTTGAATT | |
| | TTGAACAGATTTTAGTGTATGAAAATCATTTTTCAGATAAT | |
| | GGTGTTGTCGGCGCTCCTCTGACTGTTTCTGGTTTTCTGCT | |
| | CGGTTTAACAGAGAAAATTAAAATTGGTTCATTAAATCAC | |
| | ATCATTACAACTCATCATCCTGTCGCCATAGCGGAGGAAG | |
| | CTTGCTTATTGGATCAGTTAAGTGAAGGGAGATTTATTTTA | |
| | GGGTTTAGTGATTGCGAAAAAAAAGATGAAATGCATTTTT | |
| | TTAATCGCCCGGTTGAATATCAACAGCAACTATTTGAAGA | |
| | GTGTTATGAAATCATTAACGATGCTTTAACAACAGGCTAT | |
| | TGTAATCCAGATAACGATTTTTATAGCTTCCCTAAAATATC | |
| | TGTAAATCCCCATGCTTATACGCCAGGCGGACCTCGGAAA | |
| | TATGTAACAGCAACCAGTCATCATATTGTTGAGTGGGCGG | |
| | CCAAAAAAGGTATTCCTCTCATCTTTAAGTGGGATGATTCT | |
| | AATGATGTTAGATATGAATATGCTGAAAGATATAAAGCCG | |
| | TTGCGGATAAATATGACGTTGACCTATCAGAGATAGACCA | |
| | TCAGTTAATGATATTAGTTAACTATAACGAAGATAGTAAT | |
| | AAAGCTAAACAAGAGACGCGTGCATTTATTAGTGATTATG | |
| | TTCTTGAAATGCACCCTAATGAAAATTTCGAAAATAAACT | |
| | TGAAGAAATAATTGCAGAAAACGCTGTCGGAAATTATACG | |
| | GAGTGTATAACTGCGGCTAAGTTGGCAATTGAAAAGTGTG | |
| | GTGCGAAAAGTGTATTGCTGTCCTTTGAACCAATGAATGA | |
| | TTTGATGAGCCAAAAAAATGTAATCAATATTGTTGATGAT | |
| | AATATTAAGAAGTACCACATGGAATATACCTAATAGATTT | |
| | CGAGTTGCAGCGAGGCGGCAAGTGAACGAATCCCCAGGA | |
| | GCATAGATAACTATGTGACTGGGGTGAGTGAAAGCAGCCA | |
| | ACAAAGCAGCAGCTTGAAAGATGAAGGGTATAAAAGAGT | |
| | ATGACAGCAGTGCTGCCATACTTTCTAATATTATCTTGAGG | |
| | AGTAAAACAGGTATGACTTCATATGTTGATAAACAAGAAA | |
| | TTACAGCAAGCTCAGAAATTGATGATTTGATTTTTTCGAGC | |
| | GATCCATTAGTGTGGTCTTACGACGAGCAGGAAAAAATCA | |
| | GAAAGAAACTTGTGCTTGATGCATTTCGTAATCATTATAA | |
| | ACATTGTCGAGAATATCGTCACTACTGTCAGGCACACAAA | |
| | GTAGATGACAATATTACGGAAATTGATGACATACCTGTAT | |
| | TCCCAACATCGGTTTTTAAGTTTACTCGCTTATTAACTTCT | |
| | CAGGAAAACGAGATTGAAAGTTGGTTTACCAGTAGCGGCA | |
| | CGAATGGTTTAAAAAGTCAGGTGGCGCGTGACAGATTAAG | |
| | TATTGAGAGACTCTTAGGCTCTGTGAGTTATGGCATGAAA | |
| | TATGTTGGTAGTTGGTTTGATCATCAAATAGAATTAGTCAA | |
| | TTTGGGACCAGATAGATTTAATGCTCATAATATTTGGTTTA | |
| | AATATGTTATGAGTTTGGTGGAATTGTTATATCCTACGACA | |
| | TTTACCGTAACAGAAGAACGAATAGATTTTGTTAAAACAT | |
| | TGAATAGTCTTGAACGAATAAAAAATCAAGGGAAAGATCT | |
| | TTGTCTTATTGGTTCGCCATACTTTATTTATTTACTCTGCCA | |
| | TTATATGAAAGATAAAAAAATCTCATTTTCTGGAGATAAA | |
| | AGCCTTTATATCATAACCGGAGGCGGCTGGAAAAGTTACG | |
| | AAAAAGAATCTCTGAAACGTGATGATTTCAATCATCTTTT | |
| | ATTTGATACTTTCAATCTCAGTGATATTAGTCAGATCCGAG | |
| | ATATATTTAATCAAGTTGAACTCAACACTTGTTTCTTTGAG | |
| | GATGAAATGCAGCGTAAACATGTTCCGCCGTGGGTATATG | |
| | CGCGAGCGCTTGATCCTGAAACGTTGAAACCTGTACCTGA | |
| | TGGAACGCCGGGGTTGATGAGTTATATGGATGCGTCAGCA | |
| | ACCAGTTATCCAGCATTTATTGTTACCGATGATGTCGGGAT | |
| | AATTAGCAGAGAATATGGTAAGTATCCCGGCGTGCTCGTT | |
| | GAAATTTTACGTCGCGTCAATACGAGGACGCAGAAAGGGT | |
| | GTGCTTTAAGCTTAACCGAAGCGTTTGATAGTTGA | |
| RBS30 | ATTAAAGAGGAGAAA | 11 |
| RBS35 | ATTAAAGAGGAGAA | 12 |
| RBS64 | AAAGAGGGGAAA | 13 |
| Bxb1B | CGGCCGGCTTGTCGACGACGGCGGTCTCCGTCGTCAGGAT | 14 |
| | CATCCGGGC | |
| Bxb1P | GTCGTGGTTTGTCTGGTCAACCACCGCGGTCTCAGTGGTGT | 15 |
| | ACGGTACAAACCCCGAC | |
| PnorV | ACGGAAAAACTCATCTTTGCCTCACTGTCAATTTGACTATA | 16 |
| | GATATTGTCATATCGACCATTTGATTGATAGTCATTTTGAC | |
| | TACTCATTAATGGGCATAATTTTATTTATAGAGTAAAAAC | |
| | AATCAGATAAAAAACTGGCACGCAATCTGCAATTAGCAAG | |
| | ACATCTTTTTAGAACACG | |
| NorR | ATGAGTTTTTCCGTTGATGTGCTGGCGAATATCGCCATCGA | 17 |
| | ATTGCAGCGTGGGATTGGTCACCAGGATCGTTTTCAGCGC | |
| | CTGATCACCACGCTACGTCAGGTGCTGGAGTGCGATGCGT | |
| | CTGCGTTGCTACGTTACGATTCGCGGCAGTTTATTCCGCTT | |
| | GCCATCGACGGTCTGGCAAAGGATGTACTCGGTAGACGCT | |
| | TTGCGCTGGAAGGGCATCCACGGCTGGAAGCGATTGCCCG | |
| | CGCCGGGGATGTGGTGCGCTTTCCCGCAGACAGCGAATTG | |
| | CCCGATCCCTATGACGGTTTGATTCCTGGGCAGGAGAGTC | |
| | TGAAGGTTCACGCCTGCGTTGGTCTGCCATTGTTTGCCGGG | |
| | CAAAACCTGATCGGCGCACTGACGCTCGACGGGATGCAGC | |
| | CCGATCAGTTCGATGTTTTCAGCGACGAAGAGCTACGGCT | |
| | GATTGCTGCGCTGGCGGCGGGAGCGTTAAGCAATGCGTTG | |
| | CTGATTGAACAACTGGAAAGCCAGAATATGCTGCCAGGCG | |
| | ATGCCACGCCGTTTGAAGCGGTGAAACAGACGCAGATGAT | |
| | TGGCTTGTCCCCTGGCATGACGCAACTGAAAAAAGAGATT | |
| | GAGATTGTGGCGGCGTCCGATCTCAACGTCCTGATCAGCG | |
| | GTGAGACTGGAACCGGTAAGGAGCTGGTGGCGAAAGCGA | |
| | TTCATGAAGCCTCGCCACGGGCGGTGAATCCGCTGGTCTA | |
| | TCTCAACTGTGCTGCACTGCTGGAAAGTGTGGCGGAAAGT | |
| | GAGTTGTTCGGGCATGTGAAAGGAGCGTATACTGGCGCTA | |
| | TCAGTAATCGCAGCGGGAAGTTTGAAATGGCGGATAACGG | |
| | CACGCTGTTTCTGGATGAGATCGGCGAGTTGTCGTTGGCA | |
| | TCGCAGGCCAAGCTGCTGAGGGTGTTGCAGTATGGCGATA | |
| | TTCAGCGCGTTGGCGATGACCGTTGTTTGCGGGTCGATGT | |
| | GCGCGTGCTGGCGGCGACTAACCGCGATTTACGCGAAGAG | |
| | GTGCTGGCAGGGCGATTCCGCGCCGATTTGTTTCATCGCCT | |
| | GAGCGTGTTTCCACTTTCGGTGCCGCCGCTGCGTGAGCGG | |
| | GGCGATGATGTCATTCTGCTGGCGGGGTATTTCTGCGAGC | |
| | AGTGTCGTTTGCGGCAGGGGCTCTCCCGCGTGGTATTAAG | |
| | TGCCGGAGCGCGAAATTTACTGCAACACTACAGTTTTCCG | |
| | GGAAACGTGCGCGAACTGGAACATGCTATTCATCGGGCGG | |
| | TAGTTCTGGCGAGAGCCACCCGCAGCGGCGATGAAGTGAT | |
| | TCTTGAGGCGCAACATTTTGCGCGCGCGGATGCTGGAAAC | |
| | CGACGTCGCCAACCTGCATCGGCTGGCGAAACGCGCGCGG | |
| | ATGCTGGAAACGCGACAGAAGCGTTCCAGCGTGAAACTAT | |
| | TCGTCAGGCACTGGCACAAAATCATCACAACTGGGCTGCC | |
| | TGCGCGCGGATGCTGGAAACCGACGTCGCCAACCTGCATC | |
| | GGCTGGCGAAACGCGCGCGGATGCTGGAAACCGACGTCG | |
| | CCAACCTGCATCGGCTGGCGAAACGTCTGGGATTGAAGGA | |
| | TTAA | |
| TtrR | ATGAGCCTGGCGCTGCCGGTGTACCTGATTGACGACGATG | 18 |
| | ACTCCGTTCGCCGTTCCCTGCGTTTCATGCTGGAAAGCTAC | |
| | GGCCTGAAAATCACTGACTTCGATTCTGCTGAAGCGTTCTT | |
| | CACCGCGGTAGACCTGACCCTGCCGGGTTGCGCACTGGTG | |
| | GACGTACGTATGCCGGGCCTGAGCGGCCCACAGCTGCACC | |
| | TGGAACTGGTGTCTAAGAACAGCCCGCTGGCCGTGATCTA | |
| | TCTGACCGGTCACGGCGACGTTCCGATGGCGGTTGAAGCG | |
| | CTGAAACTGGGTGCGGTAGATTTCTTTCAGAAACCTGCAG | |
| | ACGGCGCTAAACTGGCTGAAGCTGTGGTCAAAGCCCTGGA | |
| | ACACGCGAAAACCCACTACCAAGACAACCAGTACCTGGA | |
| | AACCTATCAGGCTCTGACCCCACGTGAGCGCGAAATCCTG | |
| | AACCTGATTGCGCAAGGTCTGAAAAACCAGGAAATCGCG | |
| | GACAACCTGTGCATTGCGATGCGCACCGTAGAAATCCACC | |
| | GTGCGAACCTGATGAAAGGTATGCAGGTGGGCAGCCTGGC | |
| | AGAGATGATGCTGATTTACTCTCGTATCGCGGAGCGTCTG | |
| | CGCCTGGAAGATAACCGTTAA | |
| TtrS | ATGTTTTCTTCCGGCATGAGCCTGAAATGGCCTCTGAGCAT | 19 |
| | CCAGGGTTCCATCGAACCGAAAATGACTCCGCGCATCATC | |
| | TTCCGCATGGCCCTGCGCACTAAAGTGTTCATCACCCTGCT | |
| | GGCTTGCCTGTTTGCTTCTGCCAACGTATTCGCGGTGGAAC | |
| | CGCAGAACCAGTCCGCGCCTTCCGATGTGATGGACGCTGA | |
| | GGTGGTTGCTCCGGTTGCGACGGAGGAACCGGGTTACCAG | |
| | GTCGTTGATGTTGGTGTGCTGGCGATTCGCGGTTATAAAG | |
| | CTACCATCAACCGTTGGCAGCCACTGATGGGTTGGCTGGA | |
| | GACTCAGATCCCGAACTCCTACTTCCGCCTGCACCCGCTGT | |
| | CTCTGGACGAGCTGGCAAAGGGTGTAGAAACCCAGGGTCT | |
| | GGACTTCGTAATCACCAATCCAGGCCAGTCCGTACTGCTG | |
| | GCGCGTCAGTATTCTCTGACGTGGCTGGCGACCCTGCGCT | |
| | CTCCGCTGAACAACGGTGCTGCGATGCAGGTGGGTTCCGC | |
| | GCTGGTAGTTCGTGCCGACAGCCCGTACCAGACCCTGACC | |
| | GATCTGAAAGGCCGTCGTCTGGGCATCGTCAGCAAAAACG | |
| | CATTCGGTGGTTATCTGACGCTGGTATATGAGGCACAGCT | |
| | GAAAGGCATCGATCTGCCGCGCTTTGTTGGTGAAATCATC | |
| | CCACTGGGCTTCCCGCTGGATAACCTGCTGTATCAGCTGG | |
| | ATGATCAGAAATCTGGTAATGACGCCGCAAAAGACAATCG | |
| | TCTGGATGCCACCGTTGTACCGGTATGCCAACTGGAACAG | |
| | ATGCAGGCCGAAGGTCTGATCAACATCGGCCACTATCGCG | |
| | TTCTGGACAACCAGACCCCGGTAGGTTTCCACTGTCAAGT | |
| | AAGCACTCGTCTGTATCCGAACTGGTCTATGGCTAAAACT | |
| | AACCGTGCCAGCCAGTCCCTGGCAAAATCTGTGACCCAGG | |
| | CTCTGCTGGCTCTGCCGGAAGATCACCTGGCAGCGAAAAG | |
| | CTCTGACTCTGCAGGTTGGACCACTGCAGTGTCCCAGCTG | |
| | GCGATCGACCAGCTGCTGAAAGACCTGGACATGCATCCAC | |
| | TGCAGACCCCATGGTGGCAGCGCGCTTGGCAGTGGGTAAA | |
| | ACTGCACCAGCAGTGGGCGTGGTTCATTCTGGGTATCCTG | |
| | GTCCTGCTGAACGCATACCATTTCTGGCTGGAATACCGTTT | |
| | CTCCCGTCGTGGTCGTGAACTGATCAACACCCAGCGCCAG | |
| | CTGAACGAAAACCGTGCTCTGCTGGAGCACGCACAACGTA | |
| | TCGCCATCGCTGGCGAACTGGGTGCTAGCCTGTCCCATGA | |
| | ACTGAACCAGCCGCTGGCTGCAATTGGTCACTATTGCCAT | |
| | GGTGCGGAAGTGCGTCTGCAGCGTGGTACTTCTCCTGAAG | |
| | AACTGCAGTCTGTTCTGACCCTGATCCAGCAGGAAGTTAC | |
| | GCGCGCCGACTCTATCATCAGCCGTCTGCGTAACCTGCTG | |
| | AAAAAACGCCCGGTTAGCAAACAGGCTCTGTATCTGCACG | |
| | AACTGGTAAACGAAACGGTGCCGCTGCTGGCGTACGAATT | |
| | CGAACAGCACCAGATCAACCTGGCGGTAAACGTCAGCGGT | |
| | GAACCTTACCTGCAATCTCTGGACGAGGTCGGTATGCAGC | |
| | AGCTGCTGCTGAACCTGCTGAAAAACGCGTTTGACGCGTG | |
| | CGTTCAGCGCCTGGAACTGGAATCCTCCGGCACTGAACAG | |
| | AGCGGTATGCGTAAACCGTATGTTCCTACGATCGATATTG | |
| | ATCTGCGCTACCAGGAGCGTACTCTGCTGCTGACTGTGAC | |
| | GGATAACGGCACTGGTCTGACCGAGGAGACTAGCCTGCTG | |
| | ATGCAGGCCTTCTATTCCACCAAATCTGAAGGCCTGGGCC | |
| | TGGGTCTGGTTATTTGCCGTGATATCGCTGAGTCCCACGGT | |
| | GGCACCTTCTCTCTGGAATCCGCCATGGGTGGTGGCTGCC | |
| | AGGCTCAGGTGGCCATCCCGCGCAAGCCGGAACCGAATG | |
| | GCGTTCTGTAA | |
| TtrB | TTTATAGTAAATCACTGCATAATTCGTGTCGCTCAAGGCGC | 20 |
| | ACTCCCGTTCTGGATAATGTTTTTTGCGCCGACATCATAAC | |
| | GGTTCTGGCAAATATTCTGAAATGAGCTGGTTAGCTAGTC | |
| | AATATTTGTTGCGCTAGATCAAATCCACGCCTGATATGTG | |
| | GAAAACCACTATAGTTATGCCGCCTCGCCTTTTACAGAAT | |
| | GCCTGT | |

### Example 3

The following example describes the design and fabrication of several biosensors configured to detect one or more biomarkers.

Inflammatory bowel disease (IBD) is an intestinal disorder hallmarked by chronic inflammation, which may be caused as a consequence of a dysregulated intestinal immune homeostasis. Although its etiology is not well defined, a perturbed chemical landscape with increased oxidation has been associated with promoting IBD onset. Bacterial infections and antibiotics may increase the concentrations of high-potential terminal electron acceptors (oxidants) such as reactive oxygen and reactive nitrogen species (ROS/RNS) but there are, so far, no effective ways to track these processes since these molecules that mediate disease are labile. Other microbiome-related conditions associated with redox imbalances in the gut may include malnutrition, antibiotic resistance, antibiotic dysbiosis, neurodegenerative diseases, and mitochondrial genetic disorders (e.g., diabetes mellitus, autism, and developmental delay). Poor response to chemotherapy and vaccines, as well as aging, may also reflect a redox imbalance. Because the chemical environment determines the metabolic pathways that can occur both in the host and in resident microbes, without non-invasive technologies to monitor this environment in situ and over time, the understanding of what causes inflammation and diagnostic and therapeutic capacity are limited.

Current methods of diagnosing GI inflammation include endoscopy, which is invasive and limited by the frequency that can safely be performed, and laboratory analyses of stool, which may result in differential growth of certain species, ambient oxidation, or loss of labile disease-mediating molecules. Because the fecal microbiota only partially represents the autochthonous microbiota in direct contact with the intestinal mucosa, biopsy may be required. Culture enrichment (e.g., to analyze stool samples with “omics” techniques) could also distort the initial bacterial ratio.

Electronic devices may efficiently process and wirelessly transmit information and continuously monitor disease. However, capsule endoscopes approved by the US Food and Drug Administration (FDA) do not yet directly measure the molecular mediators of disease. Ingestible electronic devices designed with ultra-low power microelectronics can visually evaluate the GI tract and measure gases, temperature, and pH but require functionalization with transducers that convert biochemical information into electronic signals, limiting specificity and robustness.

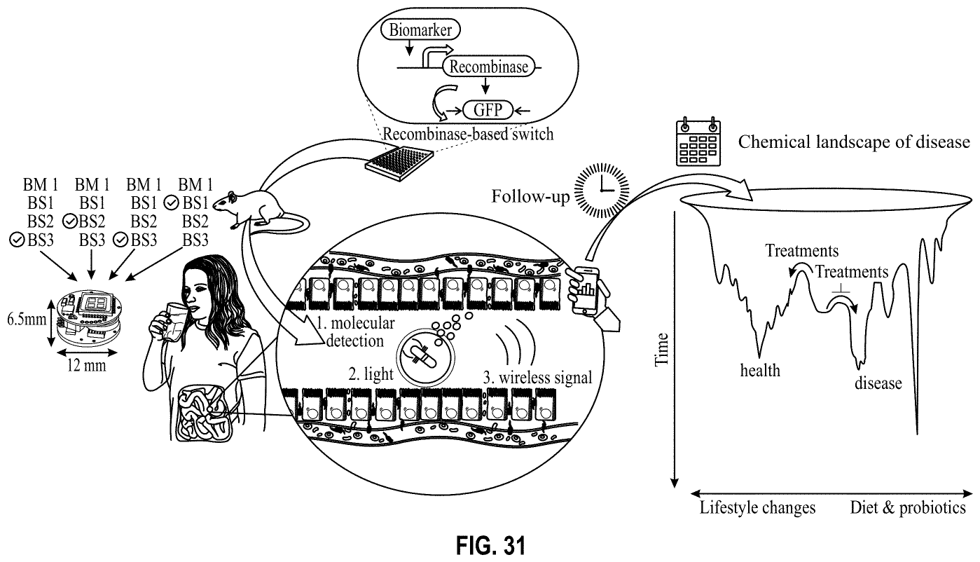

In this example, natural sensors were incorporated in intestinal bacteria to record the transient presence of host by-products of inflammation (i.e., NO and ROS), intestinal gases (i.e., H₂S converted to thiosulfate) and microbiota-related biomolecules (i.e., tetrathionate). These engineered bacteria can detect metabolic by-products, and microbiota-related molecules in an inflammation model in rodents. Profiling these molecules and their responses to dietary change and therapeutics enhanced the ability to diagnose and monitor inflammatory GI disorders. A miniaturized wireless bio-electronic system was developed that can safely transmit data to a wearable or portable device, e.g., a smartphone. By integrating the diagnostic pill in a model of inflammation in pigs, this example helped validated the technologies include to make a human-scale diagnostic device for mediators of GI inflammatory disease (FIG. 31).

Natural sensors were incorporated in intestinal bacteria to record the transient presence of host by-products of inflammation (e.g., NO and ROS), intestinal gases (e.g., H₂S converted to thiosulfate) and microbiota-related biomolecules (i.e., tetrathionate). The engineered bacteria were able to detect metabolic byproducts, and microbiota-related molecules in an inflammation model in rodents. It was discovered that profiling these molecules and their responses to dietary change and therapeutics enhanced the ability to diagnose and monitor inflammatory GI disorders. In some case, a miniaturized wireless bio-electronic system that can safely transmit data to a wearable or portable device may be fabricated, e.g., a smartphone. By integrating the diagnostic pill in a model of inflammation in pigs, this example validated the technologies required to make a human-scale diagnostic device for mediators of GI inflammatory disease (FIG. 31).

### Results

### Design of Biosensing Genetic Circuits

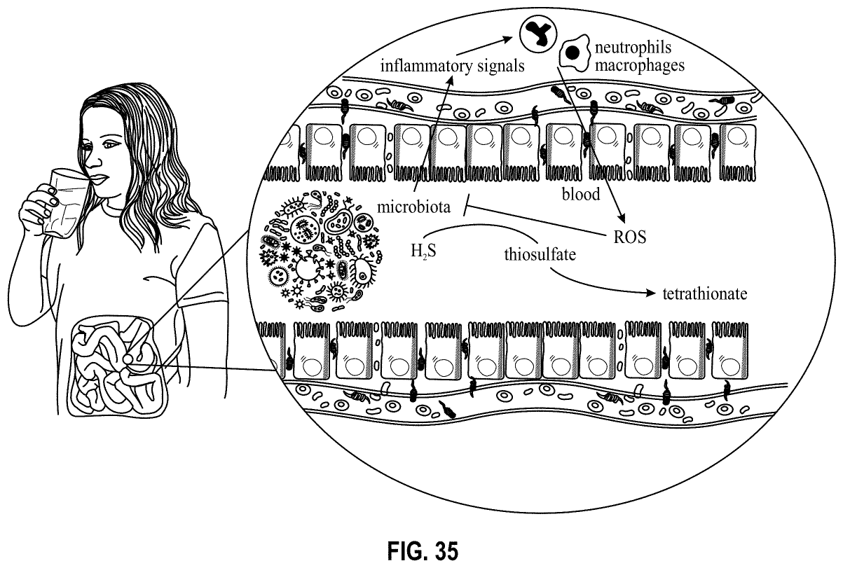

Intestinal bacteria are equipped with natural sensors that continuously sense specific molecules in their environment. These bacteria may be resilient in the GI tract, which may be a relatively harsh and difficult-to-access environment. Considering the potential immunogenic response to diagnostic microbes, probiotic E. coli Nissle (EcN) was chosen as a chassis for its excellent safety profile for long-term use and engineered it to specifically detect the labile IBD-mediating molecules nitric oxide (NO), peroxide (H₂O₂), thiosulfate and tetrathionate (FIG. 35).

For the in vivo validation of the diagnostic sensors in inflammation models in rodents, memory circuits were fabricated to report the exposure to NO when bacterial biosensors are recovered in feces. Several bacterial NO sensors control the expression of cognate NO reductases to detoxify NO inside the gut. NorR was chosen because it differentiates NO from other reactive nitrogen species (NOx) rich in the gut environment. NorR activates transcription from the norV promoter (PnorV).

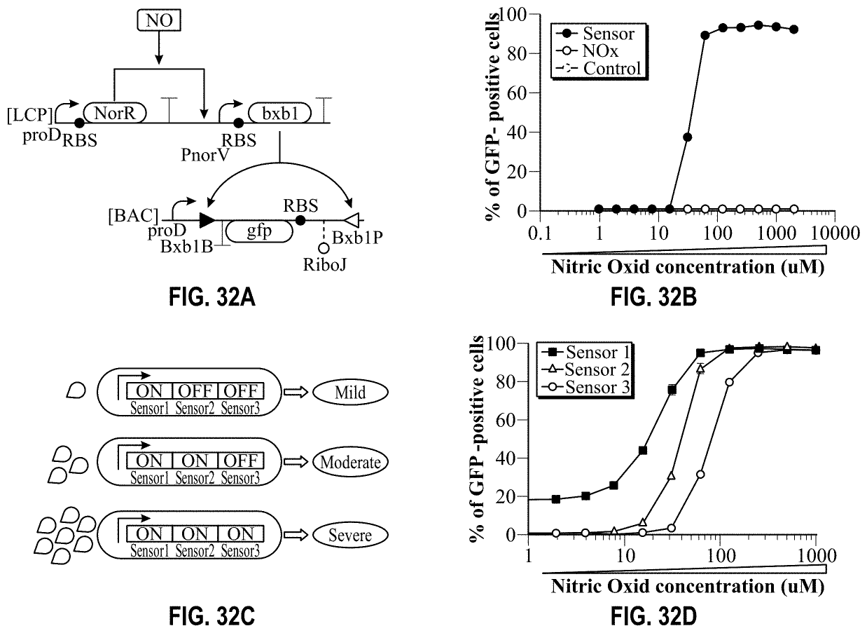

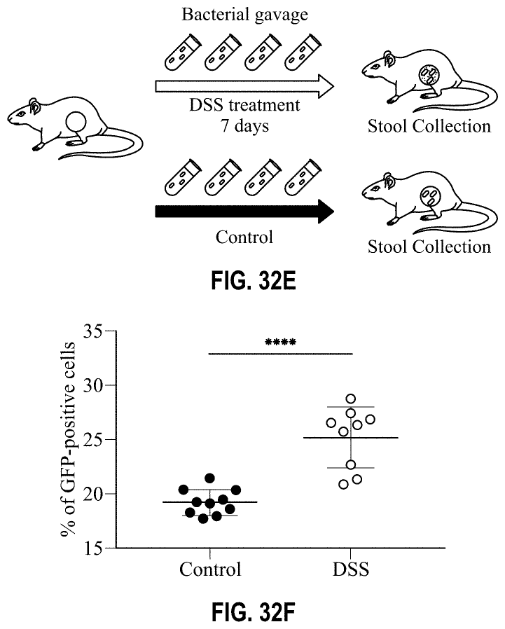

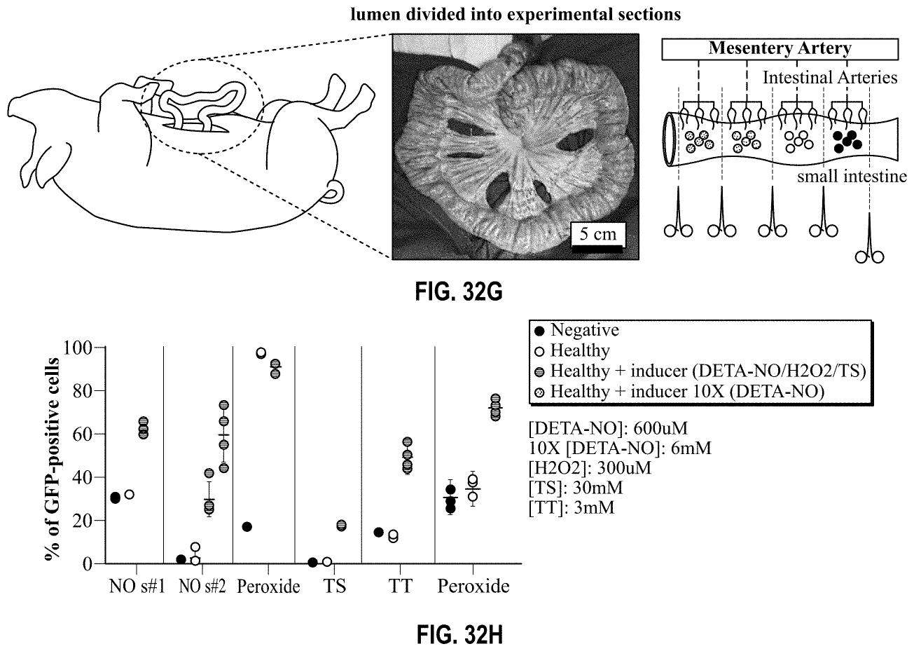

Recombinases recognize specific DNA sequences and can invert them, leaving long-lasting changes in DNA. To create memory circuits to report exposure to NO, NO sensing was combined with a DNA recombinase core circuit by placing recombinase bxb1 transcription under the control of the PnorV promoter on a low copy number plasmid (FIG. 32A). A bacterial artificial chromosome (BAC) was used, which contained a constitutive promoter upstream of an inverted gfp gene flanked by recombinase-recognition sites. When activated by the biomarker, the Bxb1 serine recombinase flips the stretch of DNA in between those sites, and GFP is expressed. Once an exposure is recorded through this process, the information is stored in the DNA of the bacteria, passed from generation to generation, and retrievable directly by measuring GFP expression.

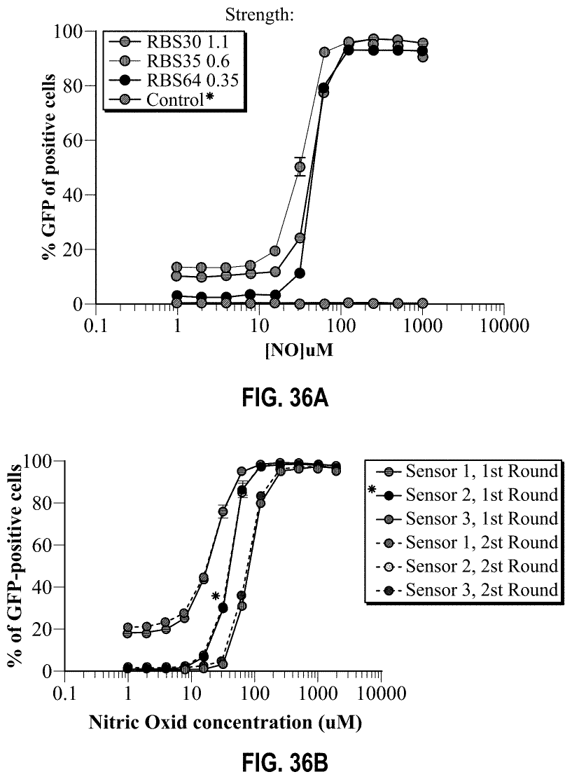

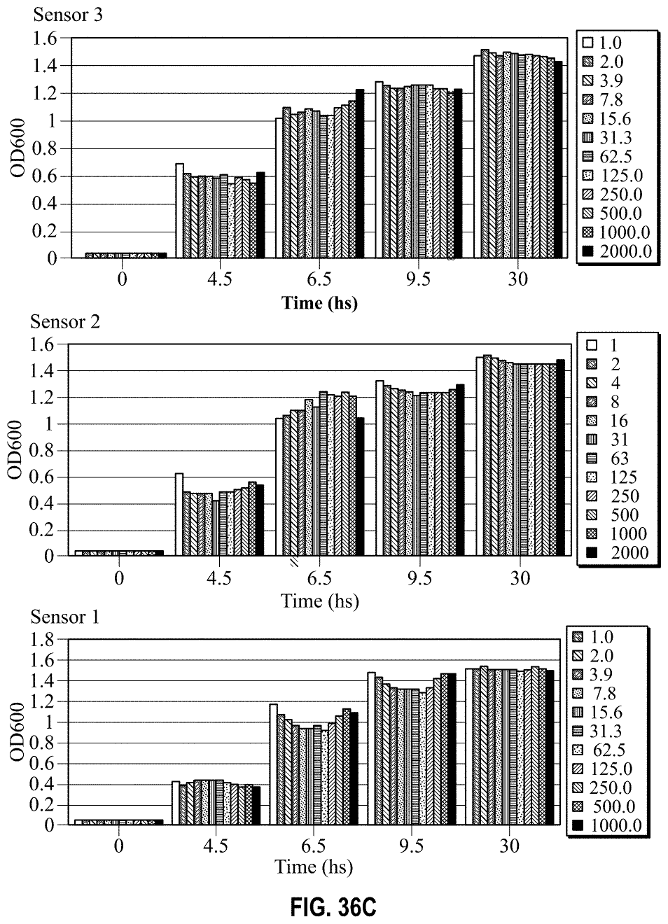

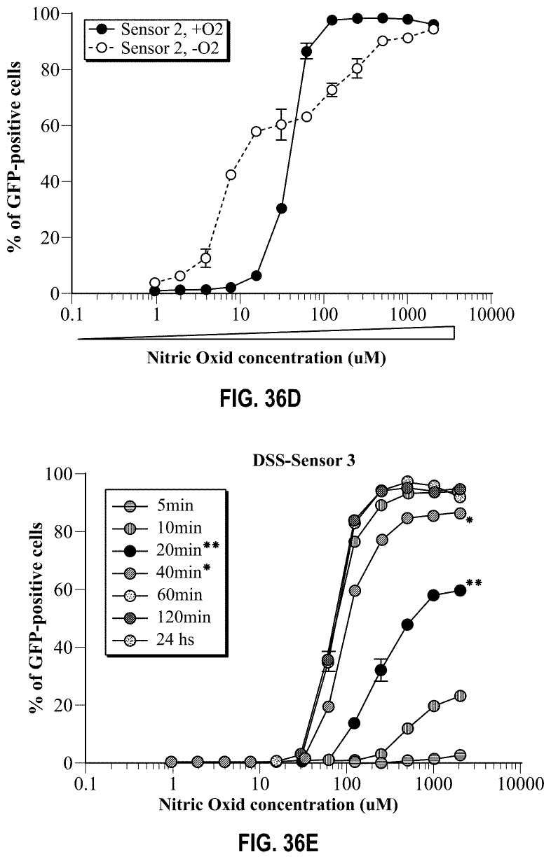

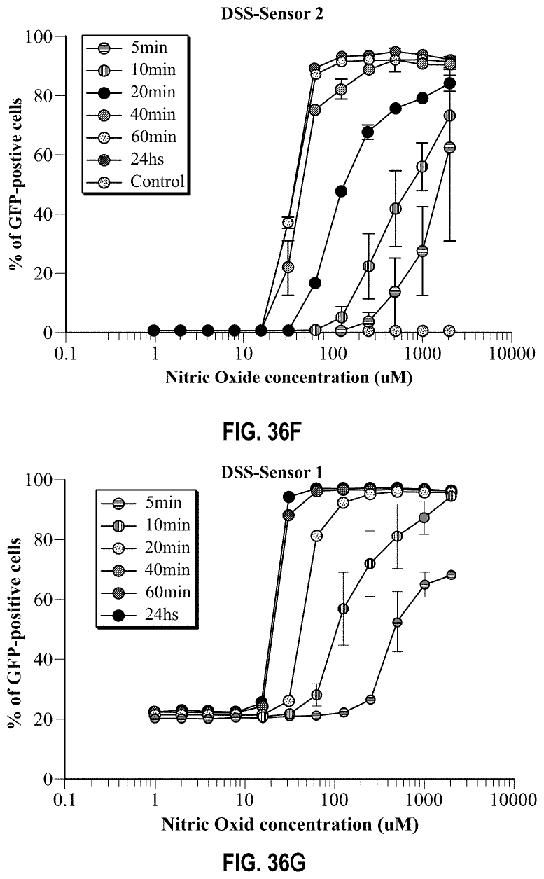

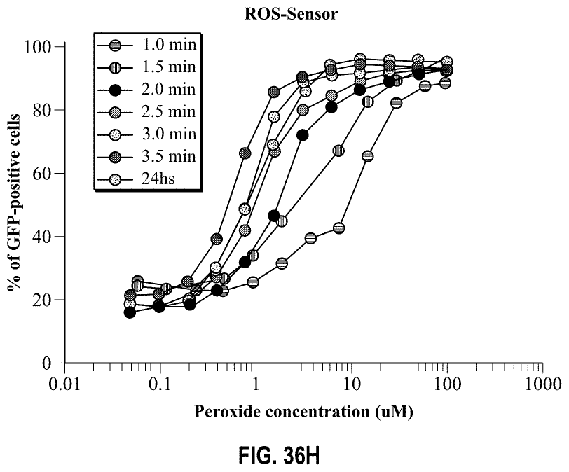

GFP expression were measured via flow cytometry as a function of the concentration of DETA/NO (diethylenetriamine/nitric oxide adduct). A threshold for calling cells GFP ‘ON’ or ‘OFF’ was set and used to calculate the percent of cells that were ON (% ON) at each concentration of NO. After multiple genetic circuit optimizations (FIG. 36A) and controls (FIGS. 36B-36C), the NO Recombinase-based Memory System responded to a concentration threshold of 30 μM diethylenetriamine/nitric oxide (DETA/NO) and operated orthogonally with respect to NOx compounds that could be found in the diet, showing the high specificity for NO (FIG. 32B). The NO sensor performance was also validated under anaerobic conditions (FIG. 36D).

A disease stage detector (DSD) was also created based on NO detection, in which each state level could be indicative of disease (FIGS. 32C-32D). The incorporated recombinase-based switch was also used to discretize the biomarker input levels and create digital memories in the cells. Tuning the sensitivity of each biosensor resulted in different activation thresholds, so that physiologically relevant concentrations of the biomarker (low, medium, or high) could be measured in different animal models. For the sensors developed for in vivo validation in mice, GFP expression was measured as a readout of the activation of the memory system, activated within minutes (FIGS. 36E-36H).

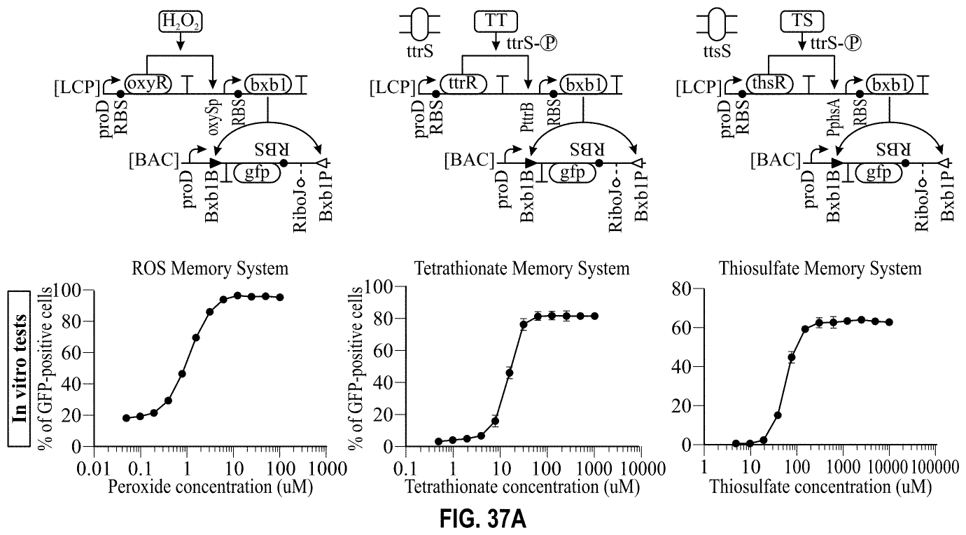

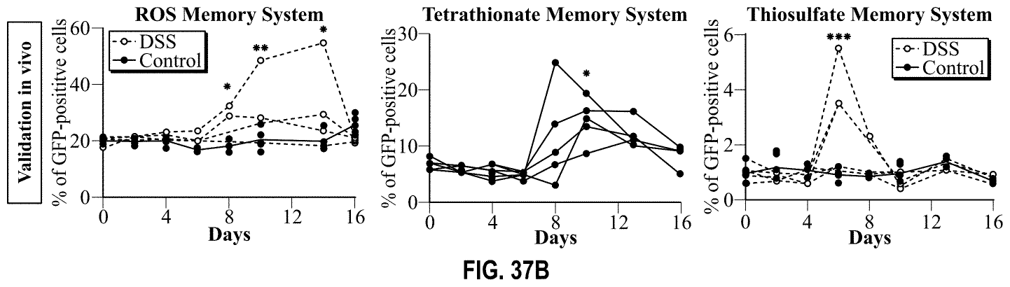

The ROS biosensor detects H₂O₂, which oxidizes and activates the transcription factor OxyR. The recombinase gene bxb1 is under the control of the OxyR-regulated oxyS promoter, oxySp, on the same genetic circuit (FIG. 37A).

The TtrSR two-component system (TCS) from Salmonella typhimurium, which has been used as a genetically encoded sensor for detecting sulfur metabolism in the gut, naturally activates transcription of the tetrathionate reductase operon, ttrBCA, via the ttrB promoter (PttrB). However, the global regulator FNR (Fumarate and Nitrate Reductase Regulator), required for transcription from PttrB, is repressed by oxygen (O₂). Thus, using PttrB as a readout for tetrathionate sensing is potentially confounded by fluctuating O₂ levels due to FNR regulation. This cross-regulation could compromise the performance of TtrSR-based tetrathionate sensing in the gut, where O₂ levels vary depending on their proximity to the epithelial mucosal boundary and fluctuate depending on the level of mucosal disruption.

To avoid this cross-repression, two new identified sensors to express the recombinase system for detecting thiosulfate and tetrathionate (FIG. 37A): (1) a novel tetrathionate sensor from the marine bacterium Shewanella baltica, which bypasses the FNR system, and (2) the ThsRS from Shewanella halifaxensis, the only genetically encoded thiosulfate sensor characterized so far. Both sensors distinguish their target molecules from other terminal electron acceptors in vitro.

### Bacterial Sensors Detect Disease in Animal Models of IBD

To examine the functionality of the engineered inflammatory biosensors in rodent models of IBD in vivo, the bacterial sensors were evaluated to determine if passing through the gut could detect GI inflammation in a chemically induced mouse model of colitis (FIG. 32E-32F). After one week of 3% DSS treatment in drinking water, both control and treated mice were orally gavaged with the NO biosensors and after 6 hrs, fecal samples were collected from both groups to analyze the percentage of GFP+ cells recovered by flow cytometry.

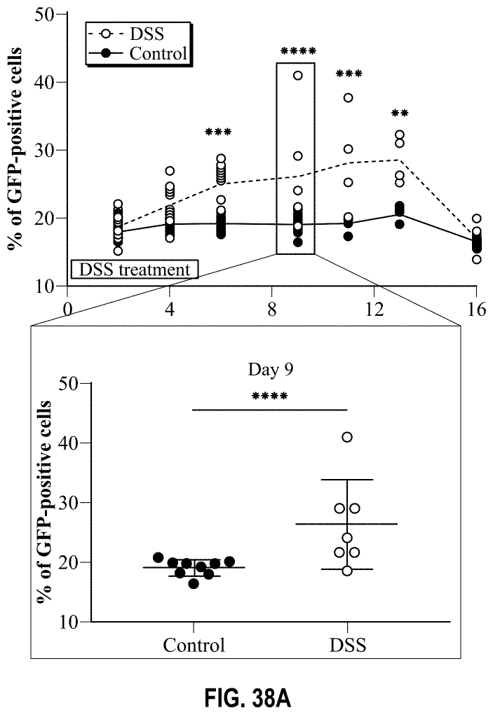

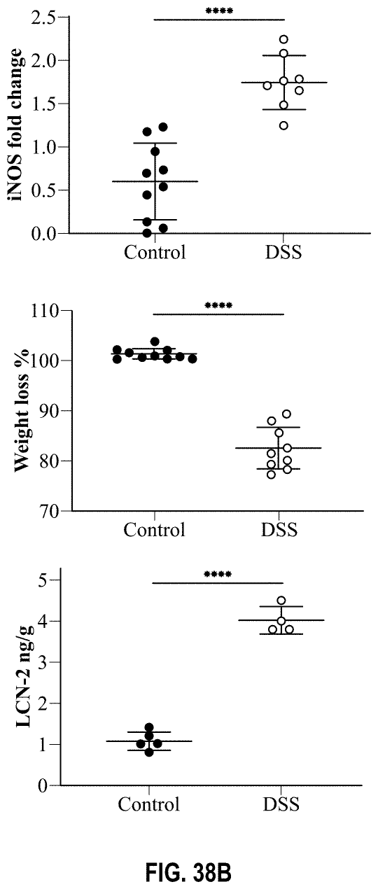

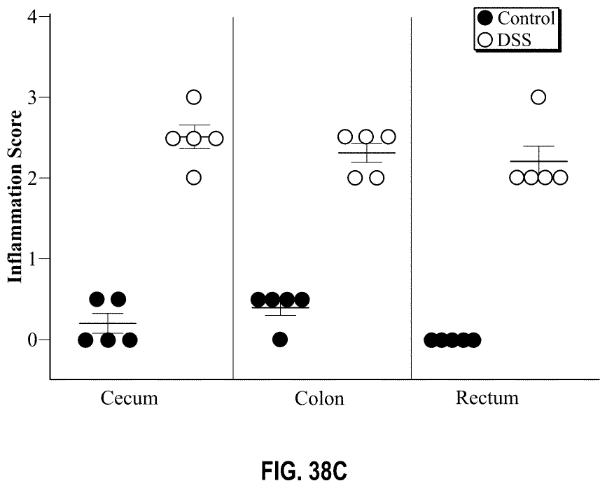

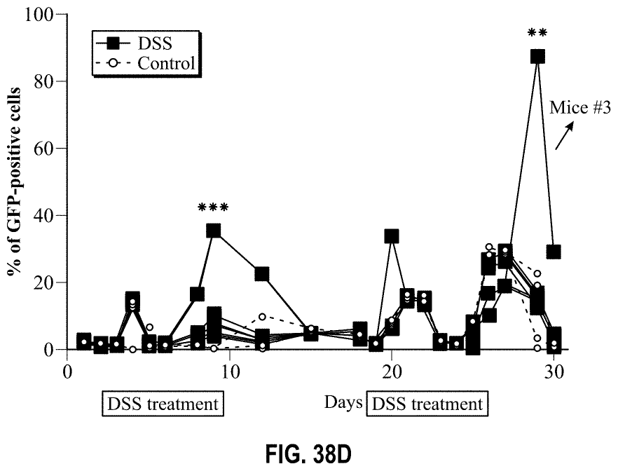

After oral administration of DSS, the inflammation-sensing circuits coupled to recombinase-based memory registered differences between healthy and diseased mice. NO biosensors demonstrated significant GFP+ activation values in fecal pellets as compared to healthy controls (FIG. 32F). The designed biosensor thus detected the presence of NO as a marker of GI inflammation in vivo. Inflammation in the colitis model was independently validated by quantifying the lipocalin-2 (LCN-2) biomarker, iNOS expression, histological score, and other parameters such as weight, bloody and loose stools, poor vigor, anal prolapse, as well as shortening of the colon upon dissection and gross morphological examination (FIG. 38A-S4B).

The significant increase of GFP activation at day 9 after DSS treatment (FIG. 38A) correlated with the peak of iNOS activation. Besides, tracking NO with the DSD allowed for the detection of an exacerbated inflammatory response after an antibiotic treatment in a DSS-chronic inflammation model (FIG. 38D). The bacterial sensors were also tested for ROS (e.g., hydrogen peroxide), tetrathionate, and thiosulfate, following a similar protocol as just described; a similar activation was detected as inflammation progressed (FIG. 37B).

While the biosensor cells could be validated in rodents, validating mm-scale diagnostic devices used a model comparable in scale to human anatomy. Therefore, the bacterial biosensors were also tested in pigs. The biosensors were placed through a small incision directly into the small intestine (jejunum) of the sedated animal, with or without added biomarkers. The NO sensor bacteria produced a signal only in the treated group, as seen in the rodent model, and the [ROS], thiosulfate and tetrathionate sensors also showed significant detection.

### In Vitro Validation of Integrated Capsule-Membrane Housing

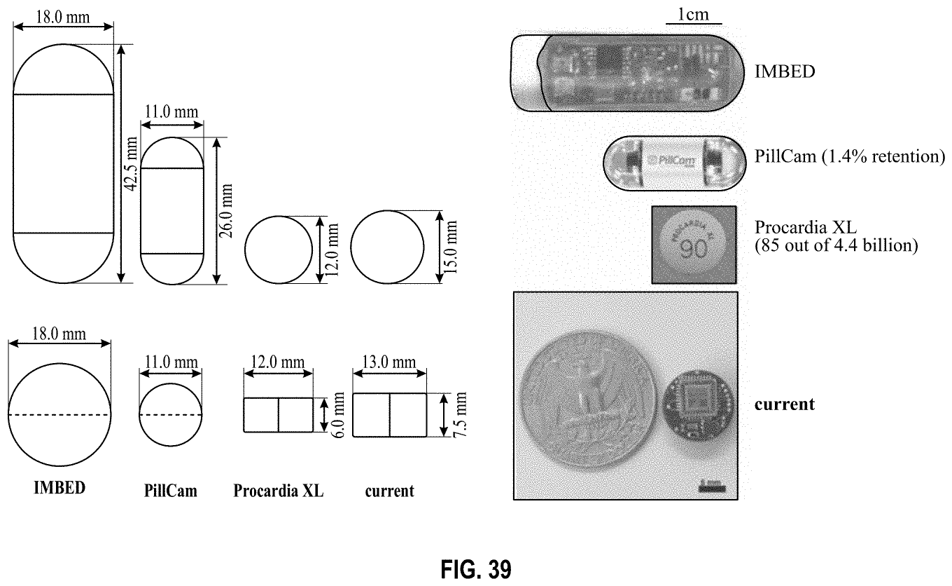

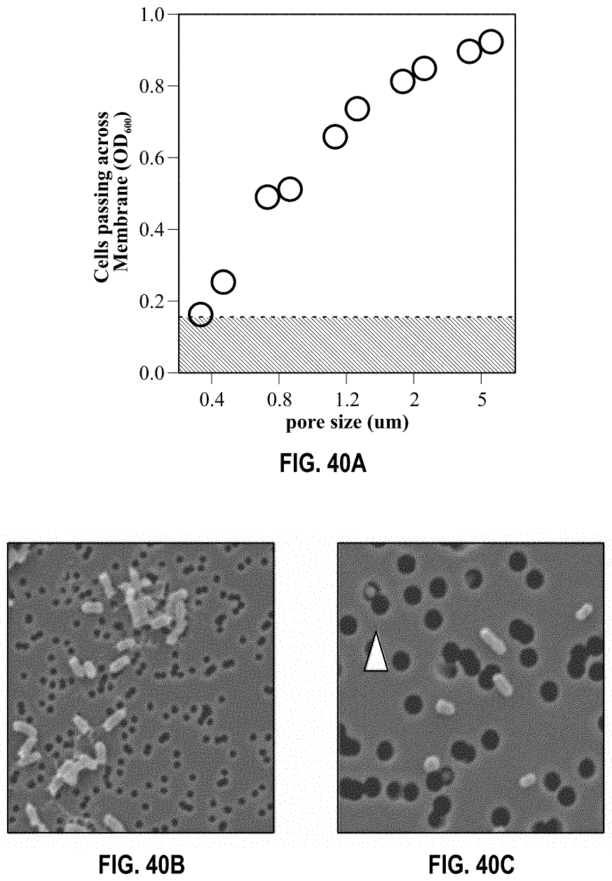

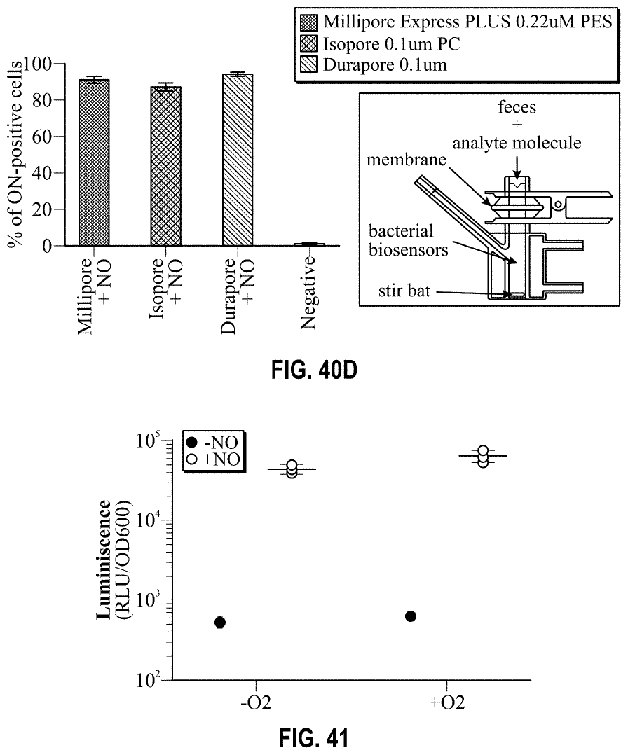

Housing on the diagnostic device should also consider the efficient exchange of nutrients and analytes and the retention of the engineered microbes. To meet this design requirement, a capsule-membrane was developed designed with dosage forms (FIG. 40A-40B), intended as a safe single-use device to be swallowed by a patient (FIG. 39).

A small tablet-shaped housing was developed that accurately aligned the bacteria to the CMOS-integrated photodiodes while maintaining a hermetic seal around the electronic components. This housing used integrated 3D printed chambers sealed with porous membranes (pore of 0.4 μm) to retain the bacterial cells (FIGS. 40A-40C) and can be easily miniaturized (FIG. 40B). The porous membranes did not interfere with detection of the target molecules when placed in feces (FIG. 40D). Furthermore, the tablet housing aligned bioluminescent bacteria with photodiodes to detect a maximum photocurrent of 1.2 pA. Chamber well thickness and cell concentration (OD=5) were parameters to consider for improved transduction of the light signal.

It was also sought to integrate the array of bacterial sensors with an ingestible electronic sensor and wireless transmission platform. Bacterial biosensors lie adjacent to photodiodes in individual wells in the device, with each biosensor arranged in a micro-chamber array 4×4. Each position in the array deploys an engineered microbial strain designed to detect one particular biomarker, i.e., NO, ROS, tetrathionate (or NO-DSS) and negative reference and trigger the expression of luciferase as a reporter output, detected by an ingestible electronic with wireless readout.

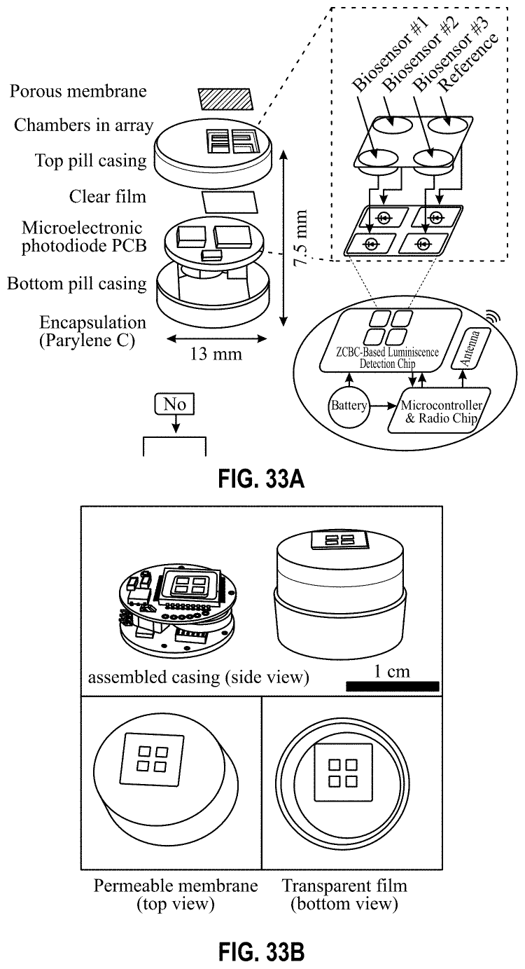

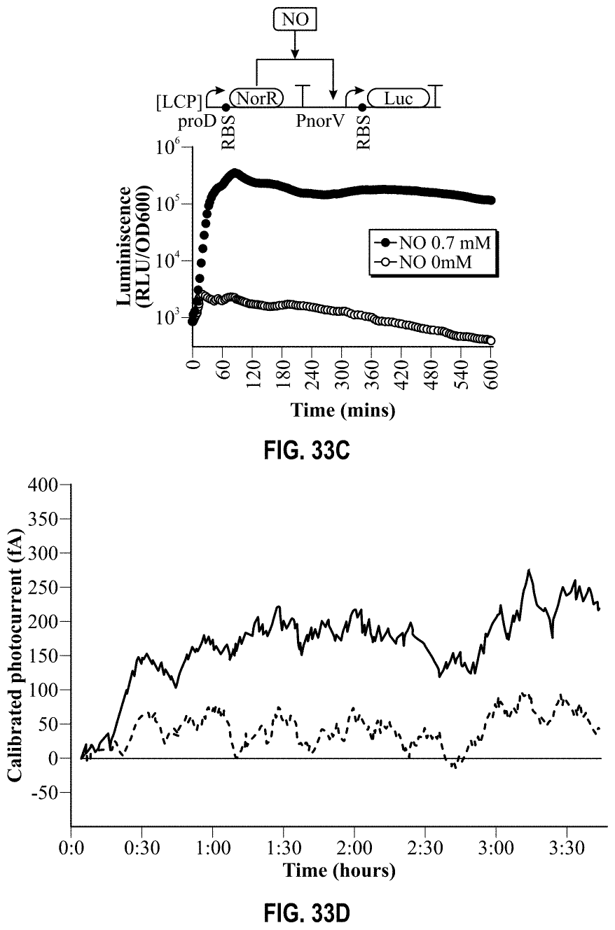

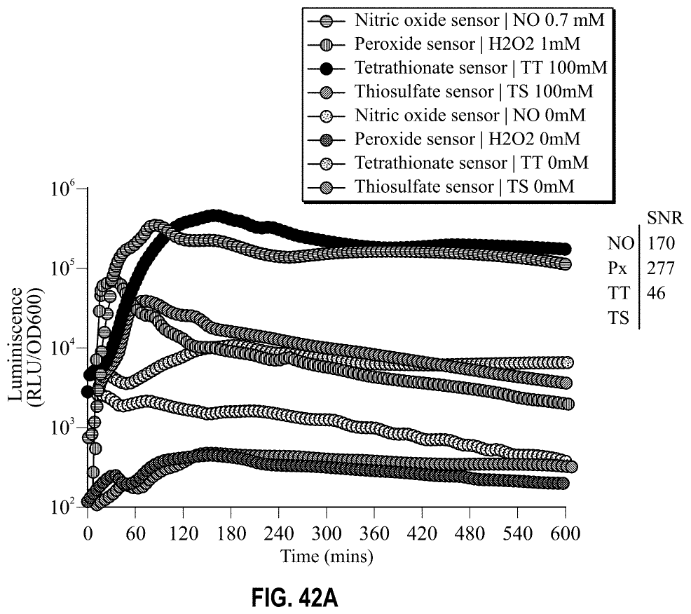

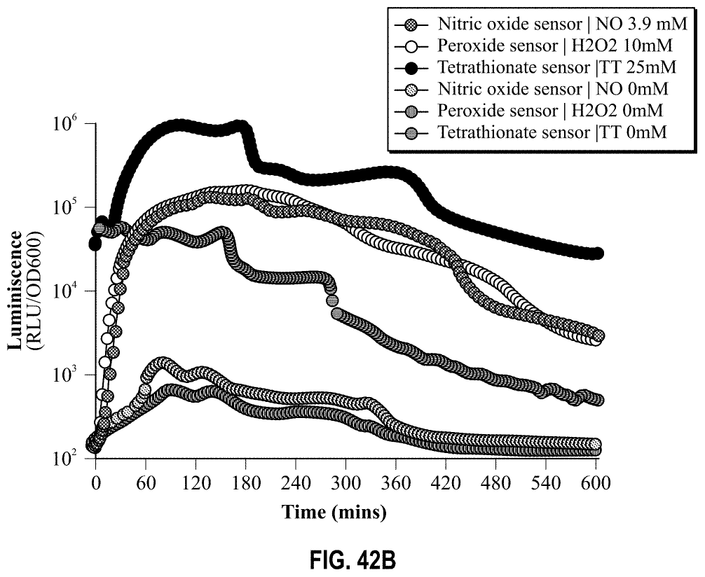

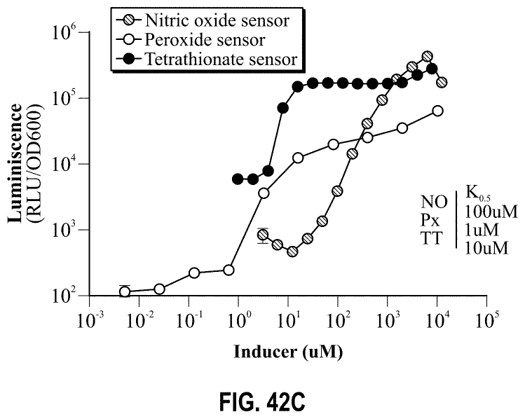

Photorhabdus luminescens luxCDABE was used as the output of the genetic circuit as it functions at body temperature and encodes the components for intracellular substrate production. This genetic circuit was constructed in the probiotic strain E. coli Nissle 1917 (Nissle V2) and exposed the resultant strain to DETA/NO as a source of NO. The NO biosensor bacteria responded rapidly to DETA/NO (tₘₐₓ=30-90 mins) with high luminescence output with a signal-to-noise ratio (SNR) of 170 (FIG. 33C). Luminescence production was also induced by DETA/NO in anaerobic conditions (FIG. 41). Similarly, the other inflammatory sensors were also built and tested in vitro (FIGS. 42A-B), being functional even in simulated intestinal fluid (FIG. 42B).

To advance the diagnostic device towards clinical application, a CMOS-integrated photodiode-based bioluminescence detector was fabricated (FIG. 33A). The chip included a switched-capacitor circuit, time-to-digital converter, voltage references, and regulators. In addition to the chip, the whole system also consists of a microcontroller that enables wireless data transmission. Bioluminescence from activated cells was detected by photodiodes located below each chamber. Custom-designed electronics processed the luminescence data by periodically sampling the photon-generated charge (continuous cycles of 10 mins with four consecutive measurements from each channel, with a programmable integration time≈26 s). The detected luminescence was converted to a digital code by the low power luminometer chip and transmitted wirelessly for calibration, display, and recording.

In this example, integrating four 1 mm by 1 mm CMOS-compatible photodiodes at each corner of the design, together with the discrete-time signal processing circuits, allowed for miniaturization of the capsule to millimeter (mm) scale, e.g., 13 mm by 7.5 mm. Advantageously, it enabled simultaneous readout of multiple analytes on a stringent power budget for a fully self-powered operation. Integrated photodiodes simplify the scalability of the biomarker validation platform since the mm-scale photodiodes could be fabricated in high throughput with CMOS technology rather than manually soldered onto PCBs.

In electrical and optical measurement to verify design functionality and obtain performance results including minimum detectable signal, noise, and power consumption/a minimum detectable signal of 78 fA, a system with a resolution of 1.7 fA/μs, and a power consumption of 58 nW with 2.5 V power supply was designed. A net 728 fA photocurrent produced by green LED supported 2 V across it. Optimized dosage forms showed activation upon tetrathionate induction. Here, a net 250 fA photocurrent was recorded, produced by the tetrathionate bacteria sensor (FIG. 33D).

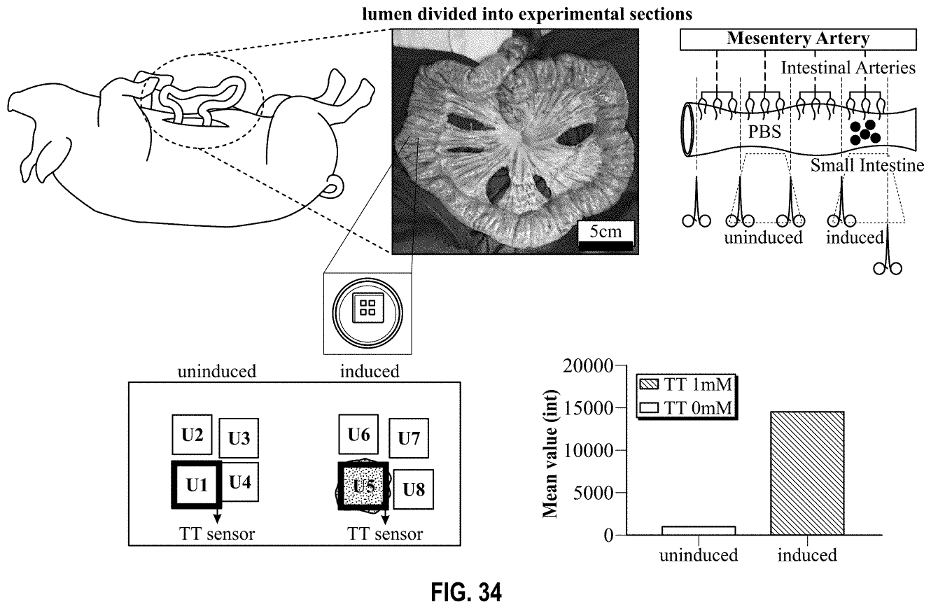

When tested in pigs, upon induction in the clamped compartments, luminescence was detected by the electronic readout circuits in the capsule; the information was wirelessly transmitted in real time from inside the body of the pig to an external recording device. This design allowed remote monitoring of biomolecules in the gut for four hours (FIG. 34).

### Discussion

An ingestible microbial biosensor was built that can sense an array of biomarkers in situ, as they are being produced inside the body. This tool can provide quantitative, real-time and multiplexed information, for example, linking gut microbiome perturbations to disease to improve disease management.

To validate the cell-based biosensors in preclinical disease models, engineered sensing bacteria were coupled to harboring a recombinase-based memory system. The memory system records information just as metabolites are being produced in the gut, activating switches within minutes of exposure. The recombinase-based switch incorporated in the biosensors detects disease stage by discretizing the magnitude of a given biomarker. Although some of the parameters trackable with this device may have no absolute “healthy range,” patterns might be revealed in the timing of acute disease episodes (flares), allowing changes in the gut biochemical milieu to be correlated with disease symptoms. Similarly, the switch acts as a peak detector for sensing and recording maximum levels of intestinal gas.

Advances in miniaturizing electronic devices enabled ingestible capsules to be used to deliver sensors to the GI tract with ultra-low power wireless circuits. CMOS-compatible photodiodes were integrated with discrete-time signal processing circuits, which made it possible to miniaturize the capsule to millimeter (mm) scale and simultaneously obtain readouts of multiple analytes on a stringent power budget (nW range) for self-powered operation. The mm-scale photodiodes can be fabricated in high throughput with CMOS technology rather than conventional commercial phototransistors with a predefined footprint, which may simplify scaling-up of the devices. Bacteria (e.g., engineered bacteria) are packaged in a small, ingestible capsule along with miniaturized electronics that transmit the detected luminescence signal wirelessly to an external device. The low power electronics work on tiny integrated solid-state batteries lasting 38 days; because batteries can be replaced with energy-harvesting technologies, the device can be powered without battery replacement for at least several weeks, potentially being used as an implant, as well.

This biosensor device thus advantageously overcomes several limitations of microbiome characterization by 16S RNA or metagenomic sequencing, as well as those of existing ingestible biosensors, which can detect microbial metabolites but require the complex analysis of bacterial gene expression or DNA in stool.

The diagnostic accuracy and specificity of these biosensors are based on the simultaneous testing of host by-products of inflammation (e.g., NO and ROS), intestinal gases (e.g., H₂S measured through thiosulfate) and microbiota-related biomolecules (e.g., tetrathionate). These biosensors can detect these labile disease mediators in the same time-frame. As biomarker levels may vary greatly among patients, a test panel of biomarkers is required to diagnose IBD and other multi-faceted diseases. These devices may be adapted to track multiple disease-associated molecules.

Since this wireless medical device for diagnostics may collect private and/or sensitive data, security was also a considered component. Given the energy and resource constraints of these medical sensors, as well as new security attack models, software-only solutions are not sufficient to establish end-to-end secure systems over a wireless link. Effective countermeasures may require leveraging the physical layer, including the hardware and the wireless communication medium, which are entirely independent from, and hence compatible with, higher-layer encryption techniques. For instance, a denial-of-service attack (e.g., a jamming signal) on the wireless communication link connecting an ingestible device to its controller may make it impossible to retrieve monitoring data.

The biosensor described herein can be developed to serve as a first line, at-home screen that would allow non-invasive continuous monitoring of the chemical environment of the GI tract. It also could offer an inexpensive point-of-care (POC) alternative to imaging capsules for endoscopy and colonoscopy. Biosensors could be designed to detect numerous biomarkers present in the GI tract, including small molecules, electrolytes, physiological gases, proteins, and DNA, which can potentially be leveraged to assess health and disease states in real time. This device can be tested in animal models for biomarker discovery for many microbiome-linked diseases. The ingestible device potentially may also offer a route for the noninvasive evaluation of the intestinal milieu. While in transit through the intestines, the capsule could determine its location (e.g., a position) in the digestive tract, detect small molecules, and perform cell-based computations to measure concentrations of intestinal biomarkers or other functions, such as AND-gates to determine co-localization of biomarkers for understanding metabolic pathways. Tracking may enable patients to make informed decisions regarding diet, lifestyle, and other interventions that require routine screening to improve health outcomes. This device could be customized for the diagnosis and management of numerous GI disorders.

### Materials and Methods

### Bacterial Strains and Culture Conditions

Routine cloning and plasmid propagation was performed in E coli E. cloni 10G (Lucigen). Gene circuits built on plasmids were transformed into probiotic E. coli Nissle 1917 for the in vitro and in vivo examples. Cells were routinely cultured at 37° C. in Luria-Bertani (LB) media (Difco). Where appropriate, growth media was supplemented with antibiotics at the following concentrations: 50 μg/mL kanamycin, 100 μg/mL carbenicillin, 25 μg/mL chloramphenicol and/or 100 μg/mL spectinomycin.

### Plasmids Construction and Circuit Characterization

Plasmids were constructed by combining PCR fragments generated by Phusion High-Fidelity DNA Polymerase (NEB) using Gibson Assembly starting from DNA sources or from gBlocks manufactured by IDT. Assembly products were transformed into chemically competent E. coli E. cloni 10G and sequences were confirmed using Sanger sequencing.

For circuit characterization of the constructs built in conjunction with the memory system, single colonies were inoculated into Teknova Hi-Def Azure Media with appropriate antibiotics and 0.2% glucose (w/v), and incubated shaking aerobically for 16-18 h at 37° C. Cultures were then diluted 2,500× into fresh Hi-Def Azure Media with appropriate antibiotics and 0.2% glucose (w/v), and shaken for 20 min aerobically at 37° C. After 20 min, 200 mL of culture was transferred to a 96-well plate, and the respective inducers, H₂O₂(Sigma-Aldrich H1009-100ML), DETA/NO (diethylenetriamine/nitric oxide adduct DETA/NO (diethylenetriamine/nitric oxide adduct Sigma-Aldrich D185-50MG), potassium tetrathionate (Sigma-Aldrich P2926-100G) and Sodium thiosulfate (Sigma-Aldrich 217263-250G) were added at appropriate concentrations via serial dilution. Plates were incubated either aerobically with shaking or anaerobically (FIG. 36B) for 20 h at 37° C. After incubation, the optical densities of cultures were measured at 600 nm in a plate reader. Cells were then assayed on the flow cytometer. For flow cytometer experiments, cells were diluted into ice-cold 1×PBS, 2% sucrose to an optical density at 600 nm of 0.02 and assayed on a BD LSRFortessa using the high-throughput sampler. At least 30,000 gated events were recorded. GFP expression was measured via the fluorescein isothiocyanate channel. FCS files were exported and processed in FlowJo software. Events were gated for live E. coli via forward scatter area and side scatter area, and then analyzed. The y axis on the flow cytometry histograms was normalized to the mode for each sample. At least three biological replicates were conducted for each experiment.

### Growth and Induction

For genetic circuit characterization, overnight cultures were diluted 1:100 in fresh LB and incubated with shaking at 37° C. for 2 hours. Cultures were removed from the incubator and 200 μL of culture were transferred to a 96-well plate containing various concentrations of inducer. The plate was returned to a shaking incubator at 37° C. Following 2 hours of incubation, luminescence was read using a BioTek Synergy H1 Hybrid Reader using a is integration time and a sensitivity of 150. Luminescence values, measured in relative luminescence units (RLUs), were normalized by the optical density of the culture measured at 600 nm.

For in vitro kinetic studies, subcultured cells were mixed with an inducer in a 96-well plate and immediately placed in the plate reader set at 37° C. without shaking. Luminescence and absorbance was read at 3 minute intervals.

### Mouse Experiments

All mouse experiments were approved by the Committee on Animal Care at the Massachusetts Institute of Technology. Male C57BL/6J mice (8-10 weeks of age) were purchased from Jackson Labs (Stock No: 000664) and were housed and handled under conventional conditions. Mice were acclimated to the animal facility 1 week prior to the commencement of experiments. Animals were randomly allocated to experimental groups. Researchers were not blinded to group assignments. Overnight cultures of E. coli Nissle grown in Teknova Hi-Def Azure Media with appropriate antibiotics and 0.2% glucose (w/v) were centrifuged at 5000 g for 5 minutes and resuspended in an equal volume of 20% sucrose. Animals were inoculated with 200 μL of bacteria culture (approximately 108 CFU) by oral gavage. Fecal pellets were collected 6 hours post-gavage and homogenized in 1 mL of PBS with a 5 mm stainless steel bead using a TissueLyser II (Qiagen) at 25 Hz for 2 minutes. Samples were centrifuged at 500 g for 30 seconds to pellet large fecal debris. Supernatant was cultured in Teknova Hi-Def Azure Media with appropriate antibiotics and 0.2% glucose (w/v) and incubated shaking aerobically for 16-18 h at 37 C. Cells were then assayed on the flow cytometer.

### Preparation of Capsules

The electronics in the capsules consisted of four photodiodes, a custom bioluminescence detector chip fabricated in a TSMC 65 nm process, a microcontroller (PIC12LF1840T39A, Microchip) and radio chip (XX), 915 MHz chip antenna (0915AT43A0026, Johanson Technology) and a commercial receiver was used (CC1200, Texas Instruments). The upper side of the top printed circuit boards (PCB) holds the fully quartz-lid packaged CMOS chip together with an additional on-board LDO (ADP-166, Analog Devices). The assembly was coated with 1 μm of Parylene C to act as a moisture barrier for the electronic components. Parylene C coating was performed using Specialty Coating System Labcoter 2 (PDS 2010) with the same protocol described in (24).

### In Vitro IMBED Experiments

LB culture media was pre-warmed for at least 2 hours prior to the start of experiments. For sensor experiments, overnight cultures were diluted 1:10 in LB, subcultured for 20 mins, concentrated 1000× by centrifugation and 1 μL of concentrated culture was added to wells in the cell carrier. Wild-type E. coli Nissle 1917 was added in the reference channel for the experiments. Once the four channels were loaded, the cell carrier was fastened to the capsule and fully submerged in pre-warmed media. LB culture media supplemented with inducer (100 mM tetrathionate, 20 mM nitric oxide, 1 mM H₂O₂ or 100 mM thiosulfate). Cultures were wrapped several times in thick black fabric to block external light, placed in an incubator at 37° C. and data was collected wirelessly for 2 hours. At the end of the experiment, devices were dissembled and cell carriers were discarded. Capsules were sterilized with 70% ethanol and thoroughly washed with distilled water. Capsules were left to air-dry and re-used for future experiments.

### Data Analysis, Statistics and Computational Methods

Data were analyzed using GraphPad Prism version 9.1.2 (Graph Software, San Diego, Calif., USA, www.graphpad.com). Sequence analysis was performed using SnapGene version 5.1.2 (www.snapgene.com). As noted, error bars represent the SEM of at least three independent experiments carried out on different days. Significance between groups was determined using an unpaired, two-tailed Student's t-test assuming unequal variance. Fold change or signal-to-noise ratio was determined by dividing the normalized luminescence values (RLU/CFU) of samples treated with the maximal inducer concentration with uninduced samples. Response curves were fit to a Hill function: Y=(BmaxXn)/(Kn+Xn)+C, where X is the inducer concentration, Y is the normalized luminescence output, Bmax is the maximum luminescence, K is the threshold constant, n is the Hill coefficient and C is the baseline luminescence.

### Additional Exemplary Embodiments

In some embodiments, a miniaturized, wireless, ingestible biosensor capsule to measure in vivo biomarkers of intestinal inflammation in a subject is described, the capsule comprising a housing, at least one biosensor component, and an electric component, wherein (a) the biosensor component comprises a biosensing genetic circuit capable of detecting biomarkers of intestinal inflammation, said circuit undergoing luminescence upon detection of said biomarkers and communicating said luminescence to the electric component, and (b) the electric component comprises an electronic readout circuit to detect luminescence and low-power electronics to process the luminescence data by periodically sampling a photon-generated charge and transmitting the information wirelessly from inside of the subject's body to an external device, wherein the capsule measures no more than 15 mm in diameter and no more than 8 mm in height.

In some embodiments, the intestinal inflammation biomarkers of some of the capsules are selected from the group consisting of intestinal gases, metabolic by-products, and microbiota-related molecules.

In some embodiments, the intestinal inflammation biomarkers of some of the capsules are selected from the group consisting of nitric oxide, H₂O₂, ROS, thiosulfate and tetrathionate.

In some embodiments, the biosensing genetic circuit of some of the capsules comprises probiotic bacteria engineered to respond to biomarkers of intestinal inflammation.

In some embodiments, the capsules further comprise memory circuits to report exposure to the biomarkers.

In some embodiments, the engineered sensor of some of the capsules does not include a fumarate and nitrate reductase regulator (FNR).

In some embodiments, the external device of some of the capsules is a smart phone.

In some embodiments, the electronic component of some of the capsules further comprises a CMOS-integrated photodiode-based bioluminescence detector, a switched-capacitor circuit, time-to-digital converter, voltage references, regulators, and/or a microcontroller that enables wireless data transmission.

In some embodiments, some of the capsules further comprise a low-power luminometer chip.

In some embodiments, a method of monitoring the health of a subject comprising orally administering some ingestible capsules to a subject and measuring the level of luminescence, wherein the level of luminescence above a threshold level indicates inflammation.

In some embodiments, the method of detecting the presence of IBD in a subject further comprises orally administering the ingestible capsule to a subject to monitor for the presence of biomarkers of intestinal inflammation and detecting IBD if biomarkers of intestinal inflammation are detected.

In some embodiments, the intestinal inflammation biomarkers of the method are selected from the group consisting of intestinal gases, metabolic by-products, and microbiota-related molecules.

In some embodiments, the intestinal inflammation biomarkers of the method are selected from the group consisting of nitric oxide, H₂O₂, ROS, thiosulfate and tetrathionate.

In some embodiments, a method of treating IBD in a subject is described comprising orally administering an ingestible capsule to monitor for the presence of biomarkers of intestinal inflammation and administering to the subject therapeutics for IBD if the biomarkers of intestinal inflammation are detected.

In some embodiments, the intestinal inflammation biomarkers of a method are selected from the group consisting of intestinal gases, metabolic by-products, and microbiota-related molecules.

In some embodiments, the intestinal inflammation biomarkers of a method are selected from the group consisting of nitric oxide, H₂O₂, ROS, thiosulfate and tetrathionate.

### Example 4

The following example describes the design and fabrication of several biosensors configured to detect one or more biomarkers.

Continuous and minimally invasive monitoring of inflammation in the gastrointestinal (GI) tract can be achieved via ingestible bioengineered sensors tailored to each disease, such as Crohn's disease. High-resolution biomolecular sensing could indicate the onset of acute disease episodes and allow for early intervention and better disease managemenFt. This example aims to detect important but labile biomarkers in the GI tract that quickly degrade in the digestive system. For example, bleeding in the upper GI tract cannot be easily detected from stool samples and requires invasive endoscopy or laboratory analysis. Alternatively, ingestible video capsule endoscopes leverage wireless cameras for image-based monitoring of the small intestine, which is traditionally difficult to access via endoscopy. However, neither of these methods can directly measure molecular mediators of disease. In contrast, the approach described in this example can detect labile biomarkers in situ via an ingestible capsule consisting of cell-based biosensors and low-power electronics.

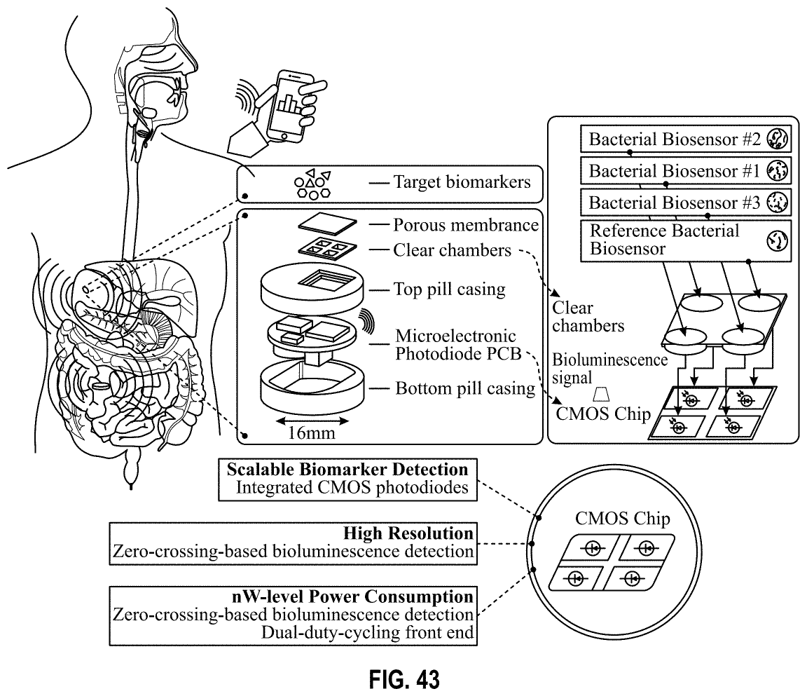

To achieve the design of these miniaturized (mm-scale) ingestible capsules as shown in FIG. 43, this example presents an ultra-low-power (nW) bioluminescence detector consisting of a CMOS-integrated p+/nwell/psub photodiode array and discrete-time photocurrent processing circuits for multiplexed measurements of disease biomarkers and continuous monitoring of disease progression. Prior works demonstrated mW-level bioluminescence and fluorescence detection using integrated photodiodes and an OTA-based readout, and nW-level detection using off-chip commercial phototransistors. The features designed herein enhance the performance relative to the prior solutions: (1) a CMOS-integrated 2 by 2 photodiode array to provide a scalable biomarker detection platform, (2) a zero-crossing-based bioluminescence detection to achieve high resolution with low energy consumption, (3) a dual-duty cycling front end to reduce average power consumption. The system is characterized by using laboratory equipment and whole-cell bacterial sensors genetically-engineered for blood detection for proof of concept of this scalable biomarker detector.

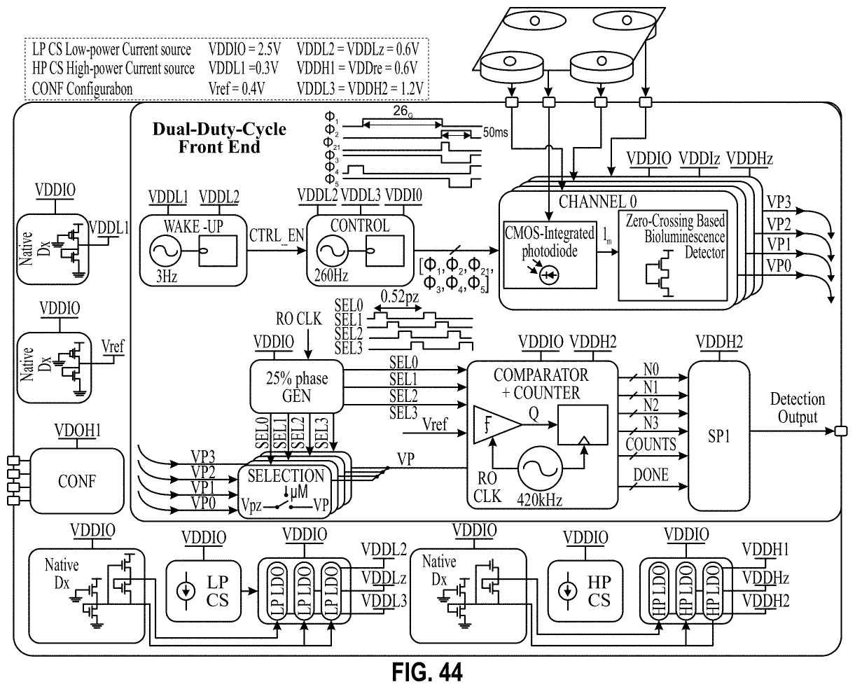

FIG. 44 show the architecture of the bioluminescence detector. Gate-leakage-based oscillators at 3 Hz and 260 Hz coupled with digital control circuits serve as wake-up control (CTRL_EN) and zero-crossing-based bioluminescence detector control signals (Φ1 to Φ5) respectively. The CMOS-integrated 2 by 2 p+/nwell/psub photodiode array converts luminescence, generated from bacterial sensors arranged in custom-designed housing with microchambers placed over the photosensitive area, into photocurrents, detected by the zero-crossing based bioluminescence chip consisting of four parallel channels. Each channel outputs a step voltage (VP0-VP3), which is fed into a time-multiplexed dynamic Strong-Arm-latch comparator driven by a ring oscillator at 420 kHz.

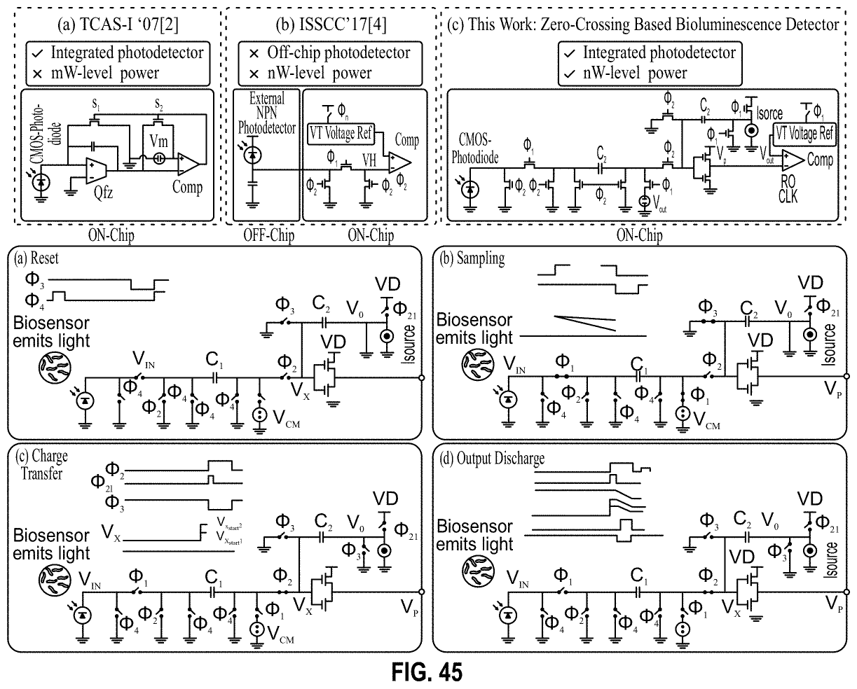

The CMOS-integrated photodiode-based detector in certain prior systems uses a continuous-time OTA-based integrator providing high gain and low noise consuming 3 mW. By contrast, this example presents a discrete-time approach using a custom zero-crossing-based detector to achieve high sensitivity (fA-level photocurrent detection) while consuming only 59 nW. FIG. 45 shows the detailed operation of the zero-crossing-based bioluminescence detector. (1 is designed to be active for a sampling period (tsampling) of 16 seconds to allow a maximum absolute 20 mV bias voltage across the photodiode for high sensitivity. The photodiode current (IPD), composed of photocurrent generated from bioluminescence (IPH) and dark current (Id), is integrated on the diode junction capacitor (CPD) over this sampling period. Negative bias voltage across the photodiode, Vin=IPD·tsampling/CPD, is sampled on the capacitor C1 when Φ1 is active, building up a total charge of Qsample=C1·(VCM+ΔVin). During charge-transfer phase, Φ2I is active for 3 ms concurrently with Φ2 (12 ms) to reset the VO node to 0.6V. The Vx node is set to Vx=[(VCM+ΔVin)·C1+VDD·C2]/(C1+C2), which is linearly proportional to Vin during the Φ2I active period. Vx discharges by a programmable current source at the rate of Isource/C1 once Φ2I is inactive. Given different initial voltages of Vx, the zero-crossing times of the inverters' threshold (Vtrip) will linearly-vary with IPD, and, hence, Vin.

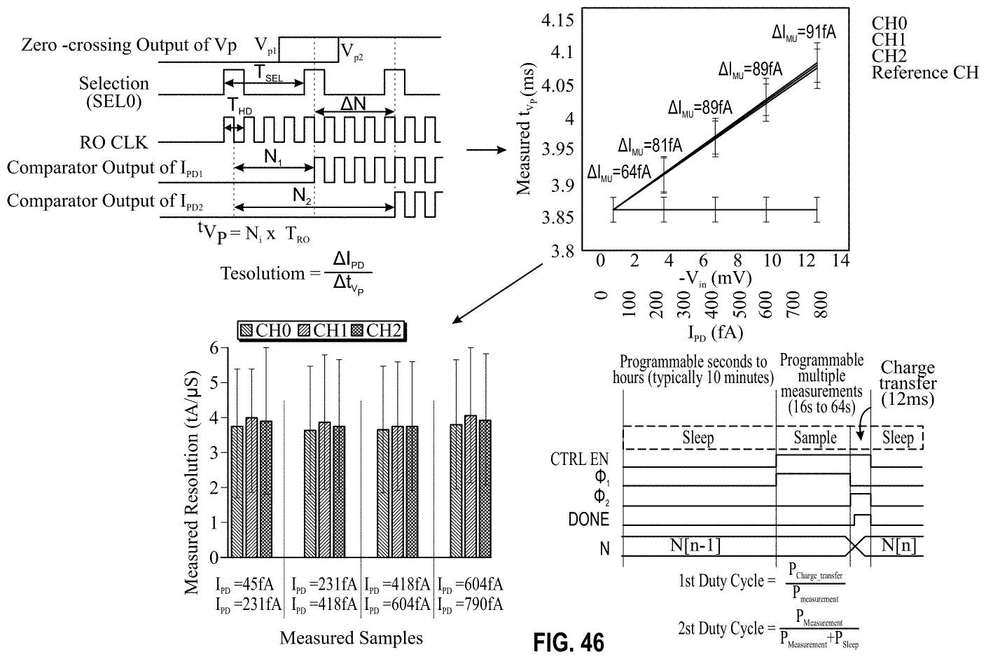

FIG. 46 shows the measured results using laboratory equipment at room temperature. A post-measurement calibration was applied, relative to the reference channel (CH3) measured counts, to eliminate the dark current dependence of the measured counts within each channel. The measured minimum detectable signal is 91 fA over 100 measurements. The measured average resolution over four channels, defined as the ratio of photocurrent variation and zero-crossing time variation, is 3.4 fA/μs. The mean value of the measured resolution per channel ranges from 3.3-3.5 fA/μs with a standard deviation range (1σ) of 1.7-1.8 fA/μs. To enable ultra-low-power readout, the CONTROL block and zero-crossing-based detectors are power gated and activated during the 16-second measurement. Further, the dual-duty cycling technique activates the 25% phase generator, comparator, and SPI blocks only during the 12 ms charge-transfer period.

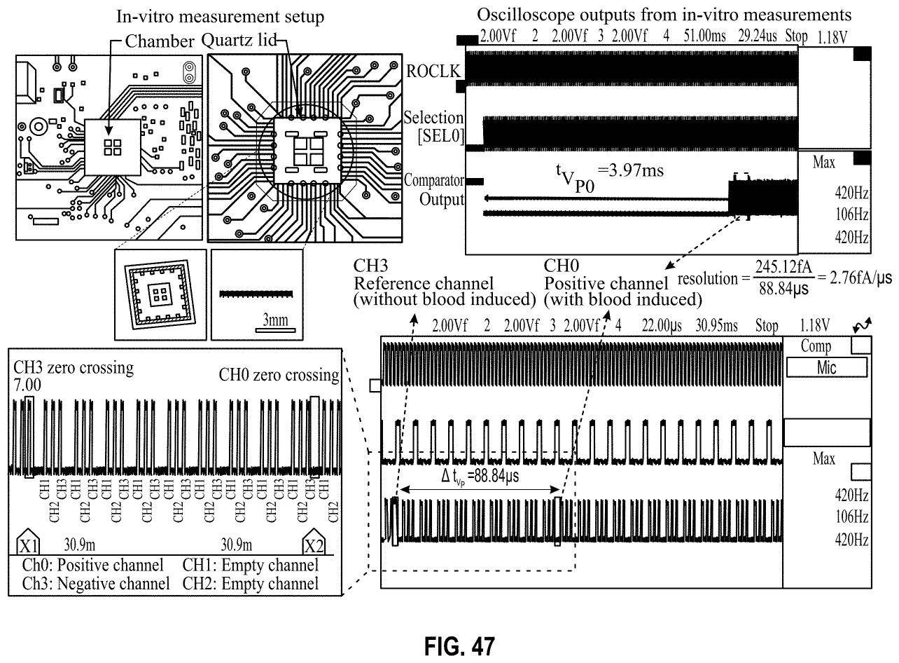

FIG. 47 demonstrates the bio-engineered sensor in-vitro testing with a laboratory strain of the heme biosensor. The heme biosensor is based on a synthetic promoter (PL(HrtO)), regulated by the Lactococcus lactis heme-responsive transcriptional repressor, HrtR, and ChuA, an outer-membrane transporter from Escherichia coli O157:H7 that allows the transit of extracellular heme through the cell envelope. The Photorhabdus luminescens luxCDABE operon is used as the output to generate bioluminescence upon activation of the synthetic promoter in the presence of induced blood. The chip is hermetically sealed under a quartz lid and is enclosed by bacterial chambers allowing the maximum light transmission to the CMOS-integrated photodiode array. The reference channel, CH3, contains an uninduced heme sensor versus CH0 contains a positively induced heme sensor (1000 ppm). Oscilloscope outputs shown in FIG. 47 demonstrated the zero-crossing time difference of these channels and the successful bioluminescence detection in CH0. The bio-engineered sensor shows a 11.8-fold measured luminescence signal compared to the measured basal expression level Neg (σ=4.2).

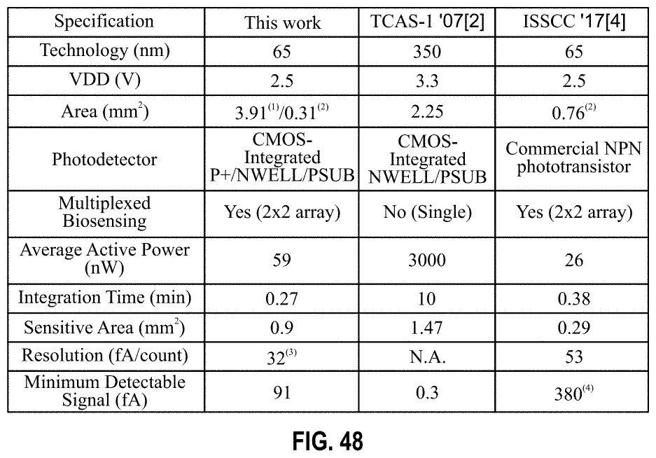

FIG. 48 compares the system performance against prior bioluminescence readout chips. This example implemented in 65 nm demonstrates CMOS-integrated photodetectors with 4.2× lower minimum detectable signal and 1.7× higher resolution compared to certain prior existing systems. Further, this chip consumes 51× lower average active power while providing a multiplexed measurement capability compared to certain prior existing systems. For an eventual wireless system, it is predicted the TX power will be approximately 145 nW for 150 bits per 16 seconds based using a crystal-less MICS TX consuming 15.5 nJ/bit at 200 kb/s. A dual-duty-cycling front end with CMOS integrated has been demonstrated photodiode array achieving high sensitivity with nW-level power consumption for minimally-invasive multiplexed biomarker detection in the GI tract for disease diagnosis and monitoring.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A system configured for gastrointestinal monitoring and/or detection of inflammatory biomarkers, the system comprising:
a capsule;
a tiered arrangement of components disposed within the capsule;
the tiered arrangement of components, comprising:
a porous membrane;
a plurality of sealable chambers;
a plurality of photodetectors, each photodetector associated with a different sealable chamber; and
a power source,
wherein at least one of the chambers of the plurality of sealable chambers is sized and adapted to contain a bacterial biosensor, and
wherein a cross-sectional dimension of the capsule is less than or equal to 15 mm.

2. A system configured for gastrointestinal sample monitoring and/or detection of inflammatory biomarkers from a sample, the system comprising:
a capsule;
a porous membrane configured to receive at least one component of the sample such that the component of the sample passes through the porous membrane;
a plurality of sealable chambers;
a plurality of photodetectors, each photodetector associated with a different sealable chamber; and
a power source,
wherein the plurality of photodetectors are integrated onto a single microelectronic chip, and
wherein at least one of the chambers of the plurality of sealable chambers is sized and adapted to contain a biosensor,
wherein the system is configured to provide greater than or equal to 0.1 fA of photocurrent detection using less than or equal to 5 mW of power.

3. A system configured for gastrointestinal monitoring and/or detection of inflammatory biomarkers in a subject, the system comprising:
a capsule sized and adapted for oral administration to the subject;
a porous membrane disposed within the capsule;
a plurality of sealable chambers,
a plurality of photodetectors, each photodetector associated with a different sealable chamber;
a power source associated with the plurality of photodetectors; and
a plurality of bacterial and/or enzymatic biosensors configured for non-blood-based detection of a gastrointestinal inflammatory process and/or disease state of the subject,
wherein each photodetector is associated with a different bacterial and/or enzymatic biosensor, such that a positive signal detected by two or more of the biosensors is correlated with the gastrointestinal inflammatory process and/or the disease state of a subject.

4. The system of claim 1, wherein at least two of the tiered arrangement of components are in contact with two other components of the tiered arrangement of components.

5. The system of claim 1, wherein an aspect ratio of at least one component of the tiered arrangement of components is greater than or equal to 1:1 and less than or equal to 10,000:1.

6. The system of claim 1, wherein an axis line passes through the porous membrane, at least one sealable chamber, at least one photodetector, and the power source.

7. The system of claim 1, wherein a cross-sectional dimension of the capsule is less than or equal to 15 mm and/or greater than or equal to 1 mm.

8. The system of claim 1, wherein two or more components of the tiered arrangement of components are oriented along an axis.

9. The system of claim 1, wherein at least one of the photodetectors of the plurality of photodetectors comprises a photodiode.

10. The system of claim 1, wherein at least one of the photodetectors comprises CMOS-integrated photodiode.

11. The system of claim 1, wherein the CMOS-integrated photodiode is electrically coupled to a controller.

12. The system of claim 1, wherein pores of the porous membrane have an average cross-sectional dimension of greater than or equal to 0.1 μm and/or less than or equal to 1.0 μm.

13. The system of claim 1, wherein the bacterial biosensor comprises E. coli.

14. The system of claim 1, wherein the bacterial biosensor comprises E. coli. And wherein the E. coli has been genetically modified relative to a naturally occurring variant of the E. coli.

15. The system of claim 1, wherein the biosensor comprises yeast.

16. The system of claim 1, wherein the biosensor comprises yeast and wherein the yeast has been genetically modified relative to a naturally occurring variant of yeast.

17. The system of claim 1, wherein at least one sealable chamber of the plurality of sealable chambers comprises at least two bacterial biosensors.

18. The system of claim 11, wherein controller is configured to receive data from two distinct photodetectors of the plurality of photodetectors.

19. The system of claim 1, wherein the system is configured to detect NO, a ROS, and/or hydrogen sulfide.

20. The system of claim 1, wherein at least one of the sealable chambers of the plurality of sealable chambers has a volume of greater than or equal to 0.1 μL and less than or equal to 5 μL.


SEQUENCE LISTING (20 sequences)

SEQ ID NO: 1 - DNA, 100 bp, Artificial Sequence
  Synthetic
ttcattatccatcctccatcgccacgatagttcatggcgataggtagaatagcaatgaacgattatccctatcaagcattctgactgagcattgctcaca

SEQ ID NO: 2 - DNA, 918 bp, Artificial Sequence
  Synthetic
atgaatattcgtgatcttgagtacctggtggcattggctgaacaccgccattttcggcgtgcggcagattcctgccacgttagccagccgacgcttagcgggcaaattcgtaagctggaagatgagctgggcgtgatgttgctggagcggaccagccgtaaagtgttgttcacccaggcgggaatgctgctggtggatcaggcgcgtaccgtgctgcgtgaggtgaaagtccttaaagagatggcaagccagcagggcgagacgatgtccggaccgctgcacattggtttgattcccacagttggaccgtacctgctaccgcatattatccctatgctgcaccagacctttccaaagctggaaatgtatctgcatgaagcacagacccaccagttactggcgcaactggacagcggcaaactcgattgcgtgatcctcgcgctggtgaaagagagcgaagcattcattgaagtgccgttgtttgatgagccaatgttgctggctatctatgaagatcacccgtgggcgaaccgcgaatgcgtaccgatggccgatctggcaggggaaaaactgctgatgctggaagatggtcactgtttgcgcgatcaggcaatgggtttctgttttgaagccggggcggatgaagatacacacttccgcgcgaccagcctggaaactctgcgcaacatggtggcggcaggtagcgggatcactttactgccagcgctggctgtgccgccggagcgcaaacgcgatggggttgtttatctgccgtgcattaagccggaaccacgccgcactattggcctggtttatcgtcctggctcaccgctgcgcagccgctatgagcagctggcagaggccatccgcgcaagaatggatggccatttcgataaagttttaaaacaggcggtttaa

SEQ ID NO: 3 - DNA, 717 bp, Artificial Sequence
  Synthetic
atgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttcggttatggtgttcaatgctttgcgagatacccagatcatatgaaacagcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgatctctacaaataa

SEQ ID NO: 4 - DNA, 1542 bp, Artificial Sequence
  Synthetic
atgagagccctggtagtcatccgcctgtcccgcgtcaccgatgctacgacttcaccggagcgtcagctggagtcttgccagcagctctgcgcccagcgcggctgggacgtcgtcggggtagcggaggatctggacgtctccggggcggtcgatccgttcgaccggaagcgcagaccgaacctggcccggtggctagcgttcgaggagcaaccgttcgacgtgatcgtggcgtaccgggtagaccggttgacccgatcgatccggcatctgcagcagctggtccactgggccgaggaccacaagaagctggtcgtctccgcgaccgaagcgcacttcgatacgacgacgccgtttgcggcggtcgtcatcgcgcttatgggaacggtggcgcagatggaattagaagcgatcaaagagcggaaccgttcggctgcgcatttcaatatccgcgccgggaaataccgaggatccctgccgccgtggggatacctgcctacgcgcgtggacggggagtggcggctggtgccggaccctgtgcagcgagagcgcatcctcgaggtgtatcaccgcgtcgtcgacaaccacgagccgctgcacctggtggcccacgacctgaaccggcgtggtgtcctgtcgccgaaggactacttcgcgcagctgcaaggccgcgagccgcagggccgggagtggtcggctaccgcgctgaagcgatcgatgatctccgaggcgatgctcgggtacgcgactctgaacggtaagaccgtccgagacgacgacggagccccgctggtgcgggctgagccgatcctgacccgtgagcagctggaggcgctgcgcgccgagctcgtgaagacctcccgggcgaagcccgcggtgtctaccccgtcgctgctgctgcgggtgttgttctgtgcggtgtgcggggagcccgcgtacaagttcgccgggggaggacgtaagcacccgcgctaccgctgccgctcgatggggttcccgaagcactgcgggaacggcacggtggcgatggccgagtgggacgcgttctgcgaggagcaggtgctggatctgctcggggacgcggagcgtctggagaaagtctgggtagccggctcggactccgcggtcgaactcgcggaggtgaacgcggagctggtggacctgacgtcgctgatcggctccccggcctaccgggccggctctccgcagcgagaagcactggatgcccgtattgcggcgctggccgcgcggcaagaggagctggagggtctagaggctcgcccgtctggctgggagtggcgcgagaccgggcagcggttcggggactggtggcgggagcaggacaccgcggcaaagaacacctggcttcggtcgatgaacgttcggctgacgttcgacgtccgcggcgggctgactcgcacgatcgacttcggggatctgcaggagtacgagcagcatctcaggctcggcagcgtggtcgaacggctacacaccgggatgtcgaggcctgcagcaaacgacgaaaactacgctgcagcagtttag

SEQ ID NO: 5 - DNA, 68 bp, Artificial Sequence
  Synthetic
ttgacagctagctcagtcctaggtattgtgctagcctagtatcgatctccataactatcctatagatc

SEQ ID NO: 6 - DNA, 69 bp, Artificial Sequence
  Synthetic
tttacggctagctcagtcctaggtactatgctagcagaaatataaagaacgatctatttatccgcgtac

SEQ ID NO: 7 - DNA, 1785 bp, Artificial Sequence
  Synthetic
atgtcccgcctgctgctgtgtatctgtgttctgctgttctcttctgtggcgtggtctaaaccgcagcagttttatgtgggcgtactggctaactggggtcatcagcaagccgttgaacgttggaccccgatgatggagtatctgaacgaacatgtgccggacgcggaatttcacgtctacccgggcaacttcaaagcactgaacctggcaatggaactgggccagattcagttcattatcactaacccgggccaatatctgtacctgagcaatcagtacccgctgtcttggctggcgaccatgcgttctaagcgtcacgatggtaccacttctgcgatcggttccgccattattgtccgcgcggacagcgactaccgcaccctgtacgacctgaaaggtaaagtggtggctgcgtccgacccgcatgctctgggtggctaccaagcgaccgtcggtctgatgcattccctgggcatggatccggacaccttcttcggtgaaaccaagtttctgggctttccactggatccgctgctgtaccaagttcgtgatggcaacgttgacgcggccattaccccactgtgcactctggaggacatggttgcacgcggcgtactgaaatcttccgattttcgtgtgctgaaccctagccgcccggatggtgtagaatgccagtgctctaccaccctgtacccgaactggtctttcgctgcgactgagtctgtatccaccgaactgtctaaagaaatcacgcaggcactgctggaactgccatccgacagcccggcagctatcaaagcgcaactgaccggctggaccagcccgatctcccaactggcggtaatcaaactgttcaaagagctgcacgtaaaaaccccggactctagccgttgggaagccgttaagaagtggctggaagaaaaccgtcactggggtatcctgtctgttctggtgttcatcattgcaacgctgtatcacctgtggattgaataccgcttccaccaaaaaagctcttctctgatcgaatctgaacgtcagctgaaacagcaagctgttgccctggaacgtctgcaatctgctagcatcgttggtgaaattggtgcgggtctggcccacgagattaatcagccgatcgctgcaattacctcttattctgaaggtggcatcatgcgcctgcaaggtaaagaacaggcggatacggatagctgcatcgaactgctggaaaaaatccacaaacagagcactcgcgcaggcgaagtggtgcaccgcatccgtggtctgctgaaacgtcgtgaagcggtgatggtagatgttaacatcctgaccctggtggaagaatccatcagcctgctgcgtctggagctggcacgtcgcgaaatccagatcaacactcagatcaaaggtgaaccgttcttcattactgccgaccgcgttggcctgctgcaagttctgattaacctgatcaaaaactccctggacgcgatcgctgaatctgataatgcccgttctggtaaaatcaacatcgaactggactttaaagagtaccaggtaaacgtctccatcatcgataacggtccgggcctggcgatggattctgacactctgatggctacgttttacactaccaaaatggatggcctgggcctgggtctggcaatctgccgcgaagttatcagcaaccacgacggccacttcctgctgtccaaccgtgacgacggcgttctgggctgtgtggcaaccctgaatctgaaaaaacgcggttctgaagtgccgatcgaagtctaa

SEQ ID NO: 8 - DNA, 612 bp, Artificial Sequence
  Synthetic
atgcagcagcaaatcaacggcccggtctacctggtggatgatgatgaagccattatcgactccatcgattttttgatggagggctacggttacaaactgaactcgtttaactgcggcgatcgctttttggcagaagtcgatctcacccaggcaggatgtgtaattctggatgcgcgtatgccaggcttaactggtcctcaggtgcaacagctgctgagcgacgcgaaaagcccgcttgcggtcatcttcctgaccggccatggcgatgttccgatggcggttgatgcgttcaaaaatggcgcgttcgatttctttcaaaaacctgtgccgggtagcttgctcagtcagtcaattgccaaaggcttgacttattcaatcgatcaacatctgaaacgtactaaccaagcgttaatcgacacgctctcggaacgcgaagctcaaatttttcaactggtgattgcaggcaacaccaacaaacagatggctaacgagctttgcgtggctattcgtaccattgaggttcaccgtagcaaactgatgaccaaactgggtgttaacaacctggctgaactggttaaactggcgccgctgctggcacataaatccgaataa

SEQ ID NO: 9 - DNA, 342 bp, Artificial Sequence
  Synthetic
ttcaagcattattatgctgttttttgaagtgaatgtgcggccatctagccgcacattttgcatctaaaacatgcagtcatcagcaaaataataaacttttccccaatatgtggtttaccacaatttacaggaattcactcctgtggtggtgcaaatttgaactgtgaattgcttcacaaacgccgctatcgcaatgtcagtatgtggtttaccacaatatctaatatcactctgctcaataacaatgatgaaaaccttaggaagaagttaattgtgttaaacagttaactaggggctttatctaacgctctcctaaggacaactgtcattgggagatttaac

SEQ ID NO: 10 - DNA, 5798 bp, Artificial Sequence
  Synthetic
atgactaaaaaaatttcattcattattaacggccaggttgaaatctttcccgaaagtgatgatttagtgcaatccattaattttggtgataatagtgtttacctgccaatattgaatgactctcatgtaaaaaacattattgattgtaatggaaataacgaattacggttgcataacattgtcaattttctctatacggtagggcaaagatggaaaaatgaagaatactcaagacgcaggacatacattcgtgacttaaaaaaatatatgggatattcagaagaaatggctaagctagaggccaattggatatctatgattttatgttctaaaggcggcctttatgatgttgtagaaaatgaacttggttctcgccatatcatggatgaatggctacctcaggatgaaagttatgttcgggcttttccgaaaggtaaatctgtacatctgttggcaggtaatgttccattatctgggatcatgtctatattacgcgcaattttaactaagaatcagtgtattataaaaacatcgtcaaccgatccttttaccgctaatgcattagcgttaagttttattgatgtagaccctaatcatccgataacgcgctctttatctgttatatattggccccaccaaggtgatacatcactcgcaaaagaaattatgcgacatgcggatgttattgtcgcttggggagggccagatgcgattaattgggcggtagagcatgcgccatcttatgctgatgtgattaaatttggttctaaaaagagtctttgcattatcgataatcctgttgatttgacgtccgcagcgacaggtgcggctcatgatgtttgtttttacgatcagcgagcttgtttttctgcccaaaacatatattacatgggaaatcattatgaggaatttaagttagcgttgatagaaaaacttaatctatatgcgcatatattaccgaatgccaaaaaagattttgatgaaaaggcggcctattctttagttcaaaaagaaagcttgtttgctggattaaaagtagaggtggatattcatcaacgttggatgattattgagtcaaatgcaggtgtggaatttaatcaaccacttggcagatgtgtgtaccttcatcacgtcgataatattgagcaaatattgccttatgttcaaaaaaataagacgcaaaccatatctatttttccttgggagtcatcatttaaatatcgagatgcgttagcattaaaaggtgcggaaaggattgtagaagcaggaatgaataacatatttcgagttggtggatctcatgacggaatgcgaccgttgcaacgattagtgacatatatttctcatgaaaggccatctaactatacggctaaggatgttgcggttgaaatagaacagactcgattcctggaagaagataagttccttgtatttgtcccataataggtaaaagtatggaaaatgaatcaaaatataaaaccatcgaccacgttatttgtgttgaaggaaataaaaaaattcatgtttgggaaacgctgccagaagaaaacagcccaaagagaaagaatgccattattattgcgtctggttttgcccgcaggatggatcattttgctggtctggcggaatatttatcgcggaatggatttcatgtgatccgctatgattcgcttcaccacgttggattgagttcagggacaattgatgaatttacaatgtctataggaaagcagagcttgttagcagtggttgattggttaactacacgaaaaataaataacttcggtatgttggcttcaagcttatctgcgcggatagcttatgcaagcctatctgaaatcaatgcttcgtttttaatcaccgcagtcggtgttgttaacttaagatattctcttgaaagagctttagggtttgattatctcagtctacccattaatgaattgccggataatctagattttgaaggccataaattgggtgctgaagtctttgcgagagattgtcttgattttggttgggaagatttagcttctacaattaataacatgatgtatcttgatataccgtttattgcttttactgcaaataacgataattgggtcaagcaagatgaagttatcacattgttatcaaatattcgtagtaatcgatgcaagatatattctttgttaggaagttcgcatgacttgagtgaaaatttagtggtcctgcgcaatttttatcaatcggttacgaaagccgctatcgcgatggataatgatcatctggatattgatgttgatattactgaaccgtcatttgaacatttaactattgcgacagtcaatgaacgccgaatgagaattgagattgaaaatcaagcaatttctctgtcttaaaatctattgagatattctatcactcaaatagcaatataaggactctctatgaaatttggaaactttttgcttacataccaacctccccaattttctcaaacagaggtaatgaaacgtttggttaaattaggtcgcatctctgaggagtgtggttttgataccgtatggttactggagcatcatttcacggagtttggtttgcttggtaacccttatgtcgctgctgcatatttacttggcgcgactaaaaaattgaatgtaggaactgccgctattgttcttcccacagcccatccagtacgccaacttgaagatgtgaatttattggatcaaatgtcaaaaggacgatttcggtttggtatttgccgagggctttacaacaaggactttcgcgtattcggcacagatatgaataacagtcgcgccttagcggaatgctggtacgggctgataaagaatggcatgacagagggatatatggaagctgataatgaacatatcaagttccataaggtaaaagtaaaccccgcggcgtatagcagaggtggcgcaccggtttatgtggtggctgaatcagcttcgacgactgagtgggctgctcaatttggcctaccgatgatattaagttggattataaatactaacgaaaagaaagcacaacttgagctttataatgaagtggctcaagaatatgggcacgatattcataatatcgaccattgcttatcatatataacatctgtagatcatgactcaattaaagcgaaagagatttgccggaaatttctggggcattggtatgattcttatgtgaatgctacgactatttttgatgattcagaccaaacaagaggttatgatttcaataaagggcagtggcgtgactttgtattaaaaggacataaagatactaatcgccgtattgattacagttacgaaatcaatcccgtgggaacgccgcaggaatgtattgacataattcaaaaagacattgatgctacaggaatatcaaatatttgttgtggatttgaagctaatggaacagtagacgaaattattgcttccatgaagctcttccagtctgatgtcatgccatttcttaaagaaaaacaacgttcgctattatattagctaaggagaaagaaatgaaatttggattgttcttccttaacttcatcaattcaacaactgttcaagaacaaagtatagttcgcatgcaggaaataacggagtatgttgataagttgaattttgaacagattttagtgtatgaaaatcatttttcagataatggtgttgtcggcgctcctctgactgtttctggttttctgctcggtttaacagagaaaattaaaattggttcattaaatcacatcattacaactcatcatcctgtcgccatagcggaggaagcttgcttattggatcagttaagtgaagggagatttattttagggtttagtgattgcgaaaaaaaagatgaaatgcatttttttaatcgcccggttgaatatcaacagcaactatttgaagagtgttatgaaatcattaacgatgctttaacaacaggctattgtaatccagataacgatttttatagcttccctaaaatatctgtaaatccccatgcttatacgccaggcggacctcggaaatatgtaacagcaaccagtcatcatattgttgagtgggcggccaaaaaaggtattcctctcatctttaagtgggatgattctaatgatgttagatatgaatatgctgaaagatataaagccgttgcggataaatatgacgttgacctatcagagatagaccatcagttaatgatattagttaactataacgaagatagtaataaagctaaacaagagacgcgtgcatttattagtgattatgttcttgaaatgcaccctaatgaaaatttcgaaaataaacttgaagaaataattgcagaaaacgctgtcggaaattatacggagtgtataactgcggctaagttggcaattgaaaagtgtggtgcgaaaagtgtattgctgtcctttgaaccaatgaatgatttgatgagccaaaaaaatgtaatcaatattgttgatgataatattaagaagtaccacatggaatatacctaatagatttcgagttgcagcgaggcggcaagtgaacgaatccccaggagcatagataactatgtgactggggtgagtgaaagcagccaacaaagcagcagcttgaaagatgaagggtataaaagagtatgacagcagtgctgccatactttctaatattatcttgaggagtaaaacaggtatgacttcatatgttgataaacaagaaattacagcaagctcagaaattgatgatttgattttttcgagcgatccattagtgtggtcttacgacgagcaggaaaaaatcagaaagaaacttgtgcttgatgcatttcgtaatcattataaacattgtcgagaatatcgtcactactgtcaggcacacaaagtagatgacaatattacggaaattgatgacatacctgtattcccaacatcggtttttaagtttactcgcttattaacttctcaggaaaacgagattgaaagttggtttaccagtagcggcacgaatggtttaaaaagtcaggtggcgcgtgacagattaagtattgagagactcttaggctctgtgagttatggcatgaaatatgttggtagttggtttgatcatcaaatagaattagtcaatttgggaccagatagatttaatgctcataatatttggtttaaatatgttatgagtttggtggaattgttatatcctacgacatttaccgtaacagaagaacgaatagattttgttaaaacattgaatagtcttgaacgaataaaaaatcaagggaaagatctttgtcttattggttcgccatactttatttatttactctgccattatatgaaagataaaaaaatctcattttctggagataaaagcctttatatcataaccggaggcggctggaaaagttacgaaaaagaatctctgaaacgtgatgatttcaatcatcttttatttgatactttcaatctcagtgatattagtcagatccgagatatatttaatcaagttgaactcaacacttgtttctttgaggatgaaatgcagcgtaaacatgttccgccgtgggtatatgcgcgagcgcttgatcctgaaacgttgaaacctgtacctgatggaacgccggggttgatgagttatatggatgcgtcagcaaccagttatccagcatttattgttaccgatgatgtcgggataattagcagagaatatggtaagtatcccggcgtgctcgttgaaattttacgtcgcgtcaatacgaggacgcagaaagggtgtgctttaagcttaaccgaagcgtttgatagttga

SEQ ID NO: 11 - DNA, 15 bp, Artificial Sequence
  Synthetic
attaaagaggagaaa

SEQ ID NO: 12 - DNA, 14 bp, Artificial Sequence
  Synthetic
attaaagaggagaa

SEQ ID NO: 13 - DNA, 12 bp, Artificial Sequence
  Synthetic
aaagaggggaaa

SEQ ID NO: 14 - DNA, 49 bp, Artificial Sequence
  Synthetic
cggccggcttgtcgacgacggcggtctccgtcgtcaggatcatccgggc

SEQ ID NO: 15 - DNA, 58 bp, Artificial Sequence
  Synthetic
gtcgtggtttgtctggtcaaccaccgcggtctcagtggtgtacggtacaaaccccgac

SEQ ID NO: 16 - DNA, 180 bp, Artificial Sequence
  Synthetic
acggaaaaactcatctttgcctcactgtcaatttgactatagatattgtcatatcgaccatttgattgatagtcattttgactactcattaatgggcataattttatttatagagtaaaaacaatcagataaaaaactggcacgcaatctgcaattagcaagacatctttttagaacacg

SEQ ID NO: 17 - DNA, 1566 bp, Artificial Sequence
  Synthetic
atgagtttttccgttgatgtgctggcgaatatcgccatcgaattgcagcgtgggattggtcaccaggatcgttttcagcgcctgatcaccacgctacgtcaggtgctggagtgcgatgcgtctgcgttgctacgttacgattcgcggcagtttattccgcttgccatcgacggtctggcaaaggatgtactcggtagacgctttgcgctggaagggcatccacggctggaagcgattgcccgcgccggggatgtggtgcgctttcccgcagacagcgaattgcccgatccctatgacggtttgattcctgggcaggagagtctgaaggttcacgcctgcgttggtctgccattgtttgccgggcaaaacctgatcggcgcactgacgctcgacgggatgcagcccgatcagttcgatgttttcagcgacgaagagctacggctgattgctgcgctggcggcgggagcgttaagcaatgcgttgctgattgaacaactggaaagccagaatatgctgccaggcgatgccacgccgtttgaagcggtgaaacagacgcagatgattggcttgtcccctggcatgacgcaactgaaaaaagagattgagattgtggcggcgtccgatctcaacgtcctgatcagcggtgagactggaaccggtaaggagctggtggcgaaagcgattcatgaagcctcgccacgggcggtgaatccgctggtctatctcaactgtgctgcactgctggaaagtgtggcggaaagtgagttgttcgggcatgtgaaaggagcgtatactggcgctatcagtaatcgcagcgggaagtttgaaatggcggataacggcacgctgtttctggatgagatcggcgagttgtcgttggcatcgcaggccaagctgctgagggtgttgcagtatggcgatattcagcgcgttggcgatgaccgttgtttgcgggtcgatgtgcgcgtgctggcggcgactaaccgcgatttacgcgaagaggtgctggcagggcgattccgcgccgatttgtttcatcgcctgagcgtgtttccactttcggtgccgccgctgcgtgagcggggcgatgatgtcattctgctggcggggtatttctgcgagcagtgtcgtttgcggcaggggctctcccgcgtggtattaagtgccggagcgcgaaatttactgcaacactacagttttccgggaaacgtgcgcgaactggaacatgctattcatcgggcggtagttctggcgagagccacccgcagcggcgatgaagtgattcttgaggcgcaacattttgcgcgcgcggatgctggaaaccgacgtcgccaacctgcatcggctggcgaaacgcgcgcggatgctggaaacgcgacagaagcgttccagcgtgaaactattcgtcaggcactggcacaaaatcatcacaactgggctgcctgcgcgcggatgctggaaaccgacgtcgccaacctgcatcggctggcgaaacgcgcgcggatgctggaaaccgacgtcgccaacctgcatcggctggcgaaacgtctgggattgaaggattaa

SEQ ID NO: 18 - DNA, 621 bp, Artificial Sequence
  Synthetic
atgagcctggcgctgccggtgtacctgattgacgacgatgactccgttcgccgttccctgcgtttcatgctggaaagctacggcctgaaaatcactgacttcgattctgctgaagcgttcttcaccgcggtagacctgaccctgccgggttgcgcactggtggacgtacgtatgccgggcctgagcggcccacagctgcacctggaactggtgtctaagaacagcccgctggccgtgatctatctgaccggtcacggcgacgttccgatggcggttgaagcgctgaaactgggtgcggtagatttctttcagaaacctgcagacggcgctaaactggctgaagctgtggtcaaagccctggaacacgcgaaaacccactaccaagacaaccagtacctggaaacctatcaggctctgaccccacgtgagcgcgaaatcctgaacctgattgcgcaaggtctgaaaaaccaggaaatcgcggacaacctgtgcattgcgatgcgcaccgtagaaatccaccgtgcgaacctgatgaaaggtatgcaggtgggcagcctggcagagatgatgctgatttactctcgtatcgcggagcgtctgcgcctggaagataaccgttaa

SEQ ID NO: 19 - DNA, 2016 bp, Artificial Sequence
  Synthetic
atgttttcttccggcatgagcctgaaatggcctctgagcatccagggttccatcgaaccgaaaatgactccgcgcatcatcttccgcatggccctgcgcactaaagtgttcatcaccctgctggcttgcctgtttgcttctgccaacgtattcgcggtggaaccgcagaaccagtccgcgccttccgatgtgatggacgctgaggtggttgctccggttgcgacggaggaaccgggttaccaggtcgttgatgttggtgtgctggcgattcgcggttataaagctaccatcaaccgttggcagccactgatgggttggctggagactcagatcccgaactcctacttccgcctgcacccgctgtctctggacgagctggcaaagggtgtagaaacccagggtctggacttcgtaatcaccaatccaggccagtccgtactgctggcgcgtcagtattctctgacgtggctggcgaccctgcgctctccgctgaacaacggtgctgcgatgcaggtgggttccgcgctggtagttcgtgccgacagcccgtaccagaccctgaccgatctgaaaggccgtcgtctgggcatcgtcagcaaaaacgcattcggtggttatctgacgctggtatatgaggcacagctgaaaggcatcgatctgccgcgctttgttggtgaaatcatcccactgggcttcccgctggataacctgctgtatcagctggatgatcagaaatctggtaatgacgccgcaaaagacaatcgtctggatgccaccgttgtaccggtatgccaactggaacagatgcaggccgaaggtctgatcaacatcggccactatcgcgttctggacaaccagaccccggtaggtttccactgtcaagtaagcactcgtctgtatccgaactggtctatggctaaaactaaccgtgccagccagtccctggcaaaatctgtgacccaggctctgctggctctgccggaagatcacctggcagcgaaaagctctgactctgcaggttggaccactgcagtgtcccagctggcgatcgaccagctgctgaaagacctggacatgcatccactgcagaccccatggtggcagcgcgcttggcagtgggtaaaactgcaccagcagtgggcgtggttcattctgggtatcctggtcctgctgaacgcataccatttctggctggaataccgtttctcccgtcgtggtcgtgaactgatcaacacccagcgccagctgaacgaaaaccgtgctctgctggagcacgcacaacgtatcgccatcgctggcgaactgggtgctagcctgtcccatgaactgaaccagccgctggctgcaattggtcactattgccatggtgcggaagtgcgtctgcagcgtggtacttctcctgaagaactgcagtctgttctgaccctgatccagcaggaagttacgcgcgccgactctatcatcagccgtctgcgtaacctgctgaaaaaacgcccggttagcaaacaggctctgtatctgcacgaactggtaaacgaaacggtgccgctgctggcgtacgaattcgaacagcaccagatcaacctggcggtaaacgtcagcggtgaaccttacctgcaatctctggacgaggtcggtatgcagcagctgctgctgaacctgctgaaaaacgcgtttgacgcgtgcgttcagcgcctggaactggaatcctccggcactgaacagagcggtatgcgtaaaccgtatgttcctacgatcgatattgatctgcgctaccaggagcgtactctgctgctgactgtgacggataacggcactggtctgaccgaggagactagcctgctgatgcaggccttctattccaccaaatctgaaggcctgggcctgggtctggttatttgccgtgatatcgctgagtcccacggtggcaccttctctctggaatccgccatgggtggtggctgccaggctcaggtggccatcccgcgcaagccggaaccgaatggcgttctgtaa

SEQ ID NO: 20 - DNA, 208 bp, Artificial Sequence
  Synthetic
tttatagtaaatcactgcataattcgtgtcgctcaaggcgcactcccgttctggataatgttttttgcgccgacatcataacggttctggcaaatattctgaaatgagctggttagctagtcaatatttgttgcgctagatcaaatccacgcctgatatgtggaaaaccactatagttatgccgcctcgccttttacagaatgcctgt